(19) United States
(12) Patent Application Publication
(10) Pub. No.: US 2024/0067634 A1
(43) Pub. Date: Feb. 29, 2024

(54) INHIBITORS OF NEF DOWNMODULATION

(71) Applicant: THE REGENTS OF THE UNIVERSITY OF MICHIGAN, Ann Arbor, MI (US)

(72) Inventors: David H. Sherman, Ann Arbor, MI (US); Morgan T. McCauley, Ann Arbor, MI (US); Nolan Carney, Ann Arbor, MI (US); Andrew W. Robertson, Ann Arbor, MI (US); Mark M. Painter, Ann Arbor, MI (US); Kathleen L. Collins, Ann Arbor, MI (US); Ashootosh Tripathi, Ypsilanti, MI (US); Alanna R. Condren, Ann Arbor, MI (US)

(21) Appl. No.: 18/266,688

(22) PCT Filed: Dec. 16, 2021
(86) PCT No.: PCT/US21/63709

§ 371 Date: Jun. 12, 2023

Related U.S. Application Data
(60) Provisional application No. 63/126,124, filed on Dec. 16, 2020.

(51) Int. Cl. C07D 407/06; A61P 31/18; C07D 407/14; C07H 17/04

(52) U.S. Cl. CPC C07D 407/06; A61P 31/18; C07D 407/14; C07H 17/04

(57) ABSTRACT

This disclosure relates generally to inhibitors of MHC-I downmodulation, and methods of treating or preventing an HIV infection by administering the inhibitors to a patient in need of treatment thereof.

### STATEMENT OF GOVERNMENT SUPPORT

This invention was made with government support under AI116158, AI148383 and AI131957 awarded by the National Institutes of Health. The government has certain rights in the invention.

### BACKGROUND

### Field of the Disclosure

This disclosure relates generally to inhibitors of MHC-I downmodulation, and methods of treating or preventing an HIV infection by administering the inhibitors to a patient in need of treatment thereof.

### Brief Description of Related Technology

Current combined antiretroviral therapies (cART) suppress viral levels in the blood but do not eradicate reservoirs of cells harboring integrated copies of HIV proviral genomes. These cells persist in part because the provirus maintains a latent state that evades the immune response and viral cytopathic effect.

Long lived latently infected cells represent a major barrier to curing HIV-1 infection. There is a significant effort to reactivate latent cellular reservoirs and allow clearing of HIV-1 infection through the cytopathic effects of viral proteins and anti-HIV-1 cytotoxic T lymphocytes (CTLs) (Deeks, 2012; Richman et al., 2009). CTLs have the potential to clear cellular reservoirs if they can be made more efficacious. However, the efficacy of major histocompatibility complex class I encoded proteins (MHC-I) restricted anti-HIV-1 CTLs is limited by the activity of HIV-1 Nef, which downmodulates MHC-I surface expression. To improve CTL recognition, the objective is to identify the ideal lead compound anti-Nef drug that reverses the inhibitory effects of Nef on MHC-I expression with low toxicity.

An objective is to generate analogs of highly potent inhibitors already identified to enhance selective anti-Nef activity in the scaffold, while reducing or eliminating off-target effects. The development of a safe and effective anti-Nef compound would allow, for the first time, the development of an approach to enhance the activity of anti-HIV-1 CTLs against residual infected cells.

Approaches that would couple latency reactivation with strategies to eradicate the infected cells—such as by design and activation of more efficacious anti-HIV cytotoxic T lymphocytes (CTLs) would facilitate clearance of these cells. Another key player is Nef, an accessory protein encoded by HIV. Because Nef inhibits the activity of anti-HIV CTLs, a potent Nef inhibitor would help achieve this goal. Nef is an accessory protein encoded by HIV that downmodulates major histocompatibility complex class I encoded proteins (MHC-I), masking infection from the host immune system and allowing HIV infected cells to persist.

A study examining HIV-1 RNA-derived nef alleles from 30 acute/early-infected individuals who participated in a clinical trial of early cART found that maximal Nef-mediated downregulation of MHC-I, but not CD4, correlated positively with post-cART replication-competent reservoir sizes (Omondi et al., 2019). Thus, drugs blocking Nef activity are expected to reduce reservoir size and potentially promote reservoir clearance. Consistent with this, a study testing the effect of small molecule inhibitors of HIV-1 Nef on the ability of autologous CD8 T cells to recognize and kill latently infected cells reported that Nef inhibition enhanced cytokine secretion and CD8 T cell-mediated elimination (Mujib et al., 2017). The reported effects were small. To date, no Nef inhibitor has achieved potent restoration of MHC-I in the presence of Nef.

### SUMMARY

The present disclosure generally relates to methods of treating HIV, to methods of inhibiting the replication of HIV viruses, to methods of reducing the amount of HIV viruses in a patient, and to compounds and compositions that can be employed for such methods.

In one aspect, the disclosure provides compounds of Formula (I) and pharmaceutically acceptable salts thereof:

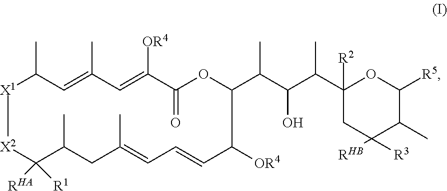

wherein

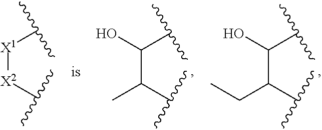

or represents a direct bond; R^{HA} and R^{HB} are H; R¹ is OH or OC(O)R⁶, or R^{HA} and R¹ together with the carbon to which they are attached form an oxo (═O) group; R² is H, OH, or OCH₃; R³ is O—C₁₋₆alkyl, O—C₁₋₆alkenyl, O—C(O)R⁷, or

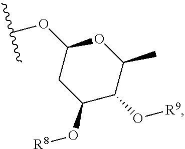

or R^{HB} and R³ together with the carbon to which they are attached form an oxo (═O) group; each R⁴ is independently H or C₁₋₆alkyl; R⁵ is C₁₋₆alkyl or C₂₋₆alkenyl; R⁶ is C₂₋₆ alkynyl or C₆₋₁₀ aryl, each optionally substituted with 1 to 3 R¹⁰; R⁷ is C₁₋₈ alkyl, C₂₋₆ alkenyl, C₂₋₆ alkynyl, or C₆₋₁₀ aryl, each optionally substituted with 1 to 3 R¹⁰; R⁸ is H or C(O)R⁶; R⁹ is H or C(O)NH₂, and R¹⁰ is C₁₋₆haloalkyl or (C₂alkylene-O)₂—C₂₋₄alkynyl, wherein the haloalkyl and alkylene are optionally substituted with a fused 3-membered heterocyloakyl ring comprising two nitrogen atoms; with the provisos that (i) when R² is OH or OCH₃ then R⁸ is not H, and R^{HB} and R³ together with the carbon to which they are attached do not form an oxo (═O) group; and (ii) when R¹ is OH, then R⁶ is not C₆₋₁₀ aryl.

In some cases, the compounds are compounds of Formula (Ia), (Ib), or (Ic):

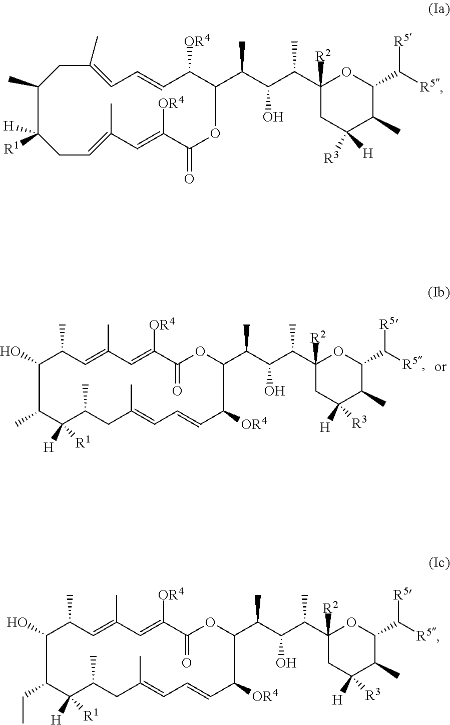

wherein (i) R^{5′} is C₂₋₅alkenyl and R^{5″} is H, or (ii) both R^{5′} and R^{5″} are C₁₋₂alkyl.

Further provided are methods of administering to a patient a safe and effective amount of a compound disclosed herein, e.g., as represented by Formulas (I), (Ia), (Ib), (Ib), or a compound of Table A, and pharmaceutically acceptable salts thereof.

Also provided are methods of modulating HIV Nef in a subject in need thereof by contacting said HIV Nef with a safe and effective amount of a compound as disclosed herein, e.g., as represented by Formulas (I), (Ia), (Ib), (Ib), or a compound of Table A, and pharmaceutically acceptable salts thereof. In some cases, modulating HIV Nef includes administering to a patient a safe and effective amount of a compound as disclosed herein e.g., as represented by Formulas (I), (Ia), (Ib), (Ib), or a compound of Table A, and pharmaceutically acceptable salts thereof.

Further provided are methods of treating an HIV Nef-associated disorder in a host by administering a safe and effective amount of a compound as disclosed herein, e.g., as represented by Formulas (I), (Ia), (Ib), (Ib), or a compound of Table A, and pharmaceutically acceptable salts thereof.

Further provided are methods of treating HIV infection in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof.

Further provided are methods of reducing an HIV reservoir in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof. Also provided are methods of eliminating an HIV reservoir in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof.

Also provided are pharmaceutical compositions comprising a compound as disclosed herein, e.g., as represented by any of Formulas (I), (Ia), (Ib), (Ib), or a compound of Table A, and pharmaceutically acceptable salts thereof, or a pharmaceutically acceptable salt thereof, and a pharmaceutically acceptable excipient, carrier, adjuvant or vehicle.

Further provided herein are uses of a compound described herein for the manufacture of a medicament for treating HIV infection in a patient, for reducing an HIV reservoir in a patient, or for eliminating an HIV reservoir in a patient.

### BRIEF DESCRIPTION OF THE DRAWINGS

FIG. 1 shows that low dose concanamycin A (CMA) restores MHC-I HLA-A2 and increases CTL killing of HIV-1 WT infected (PLAP⁺) primary T cells. Numbers are mean percent infected cell survival after a 4 hour incubation with a 2:1 effector:target (E:T) ratio of an anti-HIV-1 CTL clone recognizing Gag SL9 epitope (n=2).

FIG. 2 shows that the plecomacrolide family of compounds [left to right: CMA, Baf C1, B1, A1, and D], restore MHC-I HLA-A2 in HIV-1 infected primary T lymphocytes over a six-log range of concentrations. CMA was found to be the most potent with activity at ˜100 pM.

FIG. 3 shows that analysis of lysosome acidification based on uptake of LysoTracker® indicates there is a substantial window between concentrations of CMA necessary to inhibit Nef dependent downmodulation of MHC-I (squares) and lysosome acidification (triangles) in human primary T lymphocytes.

FIG. 4 shows that determination of the window between inhibition of Nef-mediated MHC-I downmodulation (solid line) and toxicity (dashed line) indicates superiority of CMA in HIV-1-infected primary T lymphocytes.

FIG. 5 shows LC-MS/MS generated molecular network (GnPS) comparison of crude extracts from high-priority Nef inhibition Streptomyces sp. strains 34893 (red), 5736 (orange), 39098 (blue), and 54875 (green). Circles represent individual molecular ions identified in extracts. Colors in circles represent strains identified with extracts containing those specific ions. Proportion of colors represents a comparison of ion intensity between strains. Strain 54875 lacks Baf A1.

FIG. 6 shows that Baf A1 (left lines) is a more potent Nef inhibitor of MHC-1 downmodulation in HIV-1 infected primary T lymphocytes than the most potent previously published Nef inhibitor (B9) (right lines) (Mujib et al., 2017). The B9 (Sigma) structure was confirmed by NMR.

FIG. 7 shows methodology for high-throughput derivatization and testing of Baf A1-and/or CMA amide analogs. (1) Initial activation of Baf A1 (and/or CMA) as NHS-esters, followed by (2) coupling with a library of primary amines in a 96 well plate format. These are then dried and submitted for high-throughput testing in the whole-cell Nef-inhibition assay (3). High priority targets are identified and scaled up further (4).

FIG. 8 shows that low dose CMA (0.5 nM) increases CTL killing of HIV-1 WT infected primary T cells to that observed for HIV-1 lief infected cells. Mean +/− standard deviation is shown n=2. E:T, effector:target.

FIG. 9 shows that CMA selectively reverses Nef-dependent degradation of MHC-I in sorted HIV-1 infected primary T lymphocytes. Monoclonal antibody HC-10 recognizes almost all MHC-I HLA-B and some HLA-A heavy chains.

FIG. 10 shows CMA labeling approaches for target identification, illustrating (A) proposed structures, and positions for derivatization, introducing a photoaffinity label, and a selective chemical handle for coupling with biotin; (8) proposed proteomics approach for target identification via formation of a target-CMA-biotin conjugate; (i) cell lysate treated with doubly-labeled CMA; (ii) sample irradiated with UV-light, coupling CMA to target proteins; (iii) sample coupled to activated-biotin using reactive biotin reporter tag; (iv) targets captured via biotin affinity chromatography (figure adapted from Smith et al., 2015); (C) proposed coupling of an azido-CMA derivative with alkyne-resin to yield a CMA coupled resin for use in CMA-affinity chromatography.

FIG. 11 shows a comparison of the 72 hour viability TC₅₀ (triangles), 24 Nef activity IC₅₀ (circles), and 24 hr Lysotracker IC₅₀ (diamonds) for Bafilomycin A₁ and its derivatives. A concentration of 1000 nM was set as the activity thresholds for the assays. Dots represent biological replicates with independent donors.

FIG. 12 shows a comparison of the 72 hour viability TC₅₀ (triangles), 24 Nef activity IC₅₀ (circles), and 24 hr Lysotracker IC₅₀ (diamonds) for Bafilomycin A₁ and its proteomic-related derivatives. A concentration of 1000 nM was set as the activity thresholds for the assays. Dots represent biological replicates with independent donors.

FIG. 13 shows a comparison of the 72 hour viability TC₅₀ (triangles), 24 Nef activity IC₅₀ (circles), and 24 hr Lysotracker IC₅₀ (diamonds) for Concanamycins A-C and their derivatives. A concentration of 1000 nM was set as the activity thresholds for the assays. Dots represent biological replicates with independent donors.

FIG. 14 shows a comparison of the 72 hour viability TC₅₀ (triangles), 24 Nef activity IC₅₀ (circles), and 24 hr Lysotracker IC₅₀ (diamonds) for Concanamycin A-C and their proteomic-related derivatives. A concentration of 1000 nM was set as the activity thresholds for the assays. Dots represent biological replicates with independent donors.

### DETAILED DESCRIPTION

Provided herein are analogs of plecomacrolides, such as analogs of Bafilomycins (Bafs), Concanamycins, Leucanicidin, and Virustomycin. In embodiments, the analogs of plecomacrolides include analogs of Bafilomycin A1 (Baf A1), PC-766B, Leucanicidin, Concanamycin A (CMA), Concanamycin B, Concanamycin C, and Virustomycin.

Also provided are methods of modulating HIV Nef in a subject in need thereof comprising administering to the subject an analog of one or more Bafilomycin A1, PC-766B, Leucanicidin, Concanamycin A, Concanamycin B, Concanamycin C, and Virustomycin in an amount effective to inhibit HIV Nef in the subject. Further provided are methods of treating a HIV Nef-associated disorder in a subject in need thereof comprising administering a therapeutically effective amount of an analog of one or more of Bafilomycin A1, PC-766B, Leucanicidin, Concanamycin A, Concanamycin B, Concanamycin C, and Virustomycin. In some cases, the Nef-associated disorder is HIV infection. In some cases, the HIV infection is HIV-1 infection. In some cases, the HIV-1 infection is infection with HIV subtype A, B, C, D, E, F, G, H, I, J, K, L, or a recombination thereof. In some cases, treatment of HIV infection comprises reducing an HIV reservoir in a host. In some cases, treatment of HIV infection comprises eliminating an HIV reservoir in a host.

A screen of natural product extracts was preformed and identified a number of related compounds that potentially restored surface expression of MHC-I in the presence of NEF with potencies that differed by six-orders of magnitude in human primary cells. While a known target of these compounds is vacuolar ATPase (V-ATPase), which is necessary for lysosomal function, Nef inhibition was separable from effects on the lysosome. It has been previously found that concanamycin A restored MHC-I to the surface of Nef-expressing cells at concentrations that did not interfere with lysosomal acidification and demonstrated no observable toxicity in primary cell cultures. The prior work with CMA has identified a novel activity of this family of natural products at lower concentrations (pM) than are needed to affect lysosome function (nM).

It has advantageously been determined that analogs that isolate the Nef inhibitory activity of these molecules have great potential as a safe anti-Nef drug. These potent inhibitors are improved through isolation of the anti-Nef effect from off-target activities to identify a leading drug candidate for development. The analogs herein are Nef inhibitors which can facility the goal of CTL mediated clearance of reactivated latent reservoirs of HIV in virally suppressed people. A rational structural-activity relationship approach to the development of analogs is utilized by determining the mechanism by which the inhibitors disrupt Nef-mediated MHC-I downmodulation.

The analogs herein are Nef inhibitors. They represent a new class of drugs that can increase efficacy of the immune response by enhancing CTL recognition and killing of HIV-1 infected cells that have been reactivated from latency. In embodiments, the analogs disclosed herein can be added to cART cocktails for enhanced immune clearance, and eradication of viral reservoirs in the treatment of HIV. In combination with cART, latency antagonists and possibly a strategy to generate more efficacious CTLs, such compounds will increase the likelihood that cellular reservoirs will be eradicated by the host immune response.

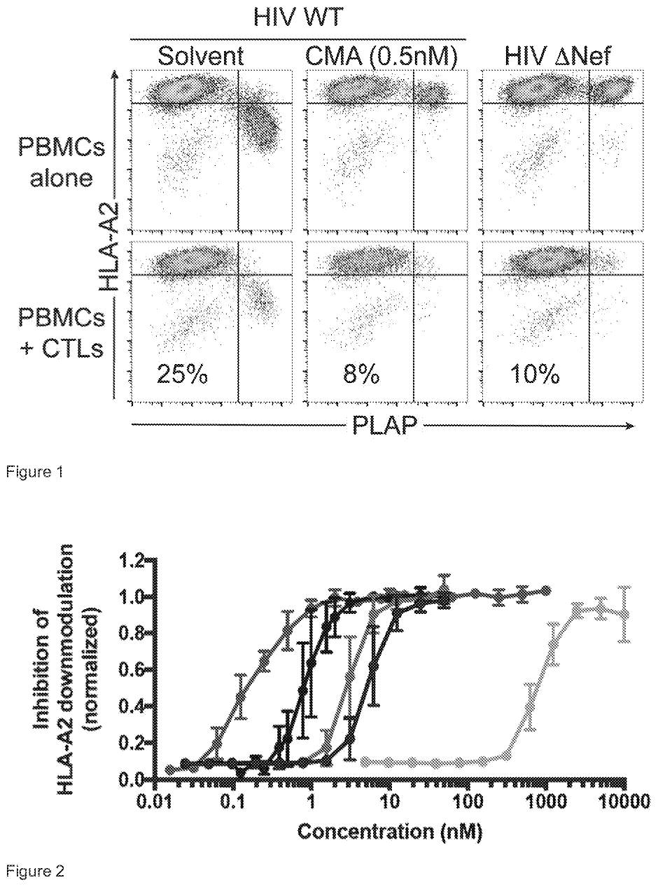

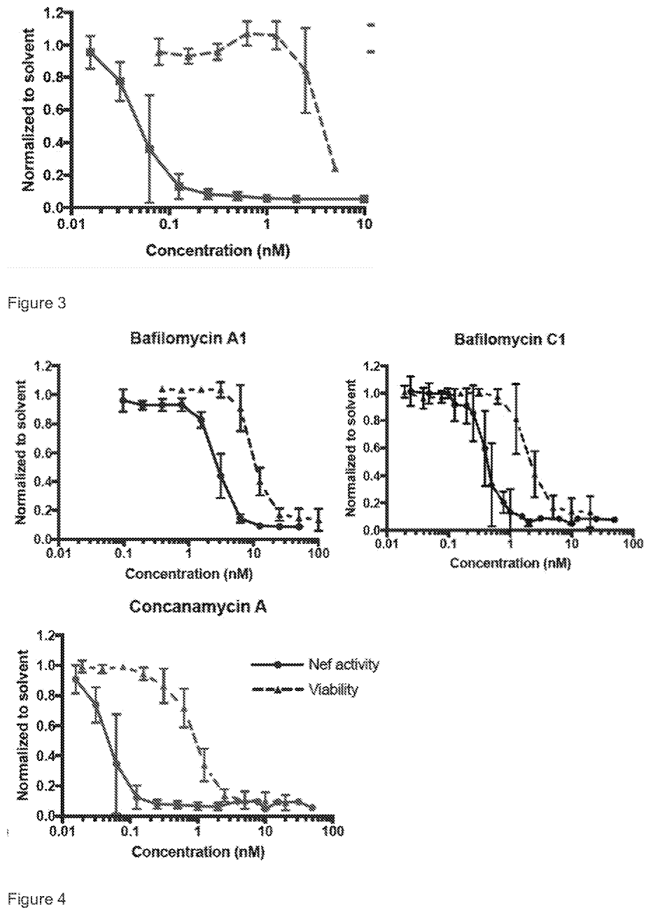

Analogs disclose herein can be derived from Bafilomycins (Bafs) and/or concanamycin A (CMA). The analogs based on these scaffolds can have one or more of high potency and low toxicity. A family of macrocyclic natural products that differ in discrete functionality resulting in over six-log NEF inhibitory potency range has been identified (FIG. 2). A known activity of these molecules that causes toxicity (V-ATPase inhibition) is distinct and separable from Nef inhibition (FIGS. 3 and 4). Iterative rounds of Baf/CMA modification and testing can lead to optimization of the NEF inhibition of the analogs of the disclosure.

A family of natural product compounds was identified that restores surface expression of MHC-I to HIV-1 infected cells and enhances CTL killing to levels observed in the absence of Nef [concanamycin A (CMA) in FIG. 1]. Surface expression was restored over a six-log range of concentrations extending to the picomolar range (FIG. 2). The data demonstrated that the inhibitors identified were far more potent than any previously published Nef inhibitors claiming to reverse MHC-I downmodulation by HIV-1 Nef (Mujib et al., 2017). While the compounds identified were known to be toxic via inhibition of lysosomal acidification by vacuolar ATPase (V-ATPase), the data herein demonstrates that this mechanism of action is dispensable for inhibition of MHC-I downmodulation by Nef (FIG. 3). Moreover, it is shown that inhibition of Nef occurs at amounts of drug that are nontoxic in assays herein, even under conditions of prolonged continuous incubation over three days (FIG. 4). The analogs disclosed herein have reduced effects on V-ATPase to create a safe and effective anti-Nef small molecule therapeutic agent.

Plecomacrolides are a family of natural products featuring a 16-18 membered macrolactone. Previous work has shown that select members of this family function as inhibitors of V-ATPase, which is needed for acidification of lysosomes and their proper degradative function. Because Nef disrupts MHC-I by targeting it into the endolysosomal pathway, it is possible that this activity of plecomacrolides explains their Nef inhibitory effect. However, without intending to be bound by theory, it is instead believed that inhibition of Nef by plecomacrolides occurs through a previously undescribed target of these compounds. Some examples of alternative molecular targets for other natural products and synthetic drugs have been recently described (Effenberger et al., 2017). Members of the plecomacrolide family, including Baf A1, have been considered potential therapeutics for osteoporosis and cancer. However, potentially limiting features of these secondary metabolites include toxicity, availability and cost. One study examining the efficacy of Baf A1 against leukemia determined the maximum tolerated single dose in mice. When administered to C57BL/6J mice by intraperitoneal injection daily over three days at doses of 10 mg/kg, the mice maintained a normal weight and showed no evidence of liver toxicity but at 25 mg/kg the mice experienced increases in liver enzymes indicative of liver damage and 30% weight loss within 12 days of injection (Yuan et al., 2015). In another study examining toxicity of Baf A1 and CMA, eleven-week inbred C57BL/6J and BALB/c mice were exposed to five consecutive doses at 12 mg/kg body weight (an amount previously observed to cause a 50% decrease in kidney V-ATPase without altering the weight or protein content of the kidneys). Overall body weight was not affected by any of the treatments. Moreover, Baf A1 and CMA did not cause significant variations in random blood glucose but a decrease in pancreatic islet cell size was noted (Hettiarachchi et al., 2006). These in vivo studies indicate there is a relatively narrow therapeutic window between doses capable of inhibiting V-ATPase and doses inducing deleterious effects. Based on preliminary data showing that CMA inhibits Nef at concentrations ˜10-fold lower than those needed to inhibit lysosome acidification (FIG. 3), concentrations of the analogs herein that do not inhibit V-ATPase are safe and effective Nef inhibitors. Thus, the analogs herein are new Baf and/or CMA-based structure that maximizes Nef inhibition, while minimizing V-ATPase activity/toxicity.

Analogs disclosed herein are tested for efficacy, toxicity, effect on lysosome inhibition and enhancement of CTL killing. Preliminary data for four different BAFs (Bafs A1, B1, C1, D) and CMA demonstrate variations in these activities depending on discrete chemical functionality. Toxicity can be tested with MTT assays. Efficacy can be tested by dose-dependent reversal of NEF downmodulation across a range of activities. Lysosomal acidification can be analyzed by dose dependent reversal of LysoTracker® uptake.

Without intending to be bound by theory, it is believed that Baf/CMA natural products disrupt MHC-I downmodulation at a step prior to lysosomal degradation. It is believed that this occurs by re-routing MHC-I back to its normal trafficking pathway, potentially disrupting NEF-dependent trafficking complexes. This hypothesis is supported by preliminary data showing concentrations of CMA that do not affect lysosomal pH fully inhibit the ability of Nef to disrupt MHC-I trafficking (FIG. 3). Assays have been developed to measure Nef-dependent MHC-I trafficking to assess how they are altered in the presence of CMA and the analogs herein that show similar or higher potency against Nef). Two chemical biology approaches can also be used to identify all potential protein targets in primary T lymphocytes; 1) affinity chromatography using CMA as “bait” molecules and 2) small molecule target capture using an affinity labeled form of CMA or potent Baf analog. The analogs can be tested to assess NEF-dependent MHC-I trafficking and alternation in the presence of CMA and the analogs disclosed herein. Complementary unviased affinity labeling approaches can be used to identify CMA binding partners in primary T-lymphocytes to characters novel CMA targets proposed to inhibit NEF.

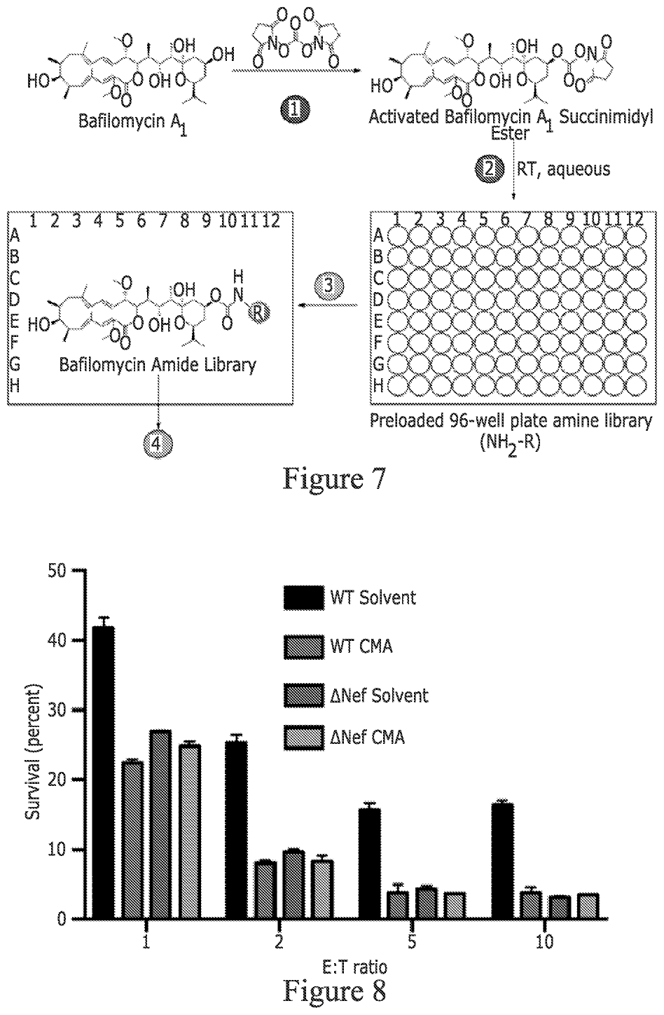

Previous structure activity relationship (SAR) studies have been conducted on CMA (Dröse et al., 2001; Ingenhorst et al., 2001). This work showed that reduction of the hemiketal (position-21, FIG. 7) to the corresponding deoxy-analog had minimal effect on V-ATPase inhibition while dramatically improving the stability of the compounds (Dröse et al., 2001; Ingenhorst et al., 2001). The corresponding Baf analog was generated and found it to have similar anti-Nef activity to the natural compound (data not shown).

A CMA deoxy-analog was generated, and derivatization was performed on these molecules. Without wishing to be bound by any particular theory, this can improve stability for both subsequent derivatizations and preclinical testing.

### Analogs of Plecomacrolides

The disclosure provides analogs of plecomacrolides. In some cases, the disclosure herein provides analogs for plecomacrolides having 16-18 membered rings. The plecomacrolides can undergo a modification to a plecomacrolide scaffold to provide an analog of plecomacrolides. The plecomacrolide scaffolds can include, but are not limited to, Bafilomycin A1, PC-766B, Leucanicidin, Concanamycin A, Concanamycin B, Concanamycin C, and Virustomycin. In embodiments, the modifications of the plecomacrolide scaffolds can include acylation, glycosylation, and amidation via succinimidyl ester activation.

In embodiments, the analogs of plecomacrolides have a structure selected from:

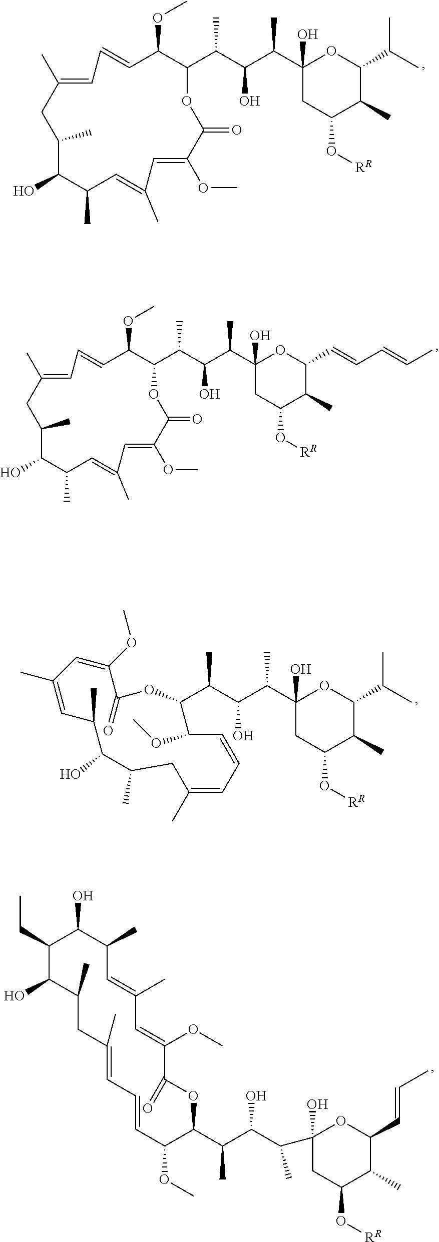

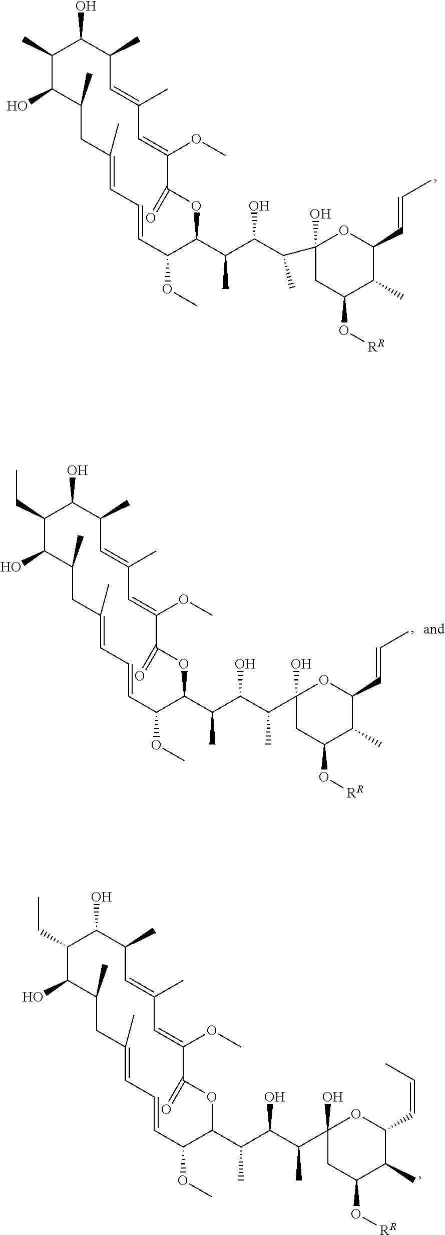

wherein each R^{R} is independently C(O)R^{R6}, a sugar moiety, or C(O)NHR^{R1}; each R^{R6} is independently C₁₋₆ alkyl or a sugar moiety; each R^{R1} is independently a C₁₋₂₀ alkyl, C₂₋₁₂ alkenyl, C₅-C₈ cycloalkyl, C₂₋₈ heterocycloalkyl, or Ar¹; and each Ar¹ is independently selected from C₆-C₂₂ aryl or a 5-12 membered heteroaryl comprising from 1 to 3 ring heteroatoms selected from O, N, and S.

In some cases, R^{R} is C(O)R^{R6}. In embodiments, R^{R6} is C₁₋₆ alkyl. In embodiments, R^{R6} is a sugar moiety. A sugar can be a pentose, hexose, heptose, or an amino sugar (e.g., aminopentose, aminohexose, aminoheptose, or a neuraminic acid), for example. Some contemplated sugars include, but are not limited to, allose, altrose, glucose, mannose, gulose, idose, galactose, talose, sialic acid, glucosamine, galactosamine, fructose, arabinose, dextrose, sorbose, psicose, tagatose, sucrose, lactose, maltose, trehalose, cellobiose, chitobiose, lactulose, kojibiose, nigerose, isomaltose, sophorose, laminaribose, gentiobiose, turanose, matlulose, plaltinose, gentiobiulose, mannobiose, melibiose, melibiulose, rutinose, rutinulose, N-acetylglucosamine, fucose, N-acetylneuraminic acid, sialic acid, xylobiose, ribose, rhamnose, xylose, cladinose, mycinose, javose, 2-deoxy-β-D-rhamnose, and the like. Contemplated amino sugars include desosamine, mycaminose, and the like. For the avoidance of doubt, the terms “carbohydrate,” “sugar,” and “saccharide” are all used interchangeably.

In some cases, R^{R} is a sugar moiety. In some cases, R^{R} is an amino sugar moiety.

In some embodiments, R^{R} is C(O)NHR^{R9}, wherein R^{R9} is C₁₋₂₀ alkyl, C₂₋₁₂ alkenyl, C₅-C₈ cycloalkyl, C₂₋₁₂ heterocycloalkyl, or Ar¹. In some cases, R^{R9} is C₁₋₂₀ alkyl. In some cases, R^{R9} is C₁₋₆ alkyl. In some cases, R^{R9} is C₂₋₁₂ alkenyl. In some cases, R^{R9} is C₅-C₈ cycloalkyl. In some cases, R^{R9} is Ar¹. In some cases, R^{R9} is Ph. In some cases, R^{R9} is C₂₋₁₂ heterocycloalkyl.

### Compounds

The present disclosure provides compounds of Formula I, and pharmaceutically acceptable salts thereof:

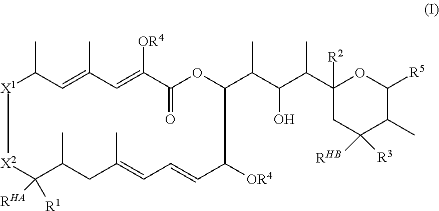

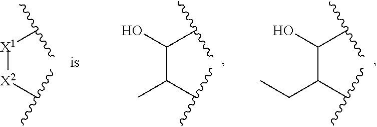

or represents a direct bond;
R^{HA} and R^{HB} are H;
R¹ is OH or OC(O)R⁶,
   or R^{HA} and R¹ together with the carbon to which they are attached form an oxo (═O) group;
R² is H, OH, or OCH₃;
R³ is O—C₁₋₆alkyl, O—C₁₋₆alkenyl, O—C(O)R⁷, or

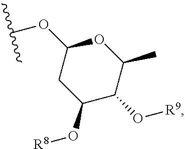

or R^{HB} and R³ together with the carbon to which they are attached form an oxo (═O) group;
each R⁴ is independently H or C₁₋₆alkyl;
R⁵ is C₁₋₆alkyl or C₂₋₆alkenyl;
R⁶ is C₂₋₆ alkynyl or C₆₋₁₀ aryl, each optionally substituted with 1 to 3 R¹⁰;
R⁷ is C₁₋₈ alkyl, C₂₋₆ alkenyl, C₂₋₆ alkynyl, or C₆₋₁₀ aryl, each optionally substituted with 1 to 3 R¹⁰;
R⁸ is H or C(O)R⁶;
R⁹ is H or C(O)NH₂, and
R¹⁰ is C₁₋₆haloalkyl or (C₂alkylene-O)₂—C₂₋₄alkynyl, wherein the haloalkyl and alkylene are optionally substituted with a fused 3-membered heterocyloakyl ring comprising two nitrogen atoms;
   with the provisos that (i) when R² is OH or OCH₃ then R⁸ is not H, and R^{HB} and R³ together with the carbon to which they are attached do not form an oxo (═O) group, and (ii) when R¹ is OH, then R⁶ is not C₆₋₁₀ aryl.

In some cases, the compound has a structure of Formula (Ia), (Ib), or (Ic):

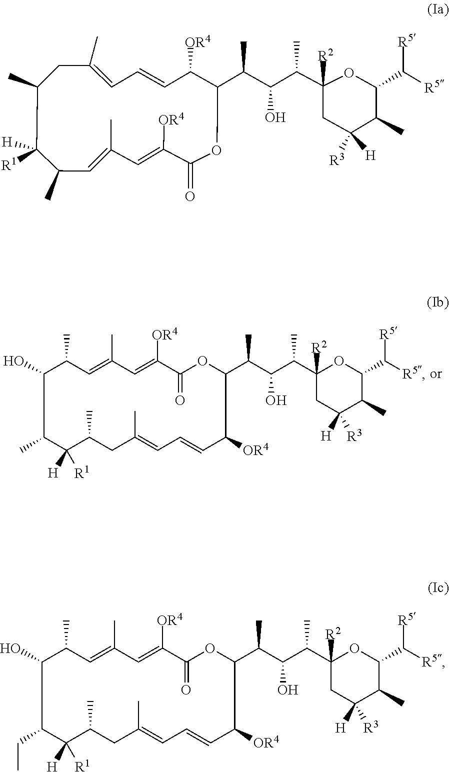

wherein (i) R^{5′} is C₂₋₅alkenyl and R^{5″} is H, or (ii) both R^{5′} and R^{5″} are C₁₋₂alkyl. In some cases, the compound has a structure of Formula Ia

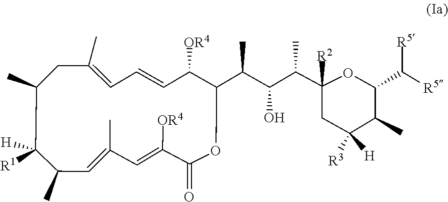

In some cases, the compound has a structure of Formula Ib:

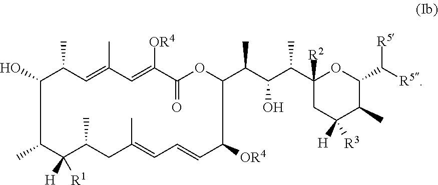

In some cases, the compound has a structure of Formula Ic:

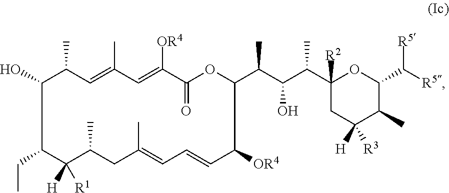

In some cases, R^{HA} and R^{HB} are both H. In some cases, R^{HA} is H. In some cases, R^{HB} is H.

In some cases, R¹ is OH. In some cases, R¹ is OC(O)R⁶. In some cases, R^{HA} and R¹ together with the carbon to which they are attached form an oxo (═O) group. In some cases, R¹ is

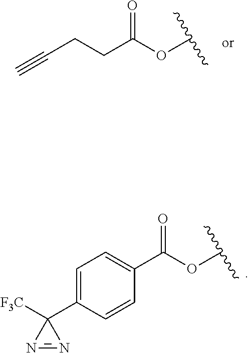

In some cases, R² is H. In some cases, R² is OH. In some cases, R² is OCH₃.

In some cases, R³ is O—C₁₋₆alkyl or O—C₁₋₆alkenyl. In some cases, R³ is OCH₃ or

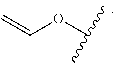 [SMILES: *OC=C]

In some cases, R³ is O—C₁₋₆alkyl. In some cases, R³ is OCH₃. In some cases, R³ is O—C₁₋₆alkenyl. In some cases, R³ is

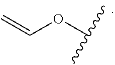 [SMILES: *OC=C]

In some cases, R³ is O—C(O)R⁷. In some cases, R³ is

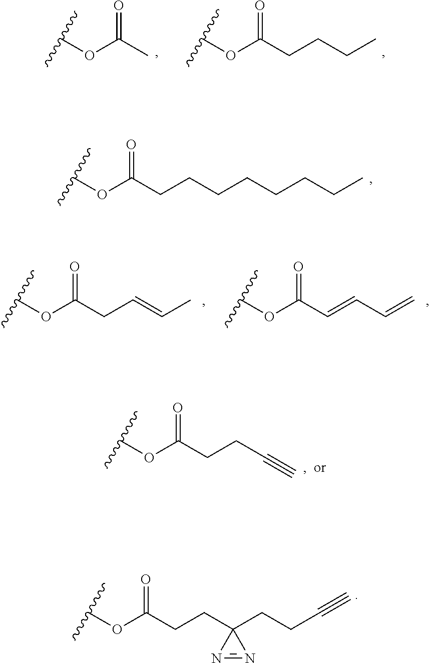

In some cases, R³ is

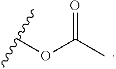 [SMILES: *OC(C)=O]

In some cases, R³ is

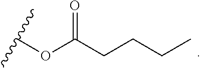 [SMILES: *OC(=O)CCCC]

In some cases, R³ is

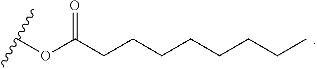 [SMILES: *OC(=O)CCCCCCCC]

In some cases, R³ is

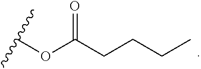 [SMILES: *OC(=O)CCCC]

In some cases, R³ is

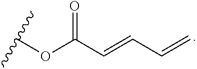 [SMILES: *OC(=O)/C=C/C=C]

In some cases, R³ is

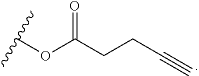 [SMILES: *OC(=O)CCC#C]

In some cases, R³ is

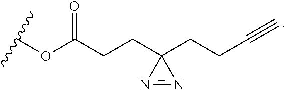 [SMILES: *OC(=O)CCC1(CCC#C)N=N1]

In some cases, R³ is

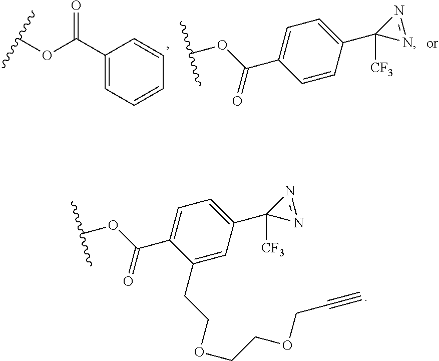

In some cases, R³ is

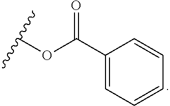 [SMILES: *OC(=O)c1ccccc1]

In some cases, R³ is

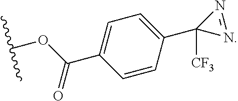 [SMILES: *OC(=O)c1ccc(C2(C(F)(F)F)N=N2)cc1]

In some cases, R³ is

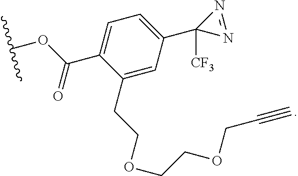 [SMILES: *OC(=O)c1ccc(C2(C(F)(F)F)N=N2)cc1CCOCCOCC#C]

In some cases, R³ is

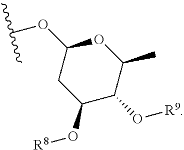

In some cases, R⁸ is H. In some cases, R⁸ is C(O)R⁶. In some cases, R⁸ is

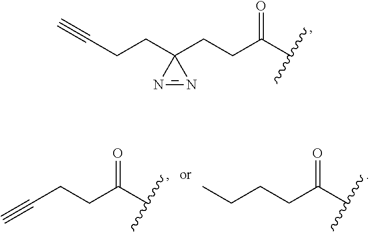

In some cases, R⁸ is

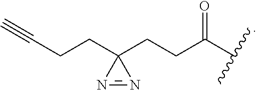 [SMILES: *C(=O)CCC1(CCC#C)N=N1]

In some cases, R⁸ is

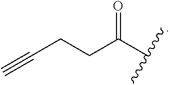 [SMILES: *C(=O)CCC#C]

In some cases, R⁸ is

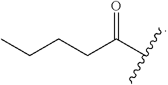 [SMILES: *C(=O)CCCC]

In some cases, R⁹ is H. In some cases, R⁹ is C(O)NH₂. In some cases, R^{HB} and R³ together with the carbon to which they are attached form an oxo (═O) group. In some cases, R³ is

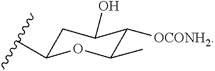

In some cases, at least one R⁴ is C₁₋₆alkyl. In some cases, each R⁴ is C₁₋₆alkyl. In some cases, at least one R⁴ is methyl. In some cases, each R⁴ is methyl.

In some cases, R⁵ is C₁₋₆alkyl. In some cases, R⁵ is C₃alkyl. In some cases, R⁵ is isopropyl. In some cases, R⁵ is C₂₋₆alkenyl. In some cases, R⁵ is

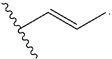 [SMILES: */C=C/C]

In some cases, R⁵ is

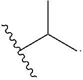 [SMILES: *C(C)C]

In some cases, R^{5′} is C₁₋₅alkyl. In some cases, In some cases, R^{5′} is methyl. In some cases, R^{5′} is C₂₋₅alkenyl. In some cases, R^{5′} is C₃alkenyl. In some cases, R^{5′} is allyl. In some cases, R^{5″} is H. In some cases, R^{5″} is C₁₋₅alkyl. In some cases, R^{5″} is methyl. In some case, R^{5′} is C₂₋₅alkenyl and R^{5″} is H. In some cases, In some case, R^{5′} is C₂alkenyl and R^{5″} is H. In some cases, both R^{5′} and R^{5″} are C₁₋₂alkyl. In some cases, R^{5′} and R^{5″} are methyl.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is drawn specifically.

Therefore, single stereochemical isomers as well as enantiomeric, diastereomeric, cis/trans, conformational, and rotational mixtures of the present compounds are within the scope of the disclosure.

Unless otherwise indicated, all tautomeric forms of the compounds described herein are within the scope of the disclosure.

Additionally, unless otherwise indicated, structures depicted herein are also meant to include compounds that differ only in the presence of one or more isotopically enriched atoms. Discussion of an element is intended to include all isotopes of that element. For example, a substituent shown as a hydrogen includes where that hydrogen is in the deuterium or tritium isotope form, and a carbon atom can be present as a ¹³C— or ¹⁴C— carbon isotope.

It is understood that selections of values of each variable are those that result in the formation of stable or chemically feasible compounds.

Also provided are compounds listed in Table A, and pharmaceutically acceptable salts thereof:

TABLE A

| Compound | |
|---|---|
| No. | Structure |
| 1 | 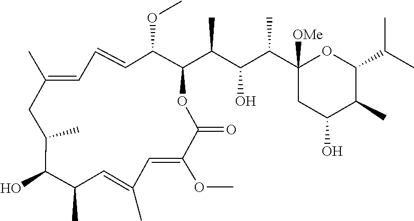 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(OC)C[C@@H](O)[C@H](C)[C@@H](C(C)C)O2)OC1=O] |
| 2 | 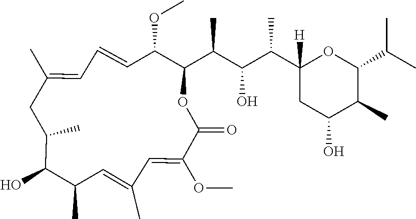 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](O)[C@H](C)[C@@H](C(C)C)O2)OC1=O] |
| 3 | 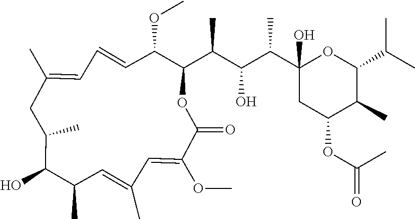 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(C)=O)[C@H](C)[C@@H](C(C)C)O2)OC1=O] |
| 4 | 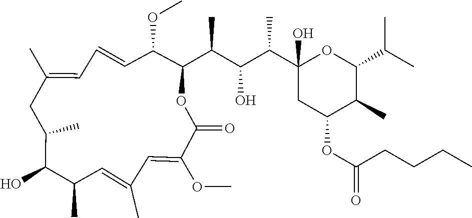 [SMILES: CCCCC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 5 | 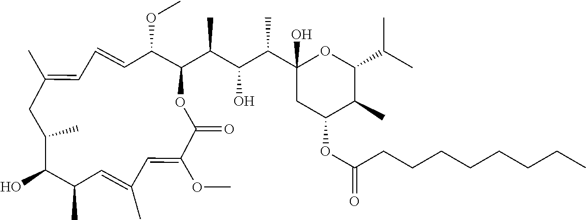 [SMILES: CCCCCCCCC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 6 | 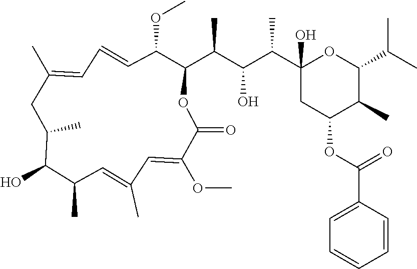 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)c3ccccc3)[C@H](C)[C@@H](C(C)C)O2)OC1=O] |
| 7 | 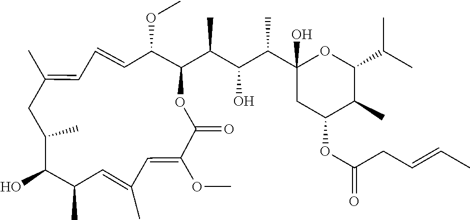 [SMILES: C/C=C/CC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 8 | 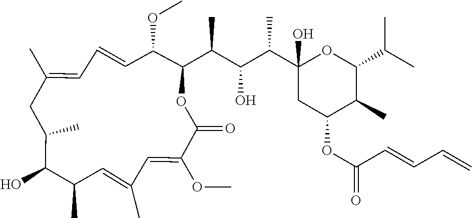 [SMILES: C=C/C=C/C(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 9 | 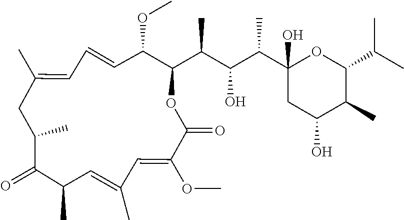 [SMILES: CO/C1=C\C(C)=C\[C@@H](C)C(=O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O] |
| 10 | 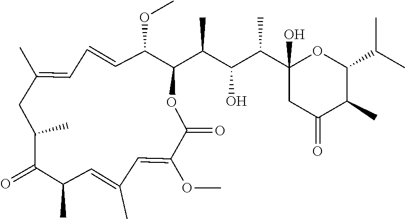 [SMILES: CO/C1=C\C(C)=C\[C@@H](C)C(=O)[C@@H](C)C/C(C)=C/C=C/[C@H](OC)[C@@H]([C@@H](C)[C@@H](O)[C@H](C)[C@@]2(O)CC(=O)[C@H](C)[C@@H](C(C)C)O2)OC1=O] |
| 11 | 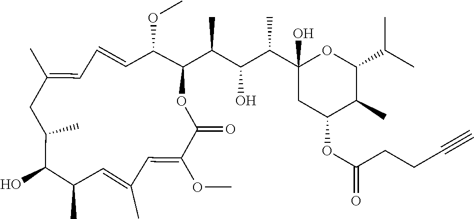 [SMILES: C#CCCC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 12 | 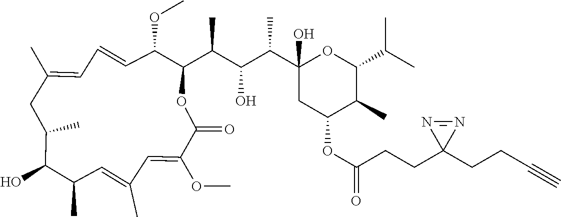 [SMILES: C#CCCC1(CCC(=O)O[C@@H]2C[C@](O)([C@@H](C)[C@H](O)[C@H](C)[C@H]3OC(=O)/C(OC)=C/C(C)=C/[C@@H](C)[C@@H](O)[C@@H](C)C/C(C)=C/C=C/[C@@H]3OC)O[C@H](C(C)C)[C@H]2C)N=N1] |
| 13 | 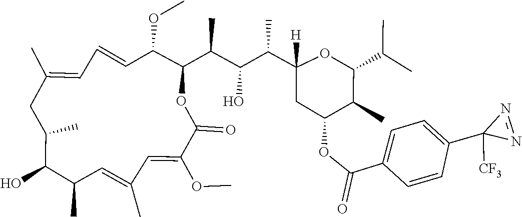 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](OC(=O)c3ccc(C4(C(F)(F)F)N=N4)cc3)[C@H](C)[C@@H](C(C)C)O2)OC1=O] |
| 14 | 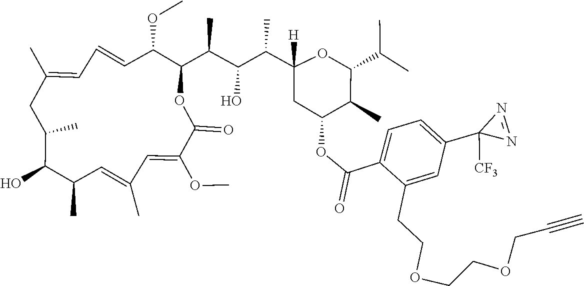 [SMILES: C#CCOCCOCCc1cc(C2(C(F)(F)F)N=N2)ccc1C(=O)O[C@@H]1C[C@H]([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 15 | 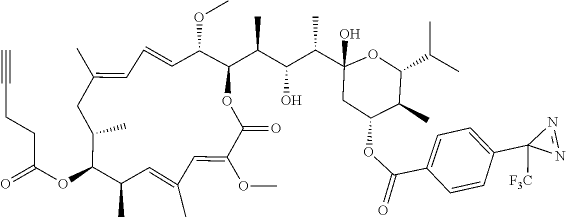 [SMILES: C#CCCC(=O)O[C@@H]1[C@H](C)/C=C(C)/C=C(\OC)C(=O)O[C@H]([C@@H](C)[C@@H](O)[C@H](C)[C@@]2(O)C[C@@H](OC(=O)c3ccc(C4(C(F)(F)F)N=N4)cc3)[C@H](C)[C@@H](C(C)C)O2)[C@@H](OC)/C=C/C=C(\C)C[C@@H]1C] |
| 16 | 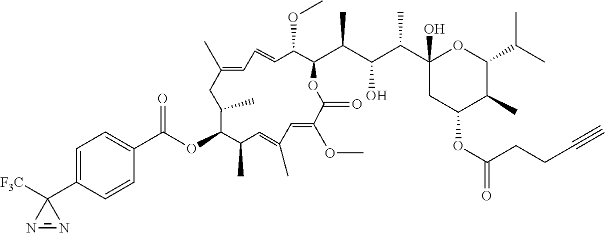 [SMILES: C#CCCC(=O)O[C@@H]1C[C@](O)([C@@H](C)[C@H](O)[C@H](C)[C@H]2OC(=O)/C(OC)=C/C(C)=C/[C@@H](C)[C@@H](OC(=O)c3ccc(C4(C(F)(F)F)N=N4)cc3)[C@@H](C)C/C(C)=C/C=C/[C@@H]2OC)O[C@H](C(C)C)[C@H]1C] |
| 17 | 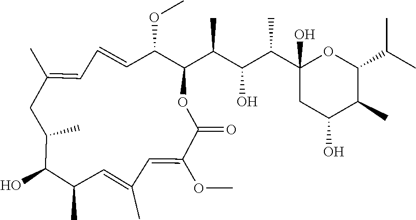 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O] |
| 18 | 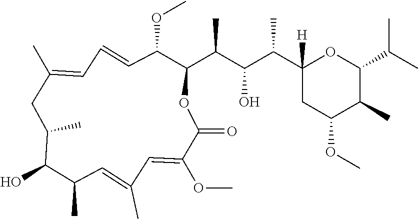 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](OC)[C@H](C)[C@@H](C(C)C)O2)OC1=O] |
| 19 | 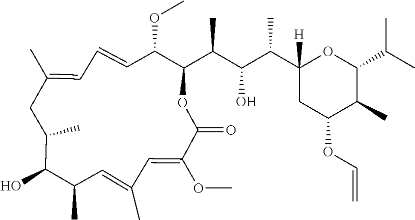 [SMILES: C=CO[C@@H]1C[C@H]([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 20 | 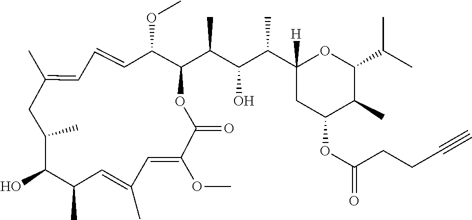 [SMILES: C#CCCC(=O)O[C@@H]1C[C@H]([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C] |
| 21 | 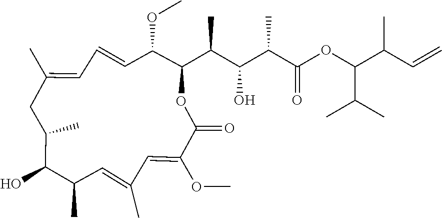 [SMILES: C=CC(C)C(OC(=O)[C@@H](C)[C@H](O)[C@H](C)[C@H]1OC(=O)/C(OC)=C/C(C)=C/[C@@H](C)[C@@H](O)[C@@H](C)C/C(C)=C/C=C/[C@@H]1OC)C(C)C] |
| 22 | 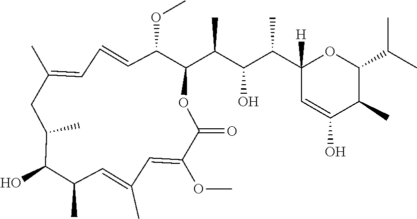 |
| 23 | 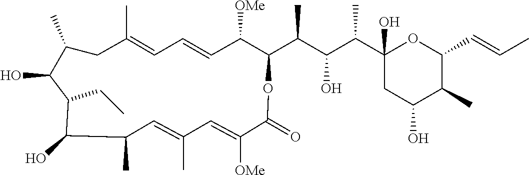 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O)[C@@H]1C] |
| 24 | 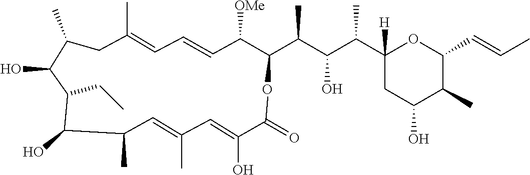 [SMILES: C/C=C/[C@H]1O[C@@H]([C@@H](C)[C@H](O)[C@H](C)[C@H]2OC(=O)/C(O)=C/C(C)=C/[C@@H](C)[C@@H](O)[C@@H](CC)[C@@H](O)[C@H](C)C/C(C)=C/C=C/[C@@H]2OC)C[C@@H](O)[C@@H]1C] |
| 25 | 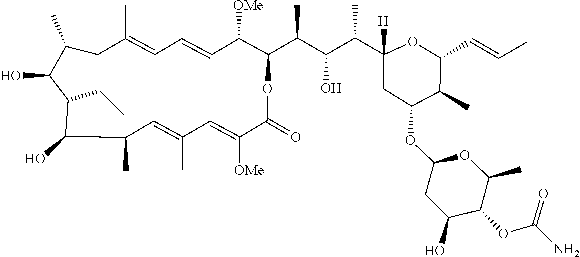 [SMILES: C/C=C/[C@H]1O[C@@H]([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](OC(N)=O)[C@H](C)O2)[C@@H]1C] |
| 26 | 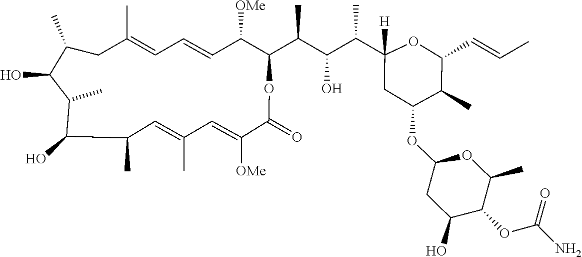 [SMILES: C/C=C/[C@H]1O[C@@H]([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](O)[C@@H](OC(N)=O)[C@H](C)O2)[C@@H]1C] |
| 27 | 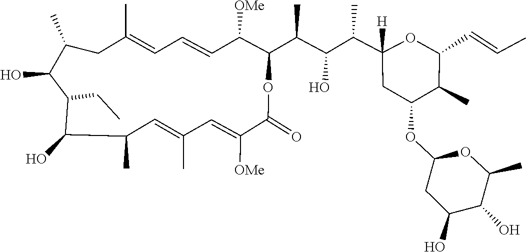 [SMILES: C/C=C/[C@H]1O[C@@H]([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C] |
| 28 | 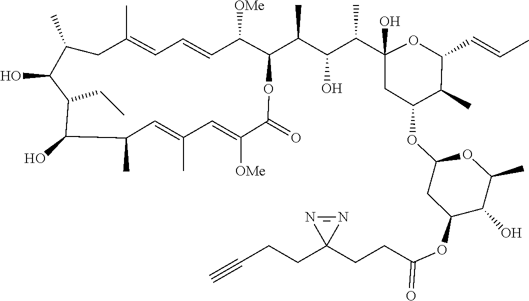 [SMILES: C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](CC)[C@@H](O)[C@H](C)C/C(C)=C/C=C/[C@@H]4OC)O[C@H](/C=C/C)[C@H]3C)O[C@@H](C)[C@@H]2O)N=N1] |
| 29 | 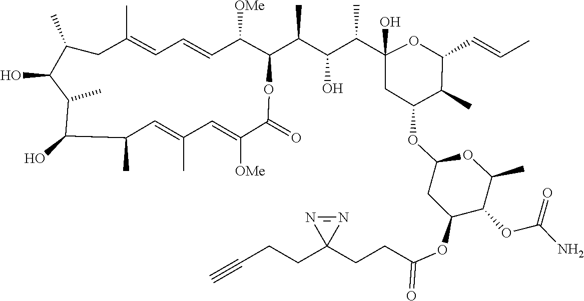 [SMILES: C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](C)[C@@H](O)[C@H](C)C/C(C)=C/C=C/[C@@H]4OC)O[C@H](/C=C/C)[C@H]3C)O[C@@H](C)[C@@H]2OC(N)=O)N=N1] |
| 30 | 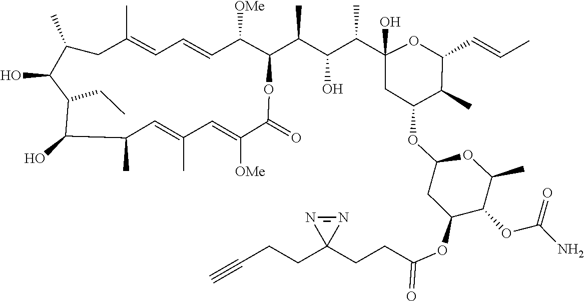 [SMILES: C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](CC)[C@@H](O)[C@H](C)C/C(C)=C/C=C/[C@@H]4OC)O[C@H](/C=C/C)[C@H]3C)O[C@@H](C)[C@@H]2OC(N)=O)N=N1] |
| 31 | 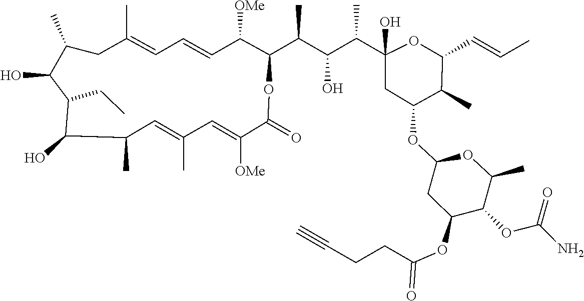 [SMILES: C#CCCC(=O)O[C@H]1C[C@@H](O[C@@H]2C[C@](O)([C@@H](C)[C@H](O)[C@H](C)[C@H]3OC(=O)/C(OC)=C/C(C)=C/[C@@H](C)[C@@H](O)[C@@H](CC)[C@@H](O)[C@H](C)C/C(C)=C/C=C/[C@@H]3OC)O[C@H](/C=C/C)[C@H]2C)O[C@@H](C)[C@@H]1OC(N)=O] |
| 32 | 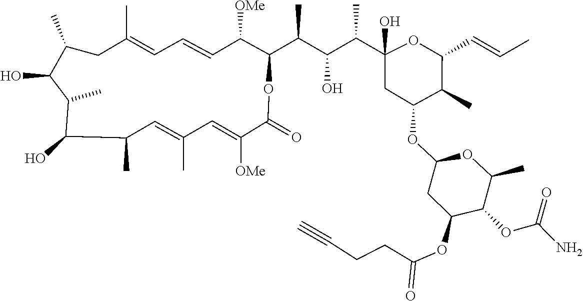 [SMILES: C#CCCC(=O)O[C@H]1C[C@@H](O[C@@H]2C[C@](O)([C@@H](C)[C@H](O)[C@H](C)[C@H]3OC(=O)/C(OC)=C/C(C)=C/[C@@H](C)[C@@H](O)[C@@H](C)[C@@H](O)[C@H](C)C/C(C)=C/C=C/[C@@H]3OC)O[C@H](/C=C/C)[C@H]2C)O[C@@H](C)[C@@H]1OC(N)=O] |
| 33 | 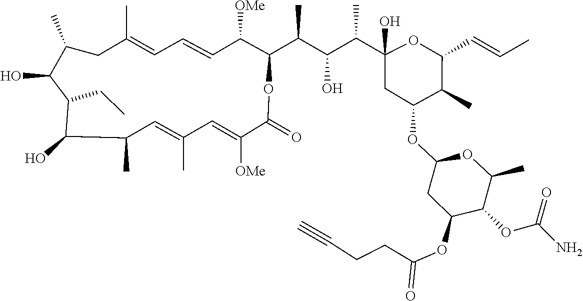 [SMILES: C#CCCC(=O)O[C@H]1C[C@@H](O[C@@H]2C[C@](O)([C@@H](C)[C@H](O)[C@H](C)[C@H]3OC(=O)/C(OC)=C/C(C)=C/[C@@H](C)[C@@H](O)[C@@H](CC)[C@@H](O)[C@H](C)C/C(C)=C/C=C/[C@@H]3OC)O[C@H](/C=C/C)[C@H]2C)O[C@@H](C)[C@@H]1OC(N)=O] |
| 34 | 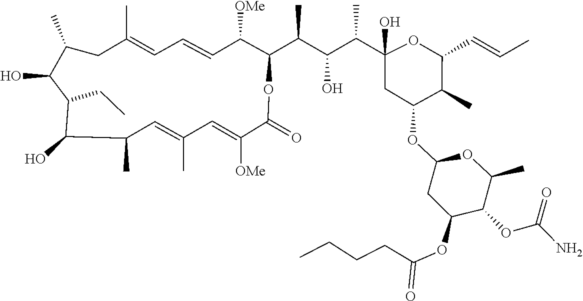 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](OC(=O)CCCC)[C@@H](OC(N)=O)[C@H](C)O2)[C@@H]1C] |
| 35 | 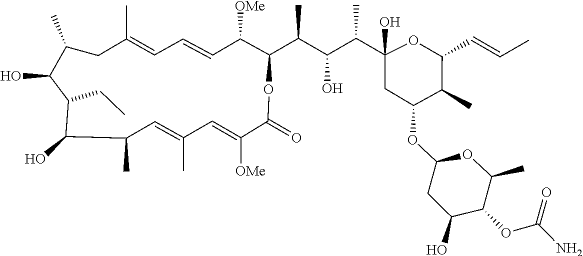 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](OC(N)=O)[C@H](C)O2)[C@@H]1C] |
| 36 | 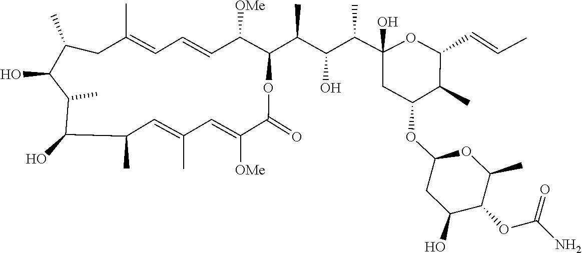 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](O)[C@@H](OC(N)=O)[C@H](C)O2)[C@@H]1C] |
| 37 | 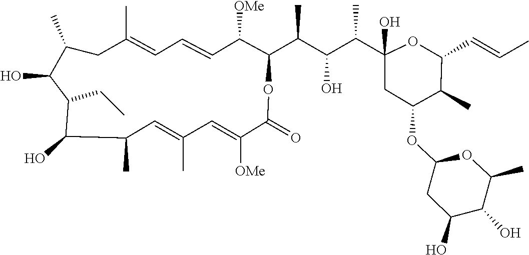 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C] |
| 38 | 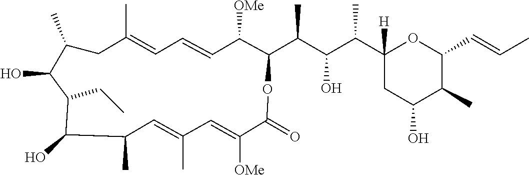 [SMILES: C/C=C/[C@H]1O[C@@H]([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O)[C@@H]1C] |
| 39 | 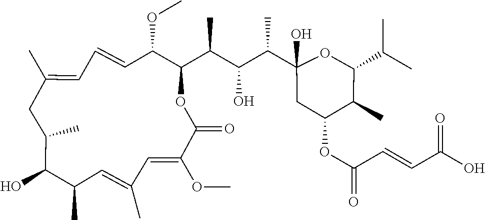 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)/C=C/C(=O)O)[C@H](C)[C@@H](C(C)C)O2)OC1=O] |
| 40 | 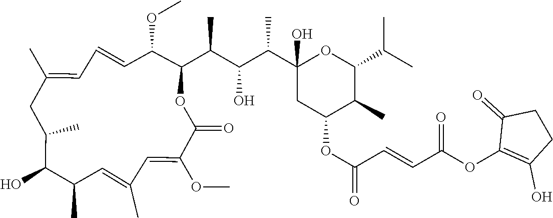 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)/C=C/C(=O)OC3=C(O)CCC3=O)[C@H](C)[C@@H](C(C)C)O2)OC1=O] |
| 41 | 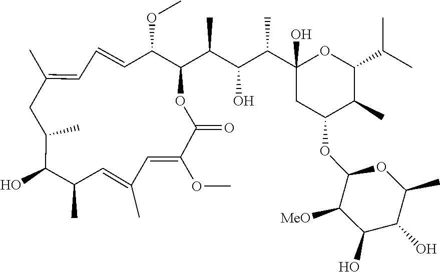 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O[C@H]3O[C@@H](C)[C@H](O)[C@@H](O)[C@H]3OC)[C@H](C)[C@@H](C(C)C)O2)OC1=O] |
| 42 | 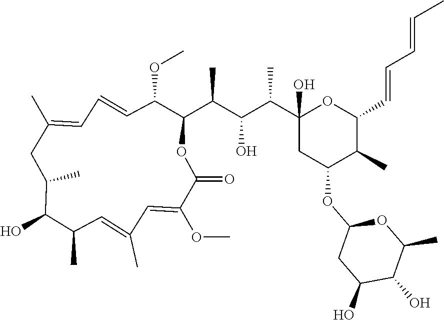 [SMILES: C/C=C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)C[C@@H](O[C@@H]2C[C@H](O)[C@@H](O)[C@H](C)O2)[C@@H]1C] |
| 43 | 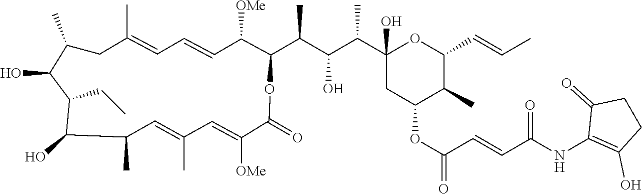 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](OC(=O)/C=C/C(=O)NC2=C(O)CCC2=O)[C@@H]1C] |

As used herein, the term “alkyl” refers to straight chained and branched saturated hydrocarbon groups containing one to twenty-two carbon atoms, for example, one to twenty carbon atoms, or one to ten carbon atoms, or one to four carbon atoms. The term Cₙ means the alkyl group has “n” carbon atoms. For example, C₄ alkyl refers to an alkyl group that has 4 carbon atoms. C₁₋₂₀ alkyl and C₁-C₂₀ alkyl refer to an alkyl group having a number of carbon atoms encompassing the entire range (i.e., 1 to 20 carbon atoms), as well as all subgroups (e.g., 1-18, 2-15, 1-5, 3-10, 1, 2, 3, 4, 5, 6, 7, 8, 9, 10, 11, 12, 13, 14, 15, 16, 17, 18, 19, and 20 carbon atoms). Nonlimiting examples of alkyl groups include methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkenyl” as used herein means a straight or branched chain hydrocarbon comprising one or more double bonds.

As used herein, the term “cycloalkyl” refers to an aliphatic cyclic hydrocarbon group containing five to eight carbon atoms (e.g., 5, 6, 7, or 8 carbon atoms). The term Cₙ means the cycloalkyl group has “n” carbon atoms. For example, C₅ cycloalkyl refers to a cycloalkyl group that has 5 carbon atoms in the ring. C₅₋₈ cycloalkyl and C₅-C₈ cycloalkyl refer to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group.

As used herein, the term “aryl” refers to monocyclic or polycyclic (e.g., fused bicyclic and fused tricyclic) carbocyclic aromatic ring systems, having 6 to 22 ring carbon atoms. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “heterocycle” refers to either a heteroaryl or heterocycloalkyl.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of two to eight atoms, of which 1, 2, or 3 of those atoms are heteroatoms independently selected from the group consisting of oxygen, nitrogen, and sulfur, and the remaining atoms in the ring are carbon atoms. Nonlimiting examples of heterocycloalkyl groups include piperidine, tetrahydrofuran, tetrahydropyran, dihydrofuran, morpholine, and the like. Heterocycloalkyl groups can be saturated or partially unsaturated ring systems optionally substituted with, for example, one to three groups, independently selected from alkyl, alkenyl, OH, C(O)NH₂, NH₂, oxo (═O), aryl, haloalkyl, halo, and OH. Heterocycloalkyl groups optionally can be further N-substituted with alkyl, hydroxyalkyl, alkylene-aryl, and alkylene-heteroaryl. The heterocycloalkyl groups described herein can be isolated or fused to another heterocycloalkyl group, a cycloalkyl group, an aryl group, and/or a heteroaryl group. When a heterocycloalkyl group is fused to another heterocycloalkyl group, then each of the heterocycloalkyl groups can contain three to eight total ring atoms, and one to three heteroatoms. In some embodiments, the heterocycloalkyl groups described herein comprise one oxygen ring atom (e.g., oxiranyl, oxetanyl, tetrahydrofuranyl, and tetrahydropyranyl).

As used herein, the term “heteroaryl” refers to a cyclic aromatic ring having five to twelve total ring atoms (e.g., a monocyclic aromatic ring with 5-6 total ring atoms), and containing one to three heteroatoms selected from nitrogen, oxygen, and sulfur in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Heteroaryl groups can be isolated (e.g., pyridyl) or fused to another heteroaryl group (e.g., purinyl), a cycloalkyl group (e.g., tetrahydroquinolinyl), a heterocycloalkyl group (e.g., dihydronaphthyridinyl), and/or an aryl group (e.g., benzothiazolyl and quinolyl). Examples of heteroaryl groups include, but are not limited to, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

In some cases, R¹⁰ is C(O)R⁶. In embodiments, R⁶ is C₁₋₆ alkyl. In embodiments, R⁶ is a sugar moiety. A sugar can be a pentose, hexose, heptose, or an amino sugar (e.g., aminopentose, aminohexose, aminoheptose, or a neuraminic acid), for example. Some contemplated sugars include, but are not limited to, allose, altrose, glucose, mannose, gulose, idose, galactose, talose, sialic acid, glucosamine, galactosamine, fructose, arabinose, dextrose, sorbose, psicose, tagatose, sucrose, lactose, maltose, trehalose, cellobiose, chitobiose, lactulose, kojibiose, nigerose, isomaltose, sophorose, laminaribose, gentiobiose, turanose, matlulose, plaltinose, gentiobiulose, mannobiose, melibiose, melibiulose, rutinose, rutinulose, N-acetylglucosamine, fucose, N-acetylneuraminic acid, sialic acid, xylobiose, ribose, rhamnose, xylose, cladinose, mycinose, javose, 2-deoxy-β-D-rhamnose, and the like. Contemplated amino sugars include desosamine, mycaminose, and the like. For the avoidance of doubt, the terms “carbohydrate,” “sugar,” and “saccharide” are all used interchangeably.

In some cases, R¹⁰ is a sugar moiety. In some cases, R¹⁰ is an amino sugar moiety.

In some embodiments, R¹⁰ is C(O)NHR⁹, wherein R⁹ is C₁₋₂₀ alkyl, C₂₋₁₂ alkenyl, C₅-C₈ cycloalkyl, C₂₋₁₂ heterocycloalkyl, or Ar¹. In some cases, R⁹ is C₁₋₂₀ alkyl. In some cases, R⁹ is C₁₋₆ alkyl. In some cases, R⁹ is C₂₋₁₂ alkenyl. In some cases, R⁹ is C₅-C₈ cycloalkyl. In some cases, R⁹ is Ar¹. In some cases, R⁹ is Ph. In some cases, R⁹ is C₂₋₁₂ heterocycloalkyl.

As described herein, compounds described herein may optionally be substituted with one or more substituents, such as illustrated generally below, or as exemplified by particular classes, subclasses, and species described herein. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. In some cases, the substituent is selected from group A: halo, CN, OH, CO₂H, CHO, NH₂, oxo, NO₂, C₁₋₆alkyl, C₁₋₆haloalkyl, C₁₋₆alkoxy, C₁₋₆alkylthio, C₁₋₆ alkyl-OH, C₃₋₁₀carbocyclyl, 3-7 membered heterocyclyl, C₃₋₁₀carbocyclyl-C₁₋₆alkoxy, C₃₋₁₀carbocyclyl-O—C₁₋₆alkylene, C₃₋₁₀carbocyclyl-C₁₋₆alkoxy-C₁₋₆alkylene, 3-7 membered heterocyclyl-C₁₋₆alkoxy, 3-7 membered heterocyclyl-O—C₁₋₆alkylene, 3-7 membered heterocyclyl-C₁₋₆alkoxy-C₁₋₆alkylene, C₁₋₆haloalkoxy, C₁₋₆alkoxy-C₁₋₆alkylene, C₁₋₆alkoxy-C₁₋₆alkoxy, C₁₋₆alkyl-C(O)—, C₁₋₆alkyl-C(O)O—, NHC₁₋₆alkyl, C₁₋₆alkyl-C(O)NH—, C₁₋₆haloalkyl-C(O)NH, C₁₋₆alkyl-NHC(O)—, C₁₋₆alkyl-SO₂—, C₁₋₆alkyl-SO—, and C₁₋₆alkylSO₂NH—.

Selection of substituents and combinations of substituents contemplated herein are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is drawn specifically.

Therefore, single stereochemical isomers as well as enantiomeric, diastereomeric, cis/trans, conformational, and rotational mixtures of the present compounds are within the scope of the disclosure.

Unless otherwise indicated, all tautomeric forms of the compounds described herein are within the scope of the disclosure.

Additionally, unless otherwise indicated, structures depicted herein are also meant to include compounds that differ only in the presence of one or more isotopically enriched atoms. Discussion of an element is intended to include all isotopes of that element. For example, a substituent shown as a hydrogen includes where that hydrogen is in the deuterium or tritium isotope form, and a carbon atom can be present as a ¹³C— or ¹⁴C— carbon isotope.

It is understood that selections of values of each variable are those that result in the formation of stable or chemically feasible compounds.

### Generation of O-acyl Baf A1 and CMA Derivatives

The analogs herein can have modification of free hydroxyl-groups of the target plecomacrolides. These modifications can be further useful in probing the tolerance of alterations at specific positions on each precursor, helping guide future derivatization, and localizing the pharmacophore of these compounds. This is accomplished via treatment of CMA and Baf A1, or other plecomacrolides, with commercially available anhydrides, yielding C7 and/or C21 (Baf A1), C9 and/or C23 (CMA) O-acyl derivatives (Deeg et al., 1987; Dröse et al., 2001; Ingenhorst et al., 2001). These analogs are tested for inhibition of Nef-mediated MHC-I downmodulation and toxicity as shown in FIG. 4.

### Regioselective Glycodiversification

Glycosylation of small molecules can significantly improve compound solubility, activity, and reduce off target toxicity (Elshahawi et al., 2015). Altering sugar moieties has been shown to be an effective method of diversifying chemical structure and optimizing bioactivity. As such, high interest has been placed on modifying drug-leads via addition or substitution of alternative sugar groups (Fu et al., 2003; Tay et al., 2017). Regiospecific glycosylation of macrolactones which involves harvesting rare sugars from commercial natural products (e.g. tylosin, erythromycin, and spiramycin) activating them chemically as thioglycoside or glycosyl fluorides for addition of the sugar donor to a target hydroxyl group (Anzai et al., 2008), can be used in the preparing the analogs herein.

The plecomacrolides are a group of macrolactone compounds where glycodiversification can be useful in tailoring the properties of the analogs. CMA (FIG. 2), differs from Baf A1 in that it is glycosylated at the C23-position with 2-deoxy-β-D-rhamnose as R3 (FIG. 7). This difference is analyzed to determine whether it is functionally important for CMA potency. Analogs herein include 2-deoxy-β-D-rhamnose added to the corresponding C21 OH group of Baf A1. Bioactivity assays such as those shown in FIG. 4, determine whether this alteration is sufficient to shift the Baf A1 curve to the left towards the CMA curve. In addition, this semi-synthetic approach can be used towards the regioselective introduction of a variety of alternative chemically activated donor sugars (e.g. cladinose, mycinose, javose, desosamine, mycaminose, etc.).

### Succinimidyl (NHS) Ester Activation Strategy for Plecomacrolide Diversification

The Baf A1 and CMA can be selectively “activated” using a coupling reaction with succinimidyl (NHS) esters (Morpurgo et al., 1999). This enables a site-selective high-throughput derivatization methodology. In embodiments, a 96-well plate format can be used containing a library of commercially available primary alkyl, alkenyl, cyclic, aromatic, and hetero-aromatic amines (FIG. 7). These groups are coupled with the NHS-Baf A1/CMA precursors directly in the plates. The samples are dried and submitted for high-throughput testing without further purification required. This approach enables screening of hundreds of analogs. The top performing derivatives that display improved activity, or lower toxicity are synthesized in large scale (>100 mg) starting with the Baf A1 and/or CMA precursor for further preclinical testing.

In addition, the strains utilized herein are engineered Streptomyces with core natural product biosynthetic genes disrupted (Jung et al., 2008; Jung et al., 2006). Genes encoding tailoring enzymes, including those involved in sugar biosynthesis are maintained and highly expressed. Incubation of Baf A1 and CMA aglycone with these recombinant strains generates chemoenzymatic modifications specific to the engineered systems. CMA naturally contains a 4-carbamyl-2-deoxy-β-D-rhamnose sugar at position R3 (FIG. 7). Based on a similar strategy employed previously (Jung et al., 2008; Jung et al., 2006), a biotransformation strain is engineered to be capable of adding this moiety to Baf A1 and other derivatives. CMA producing strains are modified via deletion of the core polyketide synthase genes responsible for scaffold production. Gene deletion is accomplished using traditional methods, or CRISPR/Cas9-based genome editing developed for Streptomyces (Alberti and Corre, 2019). New analogs are purified and tested for anti-Nef activity, V-ATPase inhibition and cellular toxicity.

### Methods of Use

The analogs described herein or pharmaceutically acceptable salts thereof can be used to modulate an HIV Nef. Modulating an HIV Nef includes inhibiting an HIV Nef.

The term “HIV Nef-associated disorder” is used herein to mean diseases or disorders whose status or progression is influenced by the expression of HIV Nef in a patient. A non-limiting example of an HIV Nef-associated disorder is HIV infection, e.g., HIV-1 infection.

As used herein, “HIV” refers to the human immunodeficiency virus. HIV includes, without limitation, HIV-1. HIV-1 includes but is not limited to extracellular virus particles and the forms of HIV-1 associated with HIV-1 infected cells. The human immunodeficiency virus (HIV) may be either of the two known types of HIV (HIV-1 or HIV-2). As used herein, HIV-1 refers to any of the known major subtypes (classes A, B, C, D, E, F, G, H, or J), outlying subtype (Group O), yet to be determined subtypes of HIV-1, and recombinations thereof.

As used herein, “HIV infection” refers to infection of a subject with HIV.

The terms, “disease”, “disorder”, and “condition” may be used interchangeably herein to refer to an HIV Nef-associated medical or pathological condition, such as HIV infection.

As used herein, the terms “subject”, “host”, and “patient” are used interchangeably. The terms “subject”, “host”, and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, or mouse) and a primate (e.g., a monkey, chimpanzee, or human), and more specifically a human. In some embodiments, the subject is a non-human animal such as a farm animal (e.g., a horse, cow, pig or sheep), or a pet (e.g., a dog, cat, guinea pig or rabbit). In a preferred embodiment, the subject is a “human”.

As used herein, the terms “treat”, “treatment,” and “treating” refer to therapeutic treatment and/or prophylactic treatments. For example, therapeutic treatments include the reduction or amelioration of the progression, severity and/or duration of HIV infection, or the amelioration of one or more symptoms (specifically, one or more discernible symptoms) of HIV infection, resulting from the administration of one or more therapies (e.g., one or more therapeutic agents such as a compound or composition described herein). In specific embodiments, the therapeutic treatment includes the amelioration of at least one measurable physical parameter of an HIV infection. In other embodiments, the therapeutic treatment includes the inhibition of the progression of an HIV infection, either physically by, e.g., stabilization of a discernible symptom, physiologically by, e.g., stabilization of a physical parameter, or both. In other embodiments, the therapeutic treatment includes the reduction or stabilization of HIV infections. Antiviral drugs can be used in the community setting to treat people who already have HIV infection to reduce the severity of symptoms and suppress the infection. Treating and HIV infection includes reducing or eliminating an HIV reservoir in a patient.

As used herein, the term “HIV reservoir” refers to a group of cells in a patient that are infected with HIV but have not produced new HIV (i.e., are in a latent stage of infection) for many months or years. Very early during acute HIV infection, a latent reservoir is established and despite effective combination anti-retroviral therapy (cART), HIV persists in latently infected cells. If a patient having a latent HIV infection stops treatment with cART, the presence of an HIV reservoir in a patient can allow an active HIV infection to become re-established in the patient.

The terms “prophylaxis”, “prophylactic”, “prophylactic use”, and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease.

As used herein, prophylactic use includes use to prevent contagion or spread of the infection in populations or individuals at high risk of HIV infection. Prophylactic use may also include treating a person who is not ill with HIV or not considered at high risk for contracting HIV, in order to reduce the chances of becoming infected with HIV and passing it on to another pereson.

In some embodiments, the methods of the disclosure are applied as a prophylactic measure to members of a community or population group, specifically humans, in order to prevent the spread of infection.

As used herein, an “effective amount” refers to an amount sufficient to elicit the desired biological response. In the present disclosure the desired biological response is to inhibit the replication of HIV, to reduce the amount of HIV, or to reduce or ameliorate the severity, duration, progression, or onset of an HIV infection, prevent the advancement of an HIV infection, prevent the recurrence, development, onset or progression of a symptom associated with an HIV infection, or enhance or improve the prophylactic or therapeutic effect(s) of another therapy used against HIV infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other antiviral agents, e.g., when co-administered with an anti-HIV medication, an effective amount of the second agent will depend on the type of drug used. A safe amount is one with minimal side effects, as can readily be determined by those skilled in the art. Suitable dosages are known for approved agents and can be adjusted by the skilled artisan according to the condition of the subject, the type of condition(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

As used herein, a “safe and effective amount” of a compound or composition described herein is an effective amount of the compound or composition which does not cause excessive or deleterious side effects in a patient.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe a safe and effective amount of the compounds described herein required to treat, to prevent, inhibit (fully or partially) or arrest the progress of the disease.

Dosages of the compounds described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc.

### Pharmaceutically Acceptable Salts

The analogs described herein can exist in free form, or, where appropriate, as salts. Those salts that are pharmaceutically acceptable are of particular interest since they are useful in administering the analogs described below for medical purposes. Salts that are not pharmaceutically acceptable are useful in manufacturing processes, for isolation and purification purposes, and in some instances, for use in separating stereoisomeric forms of the compounds described herein or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the analogs described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxylic acid group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this disclosure includes mixtures/combinations of different pharmaceutically acceptable salts and also mixtures/combinations of compounds in free form and pharmaceutically acceptable salts.

### Pharmaceutical Compositions

The analogs described herein can be formulated into pharmaceutical compositions that further comprise a pharmaceutically acceptable carrier, diluent, adjuvant or vehicle. In some embodiments, the present disclosure relates to a pharmaceutical composition comprising an analog described herein, and a pharmaceutically acceptable carrier, diluent, adjuvant or vehicle. In some embodiments, the present disclosure includes a pharmaceutical composition comprising a safe and effective amount of a compound described herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating an HIV infection in a patient.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Administration Methods

The analogs and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, to the pulmonary system, such as by using an inhaler, such as a metered dose inhaler (MDI), or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an analog described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions described herein may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as polysorbates, sorbitan esters, and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds described herein include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The compounds for use in the methods described herein can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The disclosure will be more fully understood by reference to the examples described herein which detail exemplary embodiments. These examples should not, however, be construed as limiting the scope of the disclosure. All citations throughout the disclosure are hereby expressly incorporated by reference.

### EXAMPLES

### Example 1: Bafilomycin A₂ (Compound 1)

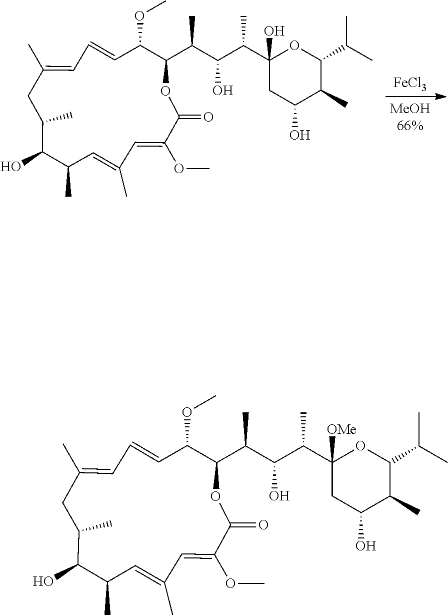

Bafilomycin A₁ (74 mg, 0.090 mmol) was dissolved in MeOH (2 mL) under anhydrous conditions and cooled to 0° C. FeCl₃ (2.9 mg, 0.018 mmol, 0.2 eq) was added to the solution. The FeCl₃ was added to the reaction after first dissolving it in a separate flask with MeOH. The reaction was ran at RT for 15-30 minutes, checking TLC (55% ethyl acetate/hexane) for consumption of starting material. The reaction was quenched with phosphate buffer (1.0 M, 7.1 pH). The reaction was dissolved in DCM, washed with water, brine, dried over MgSO₄, filtered, and concentrated. The crude reaction was then purified by flash column chromatography (30-60% ethyl acetate/hexane) to yield the desired compound. Bafilomycin A₂ (Compound 1) ¹H NMR (599 MHz, Acetone, 298 K) δ 6.69 (d, J=0.9 Hz, 1H), 6.66 (dd, J=15.0, 10.8 Hz, 1H), 5.96 (dt, J=8.9, 1.2 Hz, 1H), 5.81 (d, J=10.8 Hz, 1H), 5.17 (dd, J=15.0, 9.0 Hz, 1H), 5.09 (dd, J=8.1, 1.5 Hz, 1H), 4.05 (d, J=8.5 Hz, 1H), 4.04 (d, J=5.3 Hz, 1H), 3.93-3.89 (m, 1H), 3.66 (s, 3H), 3.52-3.49 (m, 1H), 3.46 (q, J=4.1, 3.1 Hz, 1H), 3.32 (dp, J=8.2, 3.0, 2.5 Hz, 1H), 3.23 (s, 3H), 3.11-3.05 (m, 1H), 3.04 (s, 3H), 2.78 (s, 1H), 2.59-2.46 (m, 1H), 2.19 (dd, J=13.3, 4.3 Hz, 1H), 2.12-2.06 (m, 1H), 2.05 (p, J=2.2 Hz, 2H), 2.04-1.99 (m, 1H), 1.98 (d, J=1.2 Hz, 3H), 1.97-1.93 (m, 1H), 1.92 (d, J=1.4 Hz, 3H), 1.88 (p, J=7.1 Hz, 1H), 1.48 (dd, J=13.4, 10.6 Hz, 1H), 1.27 (dd, J=10.0, 6.6 Hz, 1H), 1.26-1.19 (m, 1H), 1.08 (dd, J=14.1, 7.2 Hz, 1H), 1.05 (d, J=7.0 Hz, 3H), 1.03 (d, J=6.9 Hz, 3H), 0.97 (d, J=7.0 Hz, 3H), 0.95 (d, J=6.9 Hz, 3H), 0.92 (t, J=7.0 Hz, 6H), 0.89 (d, J=6.8 Hz, 3H). ¹³C NMR (151 MHz, Acetone) δ 167.23, 145.31, 144.50, 142.13, 134.06, 133.61, 132.68, 127.27, 125.32, 103.84, 83.84, 80.45, 78.18, 77.65, 70.39, 70.34, 60.19, 55.72, 46.68, 42.32, 41.52, 41.24, 40.06, 39.84, 39.17, 38.05, 29.14, 22.40, 21.12, 20.21, 17.80, 14.63, 14.09, 12.63, 11.11, 7.90.

### Example 2: 19-deoxy Bafilomycin (Compound 2)

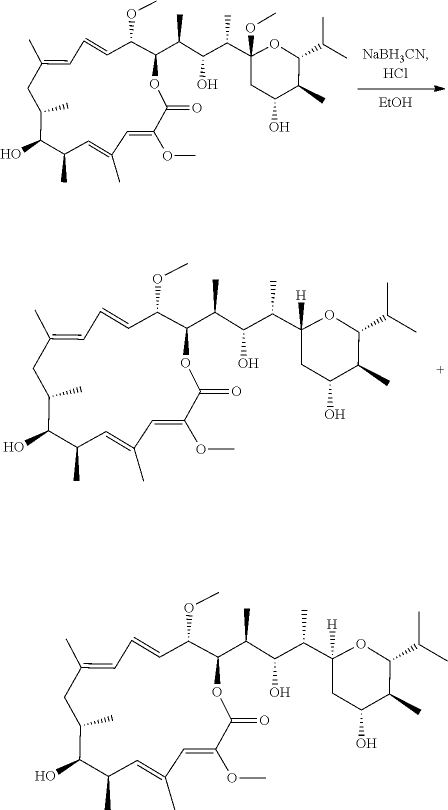 [SMILES: "EtOH" ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(OC)C[C@@H](O)[C@H](C)[C@@H](C(C)C)O2)OC1=O ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](O)[C@H](C)[C@@H](C(C)C)O2)OC1=O ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@H]2C[C@@H](O)[C@H](C)[C@@H](C(C)C)O2)OC1=O]

Bafilomycin A₂ was dissolved in EtOH and stirred under nitrogen. NaBH₃CN and HCl were added to the reaction flask that was then left to run for approximately 1 hour, checking by TLC to determine the reaction's completeness. The crude reaction mixture was extracted with DCM (×4). The organics were combined, washed with water, brine, dried over NaSO₄, filtered and concentrated. The crude material was purified further by flash column chromatography (12-100% ethyl acetate/hexane). 19-deoxy Bafilomycin (Compound 2) ¹H NMR (599 MHz, Acetone) δ 6.68 (s, 1H), 6.64 (dd, J=15.0, 10.8 Hz, 1H), 5.94 (d, J=8.9 Hz, 1H), 5.80 (d, J=10.8 Hz, 1H), 5.22-5.18 (m, 1H), 5.17 (dd, J=14.6, 8.4 Hz, 1H), 4.04 (t, J=8.3 Hz, 1H), 4.00 (d, J=5.4 Hz, 1H), 3.81-3.76 (m, 1H), 3.64 (t, J=4.1 Hz, 1H), 3.62 (s, 3H), 3.37 (ddd, J=11.5, 8.2, 1.8 Hz, 1H), 3.32 (s, 1H), 3.27 (td, J=10.2, 4.6 Hz, 1H), 3.23 (s, 3H), 2.79 (dd, J=10.0, 2.2 Hz, 1H), 2.60 (s, 1H), 2.54 (ddd, J=9.0, 7.0, 2.0 Hz, 1H), 2.14 (s, 1H), 2.07-2.00 (m, 3H), 1.97 (d, J=1.3 Hz, 3H), 1.90 (s, 3H), 1.89-1.82 (m, 2H), 1.69-1.61 (m, 1H), 1.26 (s, 1H), 1.15 (q, J=11.5 Hz, 1H), 1.05 (d, J=7.1 Hz, 3H), 0.95 (d, J=6.8 Hz, 3H), 0.93 (d, J=6.9 Hz, 3H), 0.91 (d, J=6.5 Hz, 3H), 0.87 (d, J=6.9 Hz, 3H), 0.84 (d, J=7.0 Hz, 3H), 0.79 (d, J=6.8 Hz, 3H). ¹³C NMR (151 MHz, CDCl₃) δ 166.63, 144.72, 144.14, 142.34, 133.80, 133.15, 132.64, 127.27, 125.35, 85.14, 84.03, 80.50, 77.68, 77.62, 74.22, 70.45, 59.97, 55.70, 42.31, 42.06, 41.26, 40.83, 40.71, 38.76, 38.12, 29.23, 22.52, 21.58, 20.06, 17.91, 14.83, 14.19, 12.67, 10.64, 8.89.

### Example 3: 21-Acetyl Bafilomycin (Compound 3)

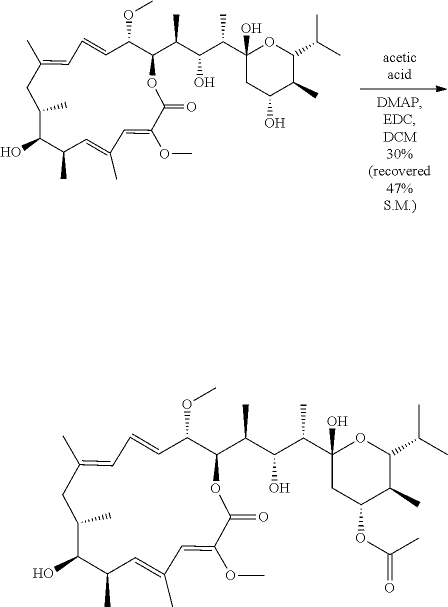

DMAP (6.47 mg, 53.0 μmol, 2.1 eq) and EDC (13.9 mg, 55.5 μmol, 2.2 eq) were added to a solution of bafilomycin A₁ (15.0 mg, 25.2 μmol) in DCM (2.92 mL) at RT under anhydrous conditions. Acetic acid (1.0 eq, approximately 0.25 mL of a 1.44 μg/mL solution) was added in increments of 0.25 eq every 30 minutes to prevent double acetylation. The reaction was ran an addition hour after the last addition of acetic acid. The solution was concentrated before direct purification by pTLC (20% acetone in hexanes) to yield 21-acetyl bafilomycin. 21-Acetyl Bafilomycin (Compound 3) ¹H NMR (599 MHz, Acetone) δ 6.71 (d, J=0.8 Hz, 1H), 6.68 (dd, J=15.0, 10.8 Hz, 1H), 5.96 (dt, J=8.9, 1.2 Hz, 1H), 5.80 (d, J=10.9 Hz, 1H), 5.38 (d, J=2.1 Hz, 1H), 5.14 (dd, J=15.0, 9.2 Hz, 1H), 4.97 (dd, J=8.4, 1.4 Hz, 1H), 4.91 (td, J=10.9, 4.8 Hz, 1H), 4.76 (dd, J=4.4, 1.1 Hz, 1H), 4.18 (ddd, J=10.7, 4.4, 1.9 Hz, 1H), 4.09-4.03 (m, 2H), 3.64 (s, 3H), 3.58 (dd, J=10.4, 2.2 Hz, 1H), 3.31 (td, J=6.5, 5.7, 2.0 Hz, 1H), 3.24 (s, 3H), 2.55 (tt, J=9.2, 6.5 Hz, 1H), 2.25 (dd, J=11.8, 4.9 Hz, 1H), 2.20-2.12 (m, 1H), 2.08-1.99 (m, 2H), 2.00 (s, 3H), 1.98 (d, J=1.2 Hz, 3H), 1.97-1.84 (m, 5H), 1.83 (dt, J=8.8, 6.4 Hz, 1H), 1.52 (tdd, J=13.0, 8.5, 5.2 Hz, 1H), 1.21 (td, J=11.5, 2.1 Hz, 1H), 1.05 (d, J=7.0 Hz, 3H), 0.99 (d, J=7.2 Hz, 3H), 0.94 (d, J=6.8 Hz, 3H), 0.92 (d, J=6.8 Hz, 3H), 0.87 (d, J=6.9 Hz, 3H), 0.81 (d, J=3.7 Hz, 3H), 0.80 (d, J=4.1 Hz, 3H). ¹³C NMR (151 MHz, Acetone) δ 170.62, 167.80, 145.72, 144.83, 141.95, 134.45, 134.20, 132.78, 127.02, 125.23, 99.71, 83.36, 80.41, 77.41, 76.57, 74.14, 71.57, 60.17, 55.67, 42.96, 42.27, 41.70, 40.78, 38.99, 38.29, 38.01, 28.74, 22.25, 21.62, 21.07, 20.35, 17.73, 14.59, 14.15, 12.53, 10.30, 7.36.

### Example 4: 21-pentanone Bafilomycin (Compound 4)

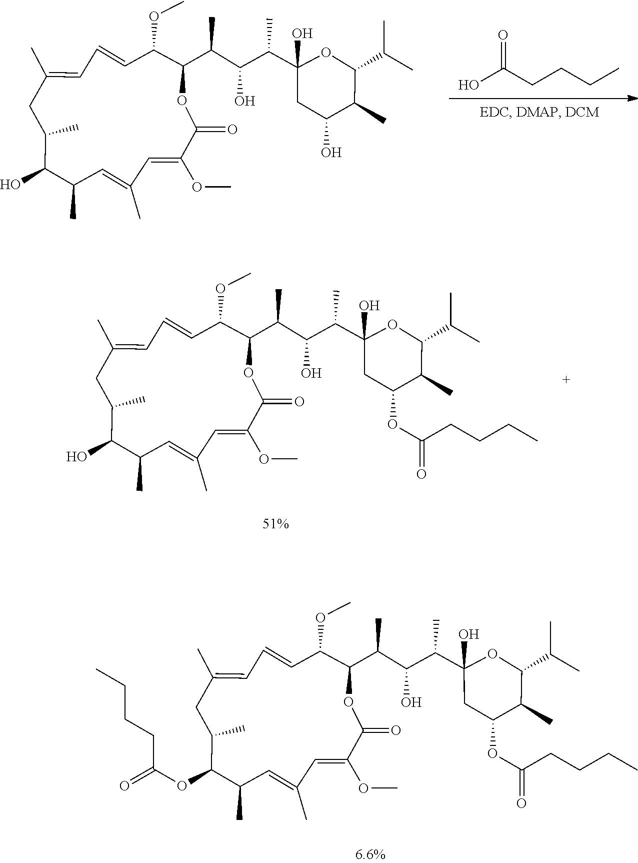

Pentanoic acid (3.94 mg, 38.5 μmol, 2 eq) was added to a solution of bafilomycin A₁ (12.0 mg, 19.3 μmol) in DCM (2.36 mL) under anhydrous conditions. DMAP (4.94 mg, 40.5 μmol, 2.1 eq) and EDC (8.13 mg, 42.4 μmol, 2.2 eq) were added to the solution and stirred overnight. The reaction mixture was concentrated and purified by pTLC (20% acetone in hexanes) to yield 21-pentanoate Bafilomycin and 7,21-dipentanoate Bafilomycin. 21-pentanone Bafilomycin (Compound 4) ¹H NMR (599 MHz, Acetone) δ 6.71 (s, 1H), 6.68 (dd, J=15.0, 10.8 Hz, 1H), 5.96 (d, J=8.8 Hz, 1H), 5.80 (d, J=10.8 Hz, 1H), 5.38 (d, J=2.1 Hz, 1H), 5.15 (dd, J=15.0, 9.2 Hz, 1H), 4.97 (dd, J=8.4, 1.4 Hz, 1H), 4.93 (td, J=10.8, 4.8 Hz, 1H), 4.76 (dd, J=4.3, 1.0 Hz, 1H), 4.18 (ddd, J=10.7, 4.3, 1.8 Hz, 1H), 4.09-4.03 (m, 2H), 3.64 (s, 3H), 3.59 (dd, J=10.4, 2.2 Hz, 1H), 3.31 (ddd, J=7.2, 5.5, 2.0 Hz, 1H), 3.24 (s, 3H), 2.56 (pd, J=7.0, 3.5 Hz, 1H), 2.30 (td, J=7.5, 4.0 Hz, 2H), 2.25 (dd, J=11.7, 4.8 Hz, 1H), 2.21-2.12 (m, 1H), 2.09-1.98 (m, 2H), 1.98 (d, J=1.2 Hz, 3H), 1.93 (s, 3H), 1.89 (dtd, J=18.0, 7.0, 3.1 Hz, 2H), 1.83 (dd, J=8.1, 6.3 Hz, 1H), 1.63-1.55 (m, 2H), 1.58-1.49 (m, 1H), 1.35 (h, J=7.4 Hz, 2H), 1.21 (td, J=11.5, 2.1 Hz, 1H), 1.05 (d, J=7.0 Hz, 3H), 0.99 (d, J=7.1 Hz, 3H), 0.95 (d, J=6.9 Hz, 3H), 0.92 (d, J=5.0 Hz, 3H), 0.91 (t, J=6.4 Hz, 3H), 0.88 (d, J=6.8 Hz, 3H), 0.81 (d, J=3.7 Hz, 3H), 0.80 (d, J=3.9 Hz, 3H). ¹³C NMR (151 MHz, Acetone) δ 173.28, 167.82, 145.71, 144.82, 141.95, 134.45, 134.20, 132.79, 127.04, 125.24, 99.72, 83.34, 80.42, 77.42, 76.57, 73.97, 71.58, 60.17, 55.67, 42.95, 42.27, 41.70, 40.82, 39.00, 38.28, 38.01, 34.66, 28.75, 27.90, 22.88, 22.25, 21.63, 20.36, 17.73, 14.60, 14.16, 14.01, 12.58, 10.30, 7.38.

### Example 5: 21-nonanoate Bafilomycin (Compound 5)

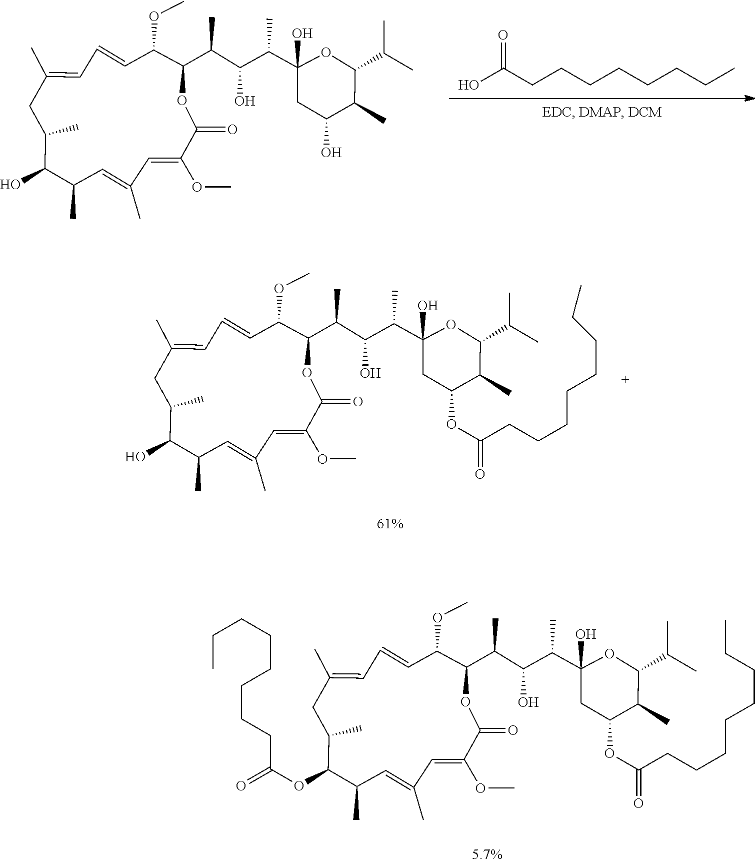

Nonanoic acid (6.10 mg, 38.5 μmol, 2 eq) was added to a solution of bafilomycin A₁ (12.0 mg, 19.3 μmol) in DCM (2.36 mL) under anhydrous conditions. DMAP (4.94 mg, 40.5 μmol, 2.1 eq) and EDC (8.13 mg, 42.4 μmol, 2.2 eq) were added to the solution and stirred overnight. The reaction mixture was concentrated and purified by pTLC (20% acetone in hexanes) to yield 21-nonanoate Bafilomycin and 7,21-dinonanoate bafilomycin. 21-nonanoate Bafilomycin (Compound 5) ¹H NMR (599 MHz, CDCl₃) δ 6.67 (s, 1H), 6.50 (dd, J=15.0, 10.6 Hz, 1H), 5.81 (d, J=10.6 Hz, 1H), 5.77 (d, J=9.1 Hz, 1H), 5.47 (d, J=2.0 Hz, 1H), 5.16 (dd, J=15.0, 9.3 Hz, 1H), 5.01-4.93 (m, 2H), 4.61 (d, J=4.1 Hz, 1H), 4.15-4.09 (m, 1H), 3.88 (t, J=9.0 Hz, 1H), 3.64 (s, 3H), 3.60 (dd, J=10.4, 2.2 Hz, 1H), 3.30 (dd, J=7.1, 3.9 Hz, 1H), 3.24 (s, 3H), 2.58-2.50 (m, 1H), 2.32 (dd, J=11.8, 4.8 Hz, 1H), 2.28 (t, J=7.5 Hz, 2H), 2.13 (td, J=13.6, 12.3, 5.3 Hz, 2H), 1.99 (s, 3H), 1.94 (s, 4H), 1.93-1.84 (m, 2H), 1.75 (q, J=7.2 Hz, 1H), 1.62 (q, J=7.2, 5.2 Hz, 2H), 1.54 (q, J=4.1 Hz, 1H), 1.34-1.23 (m, 12H), 1.17 (td, J=11.4, 2.1 Hz, 1H), 1.10 (s, 0H), 1.07 (d, J=7.0 Hz, 3H), 1.02 (d, J=7.1 Hz, 3H), 0.94 (d, J=6.4 Hz, 3H), 0.91 (d, J=6.8 Hz, 2H), 0.88 (t, J=6.9 Hz, 3H), 0.82 (t, J=6.5 Hz, 6H), 0.77 (d, J=6.7 Hz, 3H). ¹³C NMR (151 MHz, CDCl₃) δ 173.18, 167.28, 142.98, 142.70, 141.29, 133.48, 133.02, 132.98, 127.26, 125.31, 98.81, 82.23, 81.22, 76.85, 75.58, 73.60, 70.61, 59.94, 55.52, 42.06, 41.21, 40.20, 40.01, 38.21, 37.17, 36.69, 34.74, 31.81, 29.25-29.13 (m), 27.92, 25.16, 22.65, 21.66, 21.10, 20.17, 17.28, 14.29, 14.07 (d, J=9.3 Hz), 12.30, 9.84, 7.08.

### Example 6: 21-benzyl Bafilomycin (Compound 6)

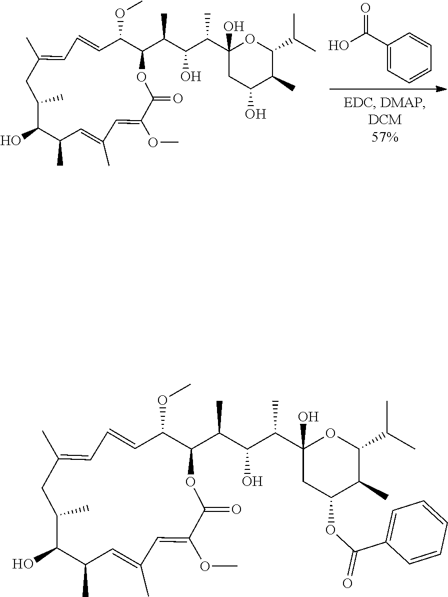

Benzoic acid (4.71 mg, 38.5 μmol, 2 eq) was added to a solution of bafilomycin A₁ (12.0 mg, 19.3 μmol) in DCM (2.36 mL) under anhydrous conditions. DMAP (4.94 mg, 40.5 μmol, 2.1 eq) and EDC (8.13 mg, 42.4 μmol, 2.2 eq) were added to the solution and stirred overnight. The reaction mixture was concentrated and purified by pTLC (20% acetone in hexanes) to yield the desired product. 21-benzyl Bafilomycin (Compound 6) ¹H NMR (599 MHz, Acetone) δ 8.07-8.02 (m, 2H), 7.66-7.61 (m, 1H), 7.56-7.49 (m, 2H), 6.72 (d, J=0.9 Hz, 1H), 6.68 (dd, J=15.0, 10.8 Hz, 1H), 5.97 (dt, J=8.9, 1.2 Hz, 1H), 5.81 (d, J=10.8 Hz, 1H), 5.45 (d, J=2.0 Hz, 1H), 5.20 (dt, J=10.8, 5.4 Hz, 1H), 5.15 (dd, J=14.8, 9.0 Hz, 1H), 4.98 (dd, J=8.4, 1.4 Hz, 1H), 4.79 (dd, J=4.3, 1.0 Hz, 1H), 4.21 (ddd, J=10.7, 4.4, 1.8 Hz, 1H), 4.10-4.03 (m, 2H), 3.68 (dd, J=10.4, 2.3 Hz, 1H), 3.65 (s, 3H), 3.31 (td, J=6.8, 6.1, 2.1 Hz, 1H), 3.24 (s, 3H), 2.56 (tt, J=9.0, 7.0 Hz, 1H), 2.42 (dd, J=11.8, 4.9 Hz, 1H), 2.22-2.13 (m, 1H), 2.03 (d, J=11.0 Hz, 2H), 1.99 (d, J=1.2 Hz, 3H), 1.99-1.95 (m, 1H), 1.93 (d, J=1.2 Hz, 3H), 1.88 (qd, J=7.5, 2.7 Hz, 2H), 1.76 (ddt, J=16.9, 10.4, 6.5 Hz, 1H), 1.39 (td, J=11.5, 2.0 Hz, 1H), 1.06 (d, J=7.0 Hz, 3H), 1.02 (d, J=7.1 Hz, 3H), 0.98 (d, J=6.8 Hz, 3H), 0.92 (d, J=6.8 Hz, 3H), 0.89 (dd, J=6.7, 1.9 Hz, 6H), 0.85 (d, J=6.8 Hz, 3H). ¹³C NMR (151 MHz, Acetone) δ 167.83, 166.35, 145.72, 144.82, 141.96, 134.45, 134.22, 133.80, 132.80, 131.65, 130.18, 129.38, 127.05, 125.25, 99.83, 83.35, 80.43, 77.43, 76.59, 75.28, 71.62, 60.18, 55.68, 43.00, 42.28, 41.71, 40.81, 39.16, 38.30, 38.01, 28.80, 22.26, 21.65, 20.36, 17.74, 14.65, 14.17, 12.71, 10.31, 7.39.

### Example 7: 21-pent-3-enoate Bafilomycin (Compound 7)

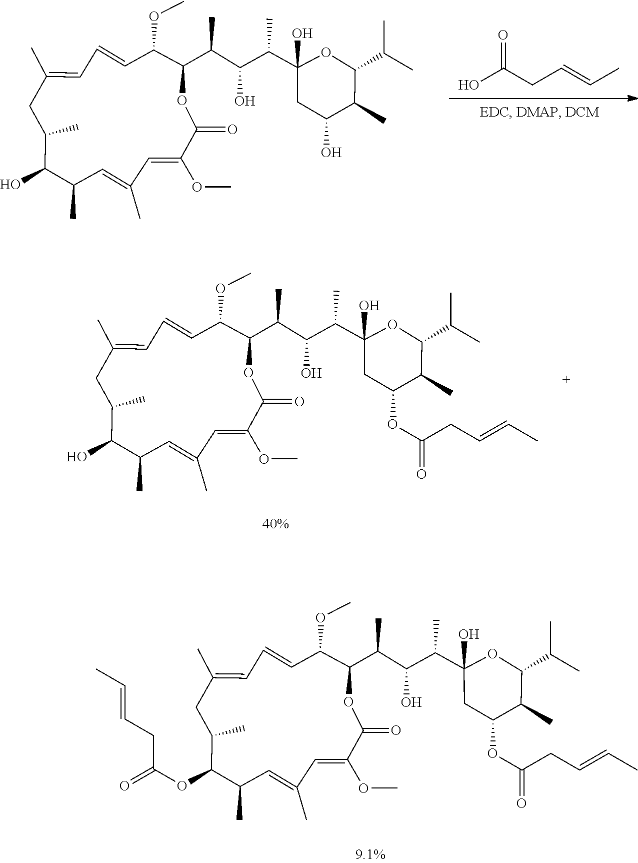

(E)-pent-3-enoic acid (4.18 mg, 41.7 μmol, 2 eq) was added to a solution of bafilomycin A₁ (13.0 mg, 20.9 μmol) in DCM (2.36 mL) under anhydrous conditions. DMAP (5.35 mg, 43.8 μmol, 2.1 eq) and EDC (8.80 mg, 45.9 μmol, 2.2 eq) were added to the solution and stirred overnight. The reaction mixture was concentrated and purified by pTLC (20% acetone in hexanes) to yield 21-pent-3-enoate Bafilomycin and 7,21-dipent-3-enoate Bafilomycin. 21-pent-3-enoate Bafilomycin (Compound 7) ¹H NMR (599 MHz, Acetone) δ 6.71 (s, 1H), 6.68 (dd, J=14.9, 10.8 Hz, 1H), 5.96 (d, J=8.8 Hz, 1H), 5.80 (d, J=10.7 Hz, 1H), 5.65-5.51 (m, 2H), 5.38 (d, J=2.1 Hz, 1H), 5.14 (dd, J=15.0, 9.1 Hz, 1H), 4.97 (dd, J=8.4, 1.5 Hz, 1H), 4.92 (td, J=10.9, 4.8 Hz, 1H), 4.76 (dd, J=4.3, 1.2 Hz, 1H), 4.18 (ddd, J=10.8, 4.4, 1.8 Hz, 1H), 4.10-4.03 (m, 2H), 3.64 (d, J=1.2 Hz, 3H), 3.58 (dd, J=10.4, 2.2 Hz, 1H), 3.35-3.28 (m, 1H), 3.24 (d, J=1.2 Hz, 3H), 3.06-2.96 (m, 2H), 2.84 (s, 1H), 2.60-2.52 (m, 1H), 2.25 (dd, J=11.7, 4.8 Hz, 1H), 2.20-2.12 (m, 1H), 2.02 (d, J=11.0 Hz, 2H), 1.98 (d, J=1.4 Hz, 3H), 1.93 (s, 3H), 1.96 -1.86 (m, 1H), 1.83 (q, J=7.1 Hz, 1H), 1.68-1.64 (m, 3H), 1.59-1.49 (m, 1H), 1.25-1.18 (m, 1H), 1.05 (d, J=6.9 Hz, 3H), 0.99 (d, J=7.2 Hz, 3H), 0.95 (d, J=6.8 Hz, 3H), 0.92 (d, J=6.7 Hz, 3H), 0.88 (d, J=6.8 Hz, 3H), 0.80 (dd, J=6.7, 1.5 Hz, 6H). ¹³C NMR (151 MHz, Acetone) δ 171.66, 167.81, 145.71, 144.82, 141.95, 134.45, 134.20, 132.79, 129.21, 127.03, 125.24, 124.41, 99.72, 83.34, 80.42, 77.41, 76.55, 74.33, 71.57, 60.17, 55.67, 42.94, 42.27, 41.70, 40.76, 39.02, 38.67, 38.28, 38.01, 28.73, 22.25, 21.62, 20.36, 18.00, 17.73, 14.60, 14.16, 12.53, 10.29, 7.38.

### Example 8: 21-penta-2,4-dienoate Bafilomycin (Compound 8)

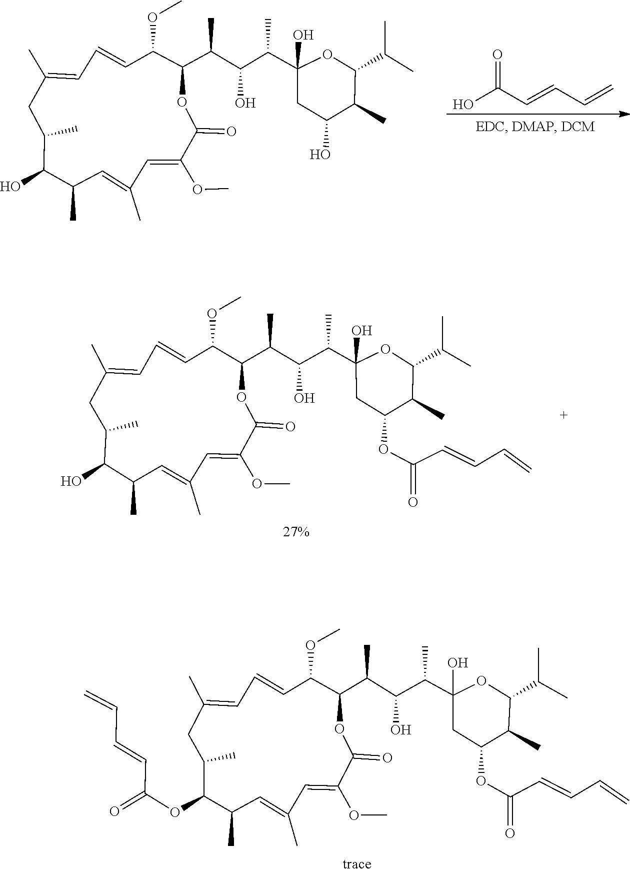

(E)-penta-2,4-dienoic acid (4.73 mg, 48.2 μmol, 2 eq) was added to a solution of bafilomycin A₁ (15.0 mg, 24.1 μmol) in DCM (2.94 mL) under anhydrous conditions. DMAP (6.18 mg, 50.6 μmol, 2.1 eq) and EDC (10.2 mg, 53.0 μmol, 2.2 eq) were added to the solution and stirred overnight. The reaction mixture was concentrated and purified by pTLC (20% acetone in hexanes) to yield 21-penta-2,4-dienoate Bafilomycin and double-addition bafilomycin. 21-penta-2,4-dienoate Bafilomycin (Compound 8) ¹H NMR (599 MHz, Acetone) δ 7.27 (dd, J=15.4, 11.0 Hz, 1H), 6.72 (d, J=1.0 Hz, 1H), 6.68 (dd, J=15.0, 10.8 Hz, 1H), 6.58 (dt, J=17.0, 10.5 Hz, 1H), 6.02-5.96 (m, 1H), 5.96 (d, J=9.1 Hz, 1H), 5.80 (d, J=10.8 Hz, 1H), 5.73-5.67 (m, 1H), 5.52 (dd, J=10.2, 1.6 Hz, 1H), 5.41 (t, J=1.8 Hz, 1H), 5.15 (dd, J=15.0, 9.2 Hz, 1H), 5.01 (td, J=10.9, 4.8 Hz, 1H), 4.97 (dd, J=8.3, 1.5 Hz, 1H), 4.77 (dd, J=4.4, 1.1 Hz, 1H), 4.19 (ddd, J=10.8, 4.5, 1.8 Hz, 1H), 4.09-4.03 (m, 2H), 3.64 (d, J=1.3 Hz, 3H), 3.63 -3.59 (m, 1H), 3.31 (td, J=6.4, 5.5, 1.7 Hz, 1H), 3.24 (d, J=1.3 Hz, 3H), 2.56 (p, J=7.9, 7.5 Hz, 1H), 2.30 (dd, J=11.8, 4.9 Hz, 1H), 2.21-2.13 (m, 1H), 2.02 (s, 2H), 1.98 (d, J=1.3 Hz, 3H), 1.94 (s, 1H), 1.93 (d, J=1.4 Hz, 3H), 1.85 (q, J=7.3 Hz, 1H), 1.60 (td, J=10.4, 6.4 Hz, 1H), 1.30-1.23 (m, 1H), 1.05 (d, J=6.9 Hz, 3H), 1.00 (d, J=7.1 Hz, 3H), 0.96 (d, J=6.8 Hz, 2H), 0.92 (d, J=6.7 Hz, 3H), 0.88 (d, J=6.8 Hz, 3H), 0.82 (dd, J=6.7, 3.5 Hz, 6H). ¹³C NMR (151 MHz, Acetone) δ 167.82, 166.52, 145.71, 145.34, 144.82, 141.95, 135.91, 134.45, 134.21, 132.79, 127.04, 125.95, 125.24, 123.44, 99.75, 83.35, 80.42, 77.42, 76.56, 74.38, 71.59, 60.17, 55.67, 42.98, 42.27, 41.70, 40.82, 39.09, 38.29, 38.01, 28.76, 22.25, 21.63, 20.36, 17.73, 14.61, 14.16, 12.58, 10.30, 7.37.

### Example 9: 7-one Bafilomycin & 7,21-dione Bafilomycin (Compounds 9 and 10)

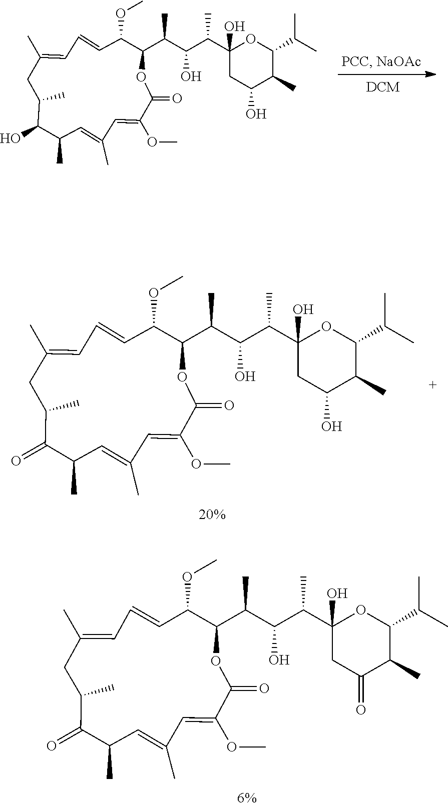 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; CO/C1=C\C(C)=C\[C@@H](C)C(=O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; CO/C1=C\C(C)=C\[C@@H](C)C(=O)[C@@H](C)C/C(C)=C/C=C/[C@H](OC)[C@@H]([C@@H](C)[C@@H](O)[C@H](C)[C@@]2(O)CC(=O)[C@H](C)[C@@H](C(C)C)O2)OC1=O]

Bafilomycin A₁ (32.0 mg, 51.4 μmol) was dissolved in DCM (6.61 mL) under anhydrous conditions then cooled to 0° C. PCC (65.3 mg, 303. μmol, 5.9 eq) and NaOAc (23.4 mg, 285 μmol) were then added and the reaction was stirred for 75 min maintaining the 0° C. temperature. Ether was added to the reaction flask (˜40 mL) then left to stir for 15 minutes at 0° C. The reaction mixture was filtered over Celite® using ether then was concentrated before repeating the filtering and concentrating process again. The crude material was purified by FCC (10-100% Ethyl acetate/Hexanes). Both single and double oxidized products were isolated. The single oxidized product (7-one bafilomycin) was further purified by pTLC using 40% EA/Hex mixture to yield 7,21-dione bafilomycin. 7-one Bafilomycin (Compound 9): ¹H NMR (599 MHz, CDCl₃) δ 6.51 (s, 1H), 6.50-6.45 (m, 1H), 5.84 (d, J=11.0 Hz, 1H), 5.49 (d, J=2.1 Hz, 1H), 5.26-5.21 (m, 1H), 5.24-5.18 (m, 1H), 4.94 (dd, J=9.4, 1.4 Hz, 1H), 4.58 (dd, J=4.1, 1.2 Hz, 1H), 4.13 (ddd, J=10.8, 4.1, 2.0 Hz, 1H), 3.86 (t, J=9.4 Hz, 1H), 3.73-3.66 (m, 1H), 3.65 (s, 3H), 3.49 (dd, J=10.3, 2.2 Hz, 1H), 3.42 (dq, J=11.0, 6.6 Hz, 1H), 3.25 (s, 3H), 2.80 (ddd, J=11.2, 6.9, 4.0 Hz, 1H), 2.33 -2.26 (m, 2H), 2.27-2.19 (m, 1H), 2.15 (dd, J=12.6, 3.5 Hz, 1H), 2.08 (d, J=1.4 Hz, 3H), 1.88 (pd, J=6.8, 2.2 Hz, 1H), 1.80-1.75 (m, 1H), 1.75-1.71 (m, 3H), 1.38-1.28 (m, 1H), 1.16 (td, J=11.6, 2.1 Hz, 1H), 1.08 (d, J=6.6 Hz, 3H), 1.05 (d, J=7.2 Hz, 3H), 1.02 (d, J=6.8 Hz, 3H), 0.99 (s, 0H), 0.94 (d, J=6.5 Hz, 3H), 0.90 (d, J=6.8 Hz, 3H), 0.84 (d, J=6.9 Hz, 3H), 0.77 (d, J=6.8 Hz, 3H). ¹³C NMR (151 MHz, CDCl₃) δ 214.57, 166.78, 142.59, 140.56, 137.83, 134.83, 133.24, 131.67, 128.88, 126.57, 98.93, 81.73, 76.61, 75.84, 70.96, 70.52, 59.78, 55.89, 46.56, 45.53, 43.55, 43.09, 42.18, 41.04, 36.26, 27.90, 21.26, 19.43, 19.32, 14.36, 14.05, 13.64, 12.15, 9.62, 7.06. 7,21-dione Bafilomycin (Compound 10): ¹H NMR (599 MHz, CDCl₃) δ 6.52 (s, 1H), 6.49 (dd, J=15.0, 11.0 Hz, 1H), 5.85 (d, J=10.9 Hz, 1H), 5.67 (d, J=2.4 Hz, 1H), 5.24 (d, J=10.4 Hz, 1H), 5.23-5.18 (m, 1H), 4.93 (d, J=9.4 Hz, 1H), 4.68 (d, J=4.0 Hz, 1H), 4.18-4.12 (m, 1H), 3.87 (t, J=9.4 Hz, 1H), 3.81 (dd, J=10.5, 2.3 Hz, 1H), 3.67 (s, 3H), 3.43 (dq, J=10.6, 6.6 Hz, 1H), 3.25 (s, 3H), 2.81 (ddd, J=11.3, 7.0, 4.1 Hz, 1H), 2.74 (d, J=13.4 Hz, 1H), 2.38 - 2.31 (m, 1H), 2.27 (ddd, J=18.8, 8.8, 4.8 Hz, 3H), 2.15 (dd, J=12.7, 3.8 Hz, 1H), 2.09 (s, 3H), 1.96 (q, J=7.4 Hz, 1H), 1.89 (qt, J=7.0, 3.6 Hz, 1H), 1.74 (s, 3H), 1.08 (d, J=6.6 Hz, 3H), 1.03 (d, J=7.0 Hz, 6H), 0.97 (t, J=6.2 Hz, 6H), 0.87 (t, J=6.3 Hz, 6H). ¹³C NMR (151 MHz, CDCl₃) δ 214.51, 208.87, 166.90, 142.51, 140.68, 138.01, 134.81, 133.35, 131.90, 128.76, 126.54, 101.19, 81.68, 77.02, 76.61, 70.62, 59.82, 55.89, 50.93, 47.35, 46.59, 45.54, 43.07, 41.90, 36.24, 28.75, 21.08, 19.45, 19.32, 14.18, 14.04, 13.64, 9.58, 8.88, 6.91.

### Example 10: 21-pent-4-ynoate Bafilomycin (Compound 11)

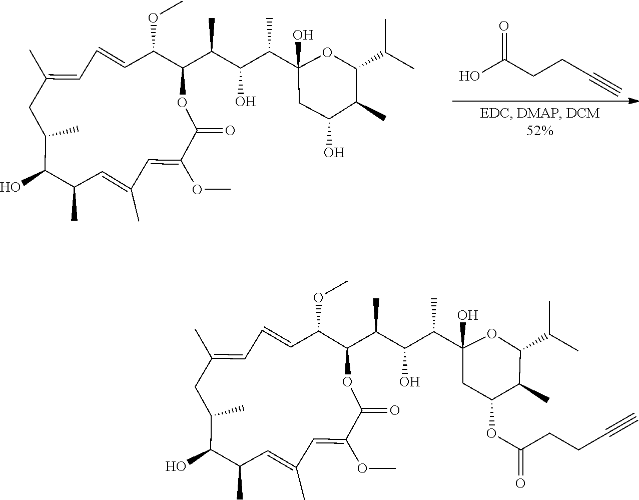

Pent-4-ynoic acid (2.26 mg, 23.1 μmol, 1 eq) was added to a solution of bafilomycin A₁ (14.0 mg, 23.1 μmol) in DCM (2.67 mL) under anhydrous conditions. DMAP (5.9 mg, 48.5 μmol, 2.1 eq) and EDC (9.7 mg, 50.8 μmol, 2.2 eq) were added to the solution and stirred overnight. The reaction mixture was concentrated and purified by pTLC (20% acetone in hexanes) to yield the desired product. 21-pent-4-ynoate Bafilomycin (Compound 11) 1 H NMR (599 MHz, Acetone) δ 6.71 (s, 1H), 6.68 (dd, J=15.0, 10.8 Hz, 1H), 5.96 (d, J=8.9 Hz, 1H), 5.80 (d, J=10.9 Hz, 1H), 5.39 (d, J=2.1 Hz, 1H), 5.14 (dd, J=15.0, 9.2 Hz, 1H), 5.01-4.93 (m, 2H), 4.76 (dd, J=4.4, 1.1 Hz, 1H), 4.18 (ddd, J=10.7, 4.4, 1.8 Hz, 1H), 4.09-4.03 (m, 2H), 3.64 (d, J=0.7 Hz, 3H), 3.59 (dd, J=10.3, 2.3 Hz, 1H), 3.31 (td, J=6.3, 5.4, 1.8 Hz, 1H), 3.24 (d, J=0.7 Hz, 3H), 2.59-2.51 (m, 3H), 2.51-2.44 (m, 2H), 2.37 (t, J=2.6 Hz, 1H), 2.27 (dd, J=11.8, 4.8 Hz, 1H), 2.16 (dd, J=10.5, 6.9 Hz, 1H), 2.09-2.00 (m, 2H), 1.98 (d, J=1.2 Hz, 3H), 1.93 (s, 3H), 1.93-1.86 (m, 2H), 1.84 (q, J=7.7, 7.1 Hz, 1H), 1.55 (tdd, J=12.9, 8.4, 5.2 Hz, 1H), 1.24 (td, J=11.5, 2.1 Hz, 1H), 1.05 (d, J=7.0 Hz, 3H), 0.99 (d, J=7.1 Hz, 3H), 0.95 (d, J=6.9 Hz, 3H), 0.92 (d, J=6.8 Hz, 3H), 0.88 (d, J=6.8 Hz, 3H), 0.83 (d, J=6.5 Hz, 3H), 0.80 (d, J=6.8 Hz, 3H). ¹³C NMR (151 MHz, Acetone) δ 171.65, 167.82, 145.72, 144.83, 141.95, 134.46, 134.21, 132.79, 127.03, 125.24, 99.73, 83.51, 83.35, 80.42, 77.41, 76.58, 74.60, 71.58, 70.40, 60.17, 55.67, 42.95, 42.27, 41.71, 40.79, 38.98, 38.29, 38.01, 34.26, 28.74, 22.25, 21.63, 20.36, 17.73, 14.88, 14.59, 14.16, 12.59, 10.30, 7.38.

### Example 11: 21-(3-(3-(but-3-yn-1-yl)-3H-diazirin-3-yl)propanoate) Bafilomycin (Compound 12)

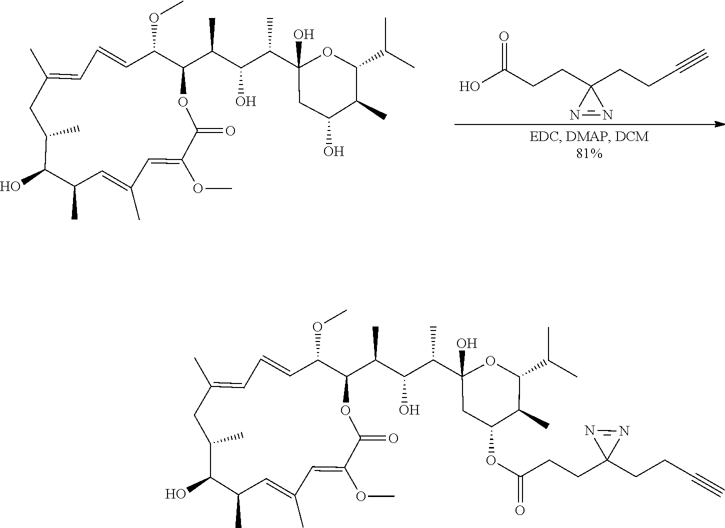 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; C#CCCC1(CCC(=O)O)N=N1 ; C#CCCC1(CCC(=O)O[C@@H]2C[C@](O)([C@@H](C)[C@H](O)[C@H](C)[C@H]3OC(=O)/C(OC)=C/C(C)=C/[C@@H](C)[C@@H](O)[C@@H](C)C/C(C)=C/C=C/[C@@H]3OC)O[C@H](C(C)C)[C@H]2C)N=N1]

3-(3-(but-3-yn-1-yl)-3H-diazirin-3-yl)propanoic acid (8.0 mg, 48.2 μmol, 2 eq) was added to a solution of bafilomycin A₁ (15.0 mg, 24.1 μmol) in DCM (2.94 mL) under anhydrous conditions. DMAP (6.18 mg, 50.6 μmol, 2.1 eq) and EDC (10.2 mg, 53.0 μmol, 2.2 eq) were added to the solution and stirred overnight. The reaction mixture was concentrated and purified by pTLC (20% acetone in hexanes) to yield the desired product. 21-(3-(3-(but-3-yn-1-yl)-3H-diazirin-3-yl)propanoate) Bafilomycin (Compound 12) ¹H NMR (599 MHz, Acetone) δ 6.71 (d, J=0.9 Hz, 1H), 6.68 (dd, J=15.0, 10.8 Hz, 1H), 5.96 (dt, J=8.8, 1.2 Hz, 1H), 5.83-5.78 (m, 1H), 5.39 (d, J=2.1 Hz, 1H), 5.14 (dd, J=15.0, 9.2 Hz, 1H), 4.99-4.94 (m, 1H), 4.97-4.91 (m, 1H), 4.76 (dd, J=4.4, 1.1 Hz, 1H), 4.18 (ddd, J=10.7, 4.4, 1.9 Hz, 1H), 4.09-4.02 (m, 2H), 3.64 (s, 3H), 3.58 (dd, J=10.4, 2.2 Hz, 1H), 3.31 (ddd, J=7.3, 5.5, 2.0 Hz, 1H), 3.24 (s, 3H), 2.60-2.51 (m, 1H), 2.39 (t, J=2.7 Hz, 1H), 2.28 (dd, J=11.8, 4.8 Hz, 1H), 2.18 (td, J=7.5, 3.5 Hz, 2H), 2.15 (ddt, J=6.7, 4.3, 2.0 Hz, 1H), 2.09-2.05 (m, 2H), 2.05-2.00 (m, 2H), 1.98 (d, J=1.2 Hz, 3H), 1.96-1.85 (m, 5H), 1.85-1.81 (m, 1H), 1.81-1.77 (m, 2H), 1.66 (t, J=7.5 Hz, 2H), 1.54 (tdd, J=13.1, 8.6, 5.3 Hz, 1H), 1.23 (td, J=11.5, 2.1 Hz, 1H), 1.05 (d, J=7.0 Hz, 3H), 0.99 (d, J=7.2 Hz, 3H), 0.95 (d, J=6.8 Hz, 3H), 0.92 (d, J=6.8 Hz, 3H), 0.87 (dd, J=7.0, 2.8 Hz, 3H), 0.82 (d, J=6.5 Hz, 3H), 0.80 (d, J=6.8 Hz, 3H). ¹³C NMR (151 MHz, Acetone) δ 172.27, 167.95, 145.85, 144.96, 142.08, 134.59, 134.34, 132.92, 127.17, 125.37, 99.86, 83.66, 83.47, 80.56, 77.55, 76.70, 74.75, 71.71, 70.73, 60.30, 55.80, 43.08, 42.41, 41.84, 40.87, 39.11, 38.40, 38.14, 33.10, 29.22, 28.87, 28.83, 28.70, 22.39, 21.76, 20.50, 17.86, 14.73, 14.30, 13.72, 12.72, 10.43, 7.53.

### Example 12: 21-(3-(trifluoromethyl)-3H-diazirin-3-yl)benzoate Bafilomycin (Compound 13)

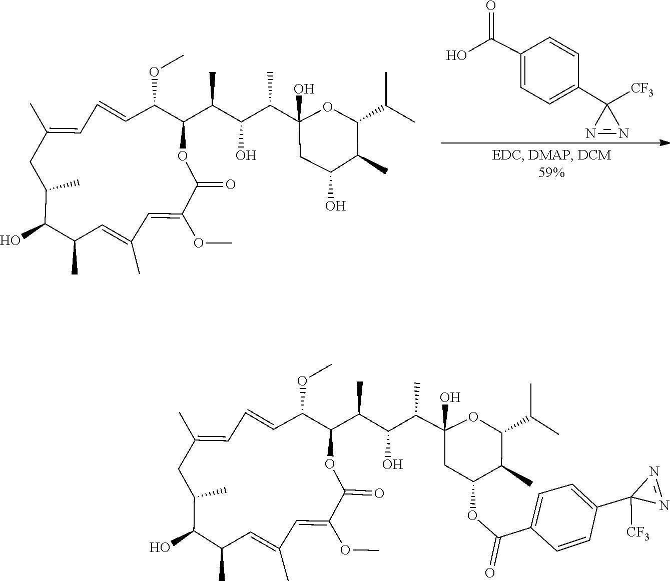 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; O=C(O)c1ccc(C2(C(F)(F)F)N=N2)cc1 ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)c3ccc(C4(C(F)(F)F)N=N4)cc3)[C@H](C)[C@@H](C(C)C)O2)OC1=O]

4-(3-(trifluoromethyl)-3H-diazirin-3-yl)benzoic acid (15.2 mg, 65.92 μmol, 2 eq) was added to a solution of 21-pent-4-ynoate Bafilomycin (20.0 mg, 33.0 μmol) in DCM (4.10 mL) under anhydrous conditions. DMAP (8.46 mg, 69.2 μmol, 2.1 eq) and EDC (13.3 mg, 69.2 μmol, 2.2 eq) were added to the solution and stirred overnight. The reaction mixture was concentrated and purified by pTLC (20% acetone in hexanes) to yield the desired product. 21-(3-(trifluoromethyl)-3H-diazirin-3-yl)benzoate Bafilomycin (Compound 13)

¹H NMR (599 MHz, Acetone) δ 8.18-8.13 (m, 2H), 7.44 (d, J=8.2 Hz, 2H), 6.72 (d, J=0.9 Hz, 1H), 6.68 (dd, J=15.0, 10.8 Hz, 1H), 5.97 (dt, J=8.9, 1.2 Hz, 1H), 5.81 (d, J=10.8 Hz, 1H), 5.47 (d, J=2.0 Hz, 1H), 5.20 (td, J=10.9, 4.8 Hz, 1H), 5.15 (dd, J=15.0, 9.2 Hz, 1H), 4.98 (dd, J=8.4, 1.4 Hz, 1H), 4.79 (dd, J=4.4, 1.0 Hz, 1H), 4.21 (ddd, J=10.7, 4.4, 1.9 Hz, 1H), 4.09-4.03 (m, 2H), 3.70-3.62 (m, 1H), 3.65 (s, 3H), 3.34-3.28 (m, 1H), 3.24 (s, 3H), 2.56 (tt, J=7.2, 5.1 Hz, 1H), 2.41 (dd, J=11.8, 4.9 Hz, 1H), 2.22-2.14 (m, 1H), 2.03 (d, J=11.0 Hz, 2H), 1.99 (d, J=1.2 Hz, 3H), 1.97 (td, J=6.7, 2.1 Hz, 1H), 1.93 (d, J=1.2 Hz, 3H), 1.92-1.85 (m, 2H), 1.81 -1.71 (m, 1H), 1.40 (td, J=11.5, 2.1 Hz, 1H), 1.06 (d, J=7.0 Hz, 3H), 1.01 (d, J=7.2 Hz, 3H), 0.98 (d, J=6.9 Hz, 3H), 0.92 (d, J=6.8 Hz, 3H), 0.89 (d, J=4.2 Hz, 3H), 0.88 (d, J=3.8 Hz, 3H), 0.85 (d, J=6.8 Hz, 3H). ¹³C NMR (151 MHz, Acetone) δ 168.00, 167.97, 165.54, 145.87, 144.97, 142.09, 134.60, 134.37, 133.95, 133.23, 132.93, 131.04, 127.70, 127.17, 125.38, 124.00, 122.18, 99.97, 83.48, 80.56, 77.56, 76.69, 76.12, 71.75, 60.32, 55.81, 49.93, 43.11, 42.41, 41.85, 40.85, 39.24, 38.42, 38.15, 28.92, 22.39, 21.77, 20.50, 17.87, 14.77, 14.30, 12.81, 10.44, 7.52.

### Example 13: 21-2-(2-(prop-2-yn-1-yloxy)ethoxy)-4-(3-(trifluoromethyl)-3H-diazirin-3-yl)benzoate Bafilomycin (Compound 14)

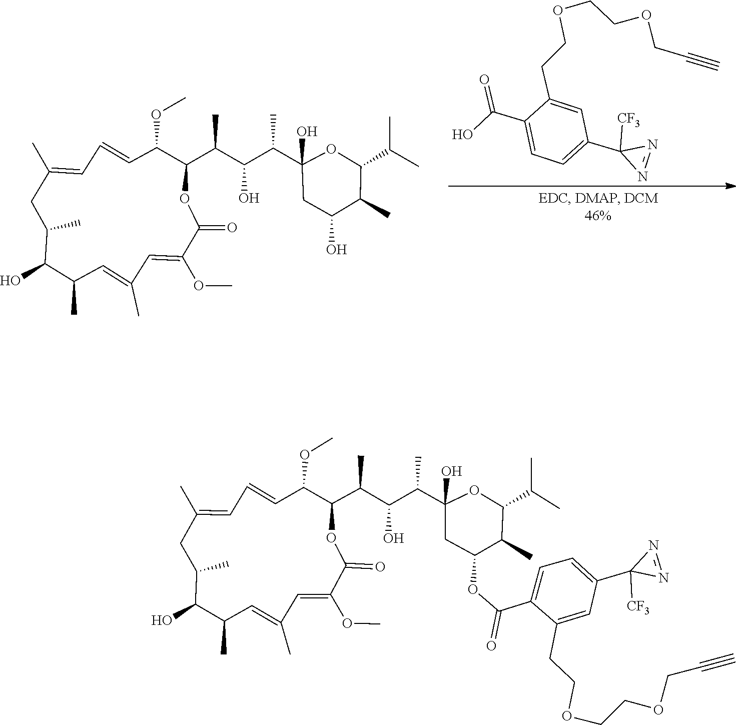 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; C#CCOCCOCCc1cc(C2(C(F)(F)F)N=N2)ccc1C(=O)O ; C#CCOCCOCCc1cc(C2(C(F)(F)F)N=N2)ccc1C(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C]

2-(2-(prop-2-yn-1-yloxy)ethoxy)-4-(3-(trifluoromethyl)-3H-diazirin-3-yl)benzoic acid (16.0 mg, 49.4 μmol, 2 eq) was added to a solution of bafilomycin A₁ (15.0 mg, 24.7 μmol) in DCM (3.02 mL) under anhydrous conditions. DMAP (6.34 mg, 51.9 μmol, 2.1 eq) and EDC (9.95 mg, 51.9 μmol, 2.1 eq) were added to the solution and stirred overnight. The reaction mixture was concentrated and purified by pTLC (20% acetone in hexanes) to yield the desired product. 21-2-(2-(prop-2-yn-1-yloxy)ethoxy)-4-(3-(trifluoromethyl)-3H-diazirin-3-yl)benzoate Bafilomycin (Compound 14) ¹H NMR (599 MHz, Acetone) δ 7.79 (d, J=8.1 Hz, 1H), 7.02 (ddt, J=8.1, 1.8, 0.9 Hz, 1H), 6.91 (d, J=1.7 Hz, 1H), 6.72 (d, J=0.9 Hz, 1H), 6.68 (dd, J=15.0, 10.8 Hz, 1H), 5.96 (dt, J=9.0, 1.2 Hz, 1H), 5.81 (d, J=10.8 Hz, 1H), 5.45 (d, J=2.1 Hz, 1H), 5.23-5.12 (m, 2H), 4.98 (dd, J=8.4, 1.4 Hz, 1H), 4.78 (dd, J=4.4, 1.1 Hz, 1H), 4.32-4.27 (m, 2H), 4.27 (d, J=2.4 Hz, 2H), 4.21 (ddd, J=10.7, 4.4, 1.9 Hz, 1H), 4.09-4.03 (m, 2H), 3.93-3.88 (m, 2H), 3.69-3.63 (m, 1H), 3.65 (s, 3H), 3.31 (ddd, J=7.3, 5.5, 2.0 Hz, 1H), 3.24 (s, 3H), 2.95 (t, J=2.4 Hz, 1H), 2.60-2.52 (m, 1H), 2.41 (dd, J=11.8, 4.8 Hz, 1H), 2.22-2.15 (m, 1H), 2.03 (d, J=11.0 Hz, 1H), 1.99 (d, J=1.2 Hz, 3H), 1.96 (td, J=6.8, 2.2 Hz, 1H), 1.93 (d, J=1.1 Hz, 3H), 1.92-1.85 (m, 2H), 1.69 (tdd, J=13.0, 8.5, 5.2 Hz, 1H), 1.42-1.35 (m, 1H), 1.05 (d, J=7.0 Hz, 3H), 1.02 (d, J=7.1 Hz, 3H), 0.99-0.93 (m, 7H), 0.92 (d, J=6.6 Hz, 6H), 0.88 (d, J=6.9 Hz, 3H), 0.83 (d, J=6.8 Hz, 3H).

### Example 14: 21-(3-(trifluoromethyl)-3H-diazirin-3-yl)benzoate-7-pent-4-ynoate-Bafilomycin (Compound 15)

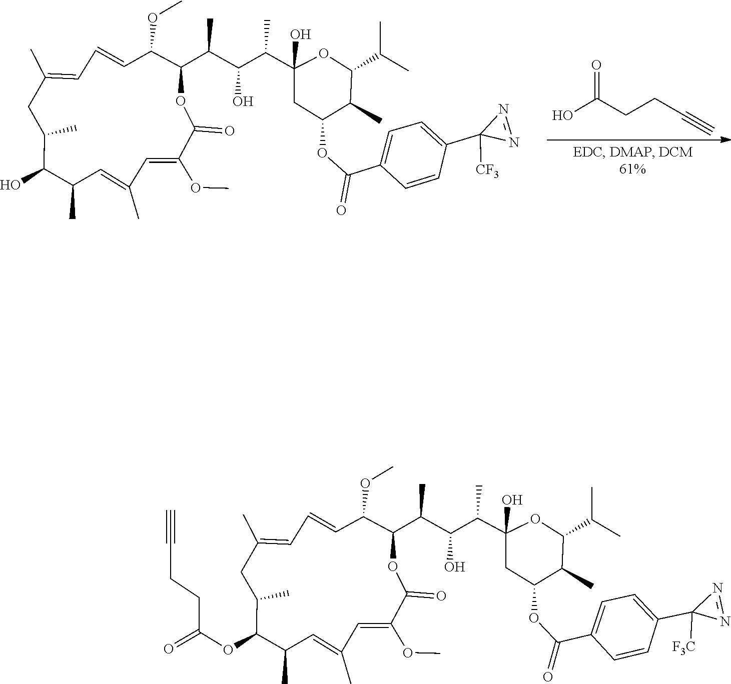

Pent-4-ynoate acid (2.87 mg, 29.3 μmol, 2 eq) was added to a solution of 21-(3-(trifluoromethyl)-3H-diazirin-3-yl)benzoate Bafilomycin (12.0 mg, 14.7 μmol) in DCM (1.83 mL) under anhydrous conditions. DMAP (3.76 mg, 30.8 μmol, 2.1 eq) and EDC (5.90 mg, 30.8 μmol, 2.1 eq) were added to the solution and stirred overnight. The reaction mixture was concentrated and purified by pTLC (20% acetone in hexanes) to yield the desired product. 21-(3-(trifluoromethyl)-3H-diazirin-3-yl)benzoate-7-pent-4-ynoate-Bafilomycin (Compound 15) ¹H NMR (599 MHz, Acetone) δ 8.18 (dq, J=8.5, 1.9 Hz, 2H), 7.50-7.45 (m, 2H), 6.81-6.77 (m, 1H), 6.68 (dd, J=15.1, 10.6 Hz, 1H), 6.05 (dt, J=9.0, 1.3 Hz, 1H), 5.96 (d, J=10.5 Hz, 1H), 5.38 (d, J=2.1 Hz, 1H), 5.28 (dd, J=15.0, 9.0 Hz, 1H), 5.02 (dt, J=8.3, 1.6 Hz, 1H), 4.98 (td, J=11.0, 4.9 Hz, 1H), 4.95 (dd, J=6.8, 2.2 Hz, 1H), 4.74-4.70 (m, 1H), 4.25-4.19 (m, 1H), 4.09 (t, J=8.7 Hz, 1H), 3.68 (d, J=1.6 Hz, 3H), 3.60 (dd, J=10.4, 2.2 Hz, 1H), 3.22 (s, 3H), 2.94 (ddd, J=9.1, 7.0, 2.2 Hz, 1H), 2.58-2.51 (m, 2H), 2.52-2.45 (m, 2H), 2.38 (t, J=2.6 Hz, 1H), 2.28 (ddd, J=11.8, 4.9, 1.7 Hz, 1H), 2.26-2.13 (m, 3H), 2.04 (s, 3H), 1.98-1.89 (m, 1H), 1.86 (q, J=6.9 Hz, 1H), 1.77 (dd, J=15.4, 11.6 Hz, 1H), 1.62-1.53 (m, 1H), 1.52 (d, J=1.4 Hz, 3H), 1.25 (td, J=11.5, 2.0 Hz, 1H), 1.14 (d, J=6.8 Hz, 3H), 1.02-0.98 (m, 3H), 1.01-0.96 (m, 3H), 0.96 (dd, J=6.9, 1.6 Hz, 3H), 0.93 (d, J=6.9 Hz, 3H), 0.86-0.82 (m, 3H), 0.82 (d, J=6.8 Hz, 3H). ¹³C NMR (151 MHz, Acetone) δ 171.66, 167.59, 166.53, 142.98, 142.79, 141.53, 134.33, 134.17, 133.58, 133.17, 132.63, 131.24, 128.71, 127.73, 126.07, 123.85, 122.31, 99.74, 84.12, 83.52, 83.20, 77.80, 76.61, 74.59, 71.59, 70.41, 60.19, 55.76, 42.99, 41.82, 40.79, 39.11, 38.99, 38.45, 37.13, 34.27, 28.75, 22.03, 21.64, 20.67, 17.47, 14.89, 14.61, 14.23, 12.60, 10.43, 7.36.

### Example 15: 21-pent-4-ynoate-7-(3-(trifluoromethyl)-3H-diazirin-3-yl)benzoate Bafilomycin (Compound 16)

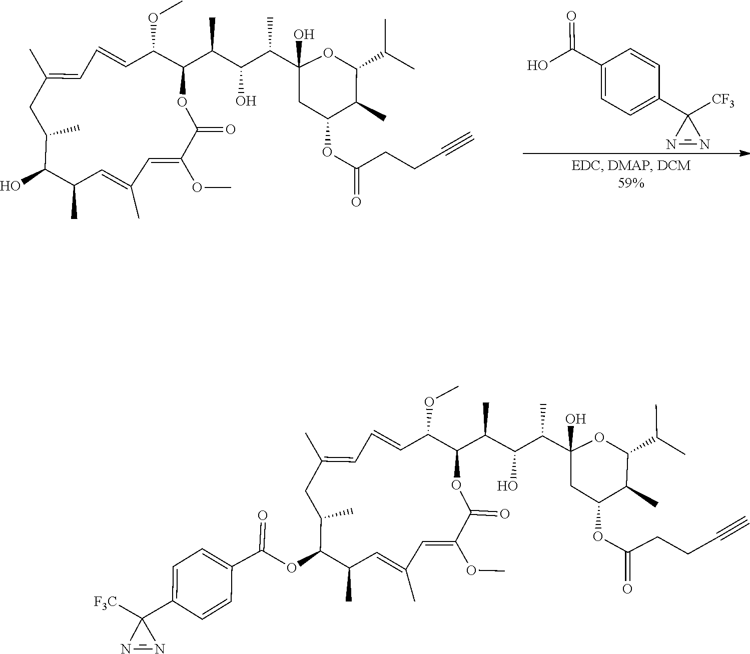 [SMILES: C#CCCC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C ; O=C(O)c1ccc(C2(C(F)(F)F)N=N2)cc1 ; C#CCCC(=O)O[C@@H]1C[C@](O)([C@@H](C)[C@H](O)[C@H](C)[C@H]2OC(=O)/C(OC)=C/C(C)=C/[C@@H](C)[C@@H](OC(=O)c3ccc(C4(C(F)(F)F)N=N4)cc3)[C@@H](C)C/C(C)=C/C=C/[C@@H]2OC)O[C@H](C(C)C)[C@H]1C]

4-(3-(trifluoromethyl)-3H-diazirin-3-yl)benzoic acid (7.8 mg, 34.1 μmol, 2 eq) was added to a solution of bafilomycin A₁ (12.0 mg, 17.1 μmol) in DCM (2.12 mL) under anhydrous conditions. DMAP (4.38 mg, 35.9 μmol, 2.1 eq) and EDC (6.87 mg, 35.9 μmol, 2.1 eq) were added to the solution and stirred overnight. The reaction mixture was concentrated and purified by pTLC (20% acetone in hexanes) to yield the desired product. 21-pent-4-ynoate-7-(3-(trifluoromethyl)-3H-diazirin-3-yl)benzoate Bafilomycin (Compound 16) ¹H NMR (599 MHz, Acetone) δ 8.21-8.16 (m, 2H), 7.47 (d, J=8.2 Hz, 2H), 6.79 (s, 1H), 6.69 (dd, J=15.1, 10.6 Hz, 1H), 6.05 (d, J=9.0 Hz, 1H), 5.96 (d, J=10.6 Hz, 1H), 5.38 (d, J=2.0 Hz, 1H), 5.28 (dd, J=15.0, 9.1 Hz, 1H), 5.02 (dd, J=8.1, 1.4 Hz, 1H), 4.98 (td, J=11.0, 4.9 Hz, 1H), 4.95 (dd, J=6.7, 2.3 Hz, 1H), 4.72 (d, J=4.4 Hz, 1H), 4.22 (ddd, J=10.7, 4.5, 1.8 Hz, 1H), 4.09 (t, J=8.6 Hz, 1H), 3.68 (s, 3H), 3.60 (dd, J=10.3, 2.2 Hz, 1H), 3.31 (d, J=3.5 Hz, 1H), 3.22 (s, 3H), 3.12-3.08 (m, 1H), 2.94 (tt, J=9.2, 7.0 Hz, 1H), 2.78 (s, 1H), 2.58-2.52 (m, 2H), 2.51-2.45 (m, 2H), 2.38 (t, J=2.6 Hz, 1H), 2.28 (dd, J=11.8, 4.8 Hz, 1H), 2.25-2.13 (m, 3H), 2.06 (s, 3H), 1.98-1.90 (m, 1H), 1.90-1.83 (m, 1H), 1.77 (dd, J=15.3, 11.5 Hz, 1H), 1.62-1.53 (m, 1H), 1.52 (s, 3H), 1.25 (td, J=11.5, 2.0 Hz, 1H), 1.14 (d, J=6.8 Hz, 3H), 1.00 (dd, J=8.8, 7.0 Hz, 6H), 0.96 (d, J=6.8 Hz, 3H), 0.93 (d, J=6.8 Hz, 3H), 0.83 (dd, J=9.2, 6.6 Hz, 6H). ¹³C NMR (151 MHz, Acetone) δ 171.65, 167.59, 166.53, 142.98, 142.79, 141.52, 134.33, 134.17, 133.59, 133.17, 132.63, 131.24, 128.72, 127.73, 126.08, 123.85, 122.03, 99.74, 84.12, 83.52, 83.20, 77.80, 76.60, 74.59, 71.59, 70.41, 60.19, 55.76, 42.99, 42.96, 41.82, 40.79, 39.11, 38.99, 38.45, 37.13, 34.27, 28.75, 22.03, 21.64, 20.67, 17.48, 14.89, 14.61, 14.24, 12.60, 10.43, 7.36.

### Example 16: Concanamycin F (Compound 23)

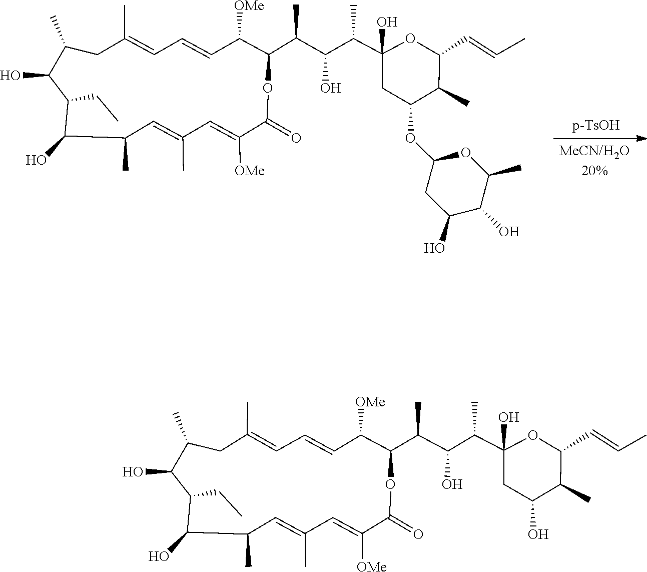 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C ; C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O)[C@@H]1C]

Concanamycin C (CMC; 60.0 mg, 72.9 μmol) was dissolved in MeCN (7.61 mL) and water (1.78 mL) before the addition of pTsOH (55.5, 292 μmol, 4 eq). The reaction was ran overnight (between 15-20 hrs) before being cooled to 0° C. and quenched with a saturated solution of sodium bicarbonate. The crude product was extracted with chloroform (3 times), washed with water, dried over Na2SO4, filtered and concentrated. The crude material was purified by FCC using 4-12% isopropanol in a 25% hexanes/chloroform solution. To fully wash the column, it was flushed with 100% isopropanol. The material collected was further purified by pTLC with 4% isopropanol/chloroform to yield CMF. Concanamycin F (Compound 23) ¹H NMR (599 MHz, CDCl₃, 278 K) δ 6.56 (dd, J=15.1, 10.7 Hz, 1H), 6.40 (s, 1H), 5.85-5.82 (m, 1H), 5.78 (d, J=10.5 Hz, 1H), 5.68 (d, J=9.6 Hz, 1H), 5.55 (dt, J=13.3, 6.8 Hz, 1H), 5.32-5.25 (m, 1H), 5.22 (dd, J=15.2, 9.0 Hz, 1H), 5.02 (d, J=9.0 Hz, 1H), 4.67 (d, J=4.3 Hz, 1H), 4.02 (d, J=11.6 Hz, 1H), 3.97 (t, J=9.2 Hz, 1H), 3.89-3.80 (m, 2H), 3.73 (td, J=10.6, 4.5 Hz, 1H), 3.56 (s, 3H), 3.26 (s, 3H), 3.22 (d, J=10.3 Hz, 1H), 2.73 (s, 1H), 2.32 (dt, J=12.2, 6.1 Hz, 2H), 2.18 (dq, J=9.7, 6.0 Hz, 1H), 2.03 (s, 1H), 1.96 (s, 1H), 1.87 (s, 3H), 1.78-1.73 (m, 4H), 1.71 (s, 4H), 1.63-1.57 (m, 3H), 1.52 (dd, J=10.8, 5.4 Hz, 1H), 1.26 (s, 1H), 1.25 (s, 1H), 1.24-1.12 (m, 2H), 1.09 (d, J=6.7 Hz, 2H), 1.05 (dd, J=7.1, 3.5 Hz, 6H), 0.92 (d, J=6.5 Hz, 3H), 0.90-0.84 (m, 2H), 0.82 (d, J=6.9 Hz, 3H). ¹³C NMR (151 MHz, CDCl₃, 278 K) δ 166.59, 142.11, 141.85, 139.57, 133.28, 132.21, 130.75, 130.58, 127.85, 127.11, 122.98, 99.54, 81.31, 79.65, 75.54, 75.17, 74.29, 70.45, 70.04, 59.06, 55.74, 44.71, 43.53, 43.33, 43.02, 41.30, 36.86, 36.33, 34.57, 29.70, 22.76, 21.67, 17.80, 16.79, 16.39, 14.67, 14.18, 13.22, 11.66, 9.33, 7.07.

### Example 17: 21-deoxy-2-hydroxy Concanamycin F (Compound 24)

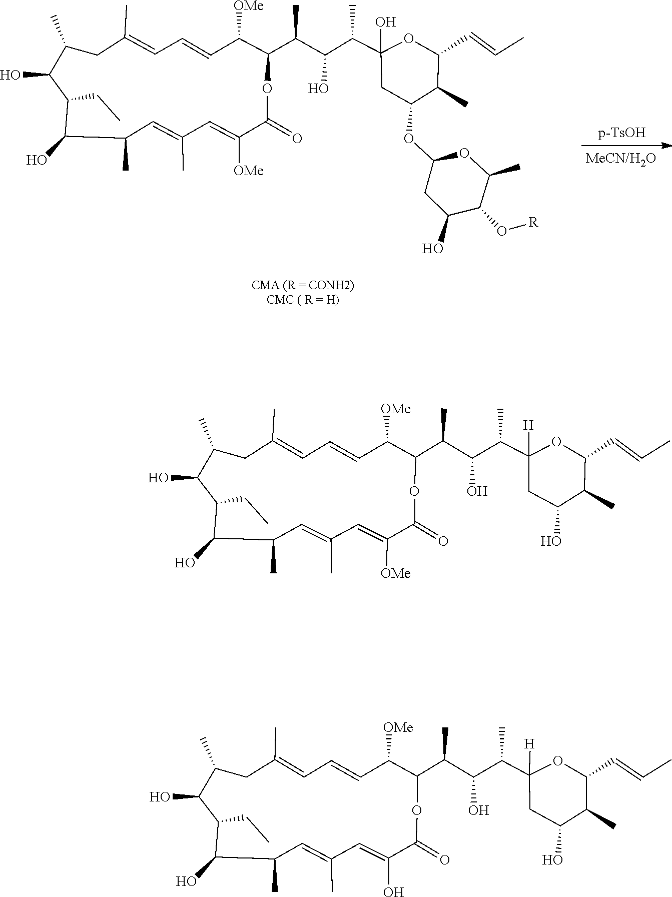

MeCN (11.6 mL) was added to a vial containing a mixture of 21-deoxy concanamycin A and C (140 mg) before water (2.32 mL) was added. The reaction was stirred and pTsOH (72.0 mg, 379 μmol) was added before heating to 38° C. The reaction was run overnight (˜16 hrs) then cooled to 0° C. Sodium bicarbonate was added to the reaction mix to basify and before concentrating. The water mix that was left over was washed with chloroform 3 times. The organics were combined, washed with brine, dried over sodium sulfate, filtered, and concentrated. The crude, yellow foam was first purified by FCC (12% acetone/DCM) and fractions 12/13 were collected and found to contain majority of the product. Starting material was also recovered when the gradient was increased to 100% acetone. Fractions 12/13 were further purified by pTLC (12% acetone/DCM) to yield 21-deoxy concanamycin F (CMF) and 21-deoxy-2-hydroxy-Concanamycin F. 21-deoxy concanamycin F (Compound 23) ¹H NMR (599 MHz, CDCl₃) δ 6.52 (dd, J=15.1, 10.6 Hz, 1H), 6.37 (s, 1H), 5.79 (d, J=10.4 Hz, 1H), 5.68 (s, 1H), 5.62-5.53 (m, 1H), 5.34 (ddd, J=15.3, 7.5, 1.8 Hz, 1H), 5.29-5.24 (m, 1H), 5.23 (s, 2H), 3.84 (t, J=8.9 Hz, 1H), 3.67-3.60 (m, 2H), 3.59 (s, 3H), 3.52-3.45 (m, 1H), 3.40-3.35 (m, 1H), 3.32 (dd, J=9.9, 7.6 Hz, 1H), 3.25 (s, 3H), 2.73 (s, 1H), 2.29 (s, 1H), 2.14-2.05 (m, 2H), 1.98 (s, 4H), 1.84 (s, 4H), 1.74-1.65 (m, 1H), 1.62 (dd, J=6.6, 1.6 Hz, 6H), 1.30-1.17 (m, 3H), 1.07 (s, 5H), 0.91 (s, 5H), 0.89 (dd, J=8.1, 6.8 Hz, 6H), 0.84 (d, J=7.0 Hz, 3H). 21-deoxy-2-hydroxy-Concanamycin F (Compound 24). ¹H NMR (599 MHz, CDCl₃) δ 6.43 (d, J=0.9 Hz, 1H), 6.33 (s, 1H), 5.94 (d, J=9.9 Hz, 1H), 5.79 (d, J=10.8 Hz, 1H), 5.74-5.65 (m, 1H), 5.47-5.40 (m, 2H), 5.08 (t, J=7.7 Hz, 1H), 4.59 (t, J=7.6 Hz, 1H), 3.81 (d, J=10.3 Hz, 1H), 3.67 (d, J=9.3 Hz, 1H), 3.63 (d, J=0.9 Hz, 3H), 3.40 (tdd, J=21.5, 10.7, 6.6 Hz, 3H), 3.24 (d, J=9.5 Hz, 1H), 3.07 (s, 1H), 2.67 (t, J=8.4 Hz, 1H), 2.33 (s, 1H), 2.19-2.09 (m, 3H), 1.95 (s, 3H), 1.88 (d, J=17.6 Hz, 1H), 1.85-1.78 (m, 1H), 1.73 (dd, J=6.4, 1.6 Hz, 3H), 1.68 (s, 3H), 1.48 (d, J=5.3 Hz, 1H), 1.39 (dt, J=15.3, 7.9 Hz, 1H), 1.33-1.22 (m, 1H), 1.25 (s, 3H), 1.13 (d, J=6.9 Hz, 3H), 1.04 (d, J=6.6 Hz, 3H), 0.99 (d, J=7.0 Hz, 3H), 0.98 (s, 0H), 0.95 (d, J=6.6 Hz, 3H), 0.93 (d, J=6.5 Hz, 3H), 0.90 (t, J=7.3 Hz, 3H).

### Example 18: 21-deoxy Concanamycins A-C (Compounds 25-27)

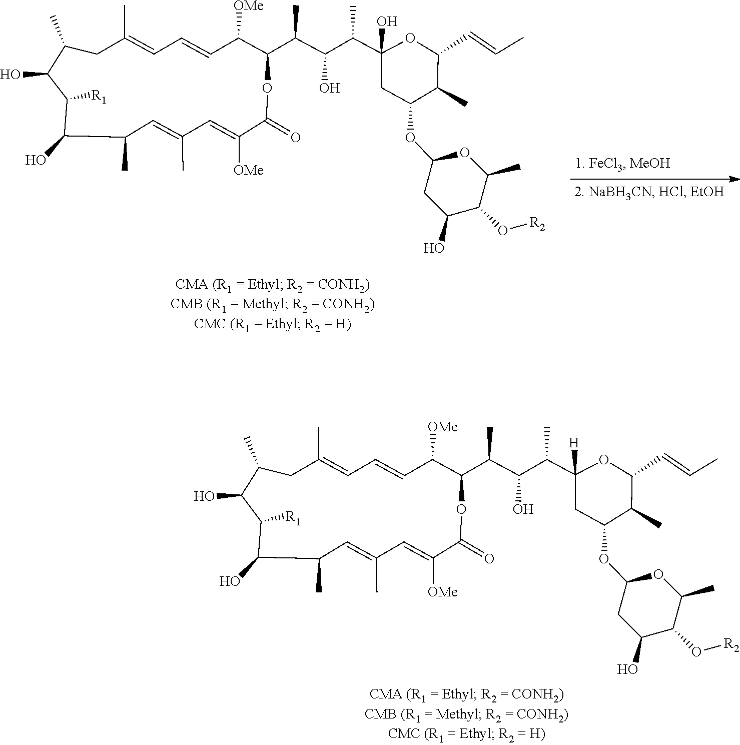

A mixture of concanamycins A-C (111 mg, 128 μmol) was dissolved in MeOH (2.79 mL) and cooled to 0° C. FeCl₃ (4.1575 mg, 25.6 μmol, 0.2 eq) was added to the solution and the reaction was ran for 45 minutes. To neutralize the solution, phosphate buffer (pH 7.1) was added. The solution was extracted with CHCl₃ (3 times), organics were combined, washed with water, brine, dried over Na₂SO₄, filtered, and concentrated. The crude reaction was carried forward to the next reaction without further purification. NaBH₃CN (45.04 mg, 717 μmol) was added to a solution of 21-methoxy-CMA-C (100 mg) in ethanol (9.84 mL). HCl (0.5 M, 987 μL, 493 μmol) was then added. The reaction was allowed to run for 4 hours before it was neutralized with phosphate buffer (pH 7.1). The mixture was extracted with CHCl₃ (3 times), organics were combined, washed with water, brine, dried over Na₂SO₄, filtered and concentrated. The crude material was purified by FCC (15-100% EA/hexanes) to isolate concanamycin material. To separate the concanamycin analogs, pTLC915-100% EA/hexanes) was performed to yield each diastereomer. 21-deoxy CMA (Compound 25). ¹H NMR (599 MHz, CDCl₃) δ 6.56-6.48 (m, 1H), 6.38 (s, 1H), 5.80 (s, 1H), 5.68 (s, 1H), 5.57 (dq, J=13.1, 6.4 Hz, 1H), 5.34 (dd, J=15.1, 7.6 Hz, 1H), 5.29-5.19 (m, 2H), 4.73 (s, 2H), 4.59 (d, J=9.2 Hz, 1H), 4.29 (t, J=9.1 Hz, 1H), 3.84 (t, J=8.9 Hz, 1H), 3.75 (s, 2H), 3.59 (s, 3H), 3.51-3.34 (m, 2H), 3.31 (dd, J=15.8, 7.2 Hz, 1H), 3.25 (s, 3H), 2.99 (s, 1H), 2.74 (s, 1H), 2.29 (s, 1H), 2.22 (dd, J=12.4, 5.1 Hz, 1H), 2.15-2.05 (m, 2H), 2.02-1.97 (m, 4H), 1.85 (s, 3H), 1.74-1.66 (m, 2H), 1.62 (dd, J=6.4, 1.7 Hz, 3H), 1.31 (d, J=16.5 Hz, 1H), 1.31-1.24 (m, 7H), 1.24-1.19 (m, 2H), 1.21-1.15 (m, 1H), 1.07 (s, 4H), 0.91-0.82 (m, 10H).

21-deoxy CMB (Compound 26) ¹H NMR (599 MHz, CDCl₃) δ 6.49-6.39 (m, 1H), 6.34 (dt, J=23.5, 7.0 Hz, 1H), 5.72 (t, J=10.8 Hz, 1H), 5.55-5.46 (m, 1H), 5.32-5.22 (m, 1H), 5.20-5.10 (m, 2H), 5.10-5.03 (m, 2H), 4.52 (d, J=9.2 Hz, 1H), 4.22 (t, J=9.1 Hz, 1H), 3.77 (p, J=9.1 Hz, 1H), 3.75-3.59 (m, 1H), 3.62-3.52 (m, 2H), 3.51 (s, 2H), 3.50 (d, J=3.7 Hz, 0H), 3.44-3.38 (m, 1H), 3.32 (ddd, J=21.5, 10.6, 6.7 Hz, 2H), 3.27-3.22 (m, 1H), 3.18 (d, J=9.4 Hz, 4H), 2.67 (tt, J=16.4, 8.5 Hz, 1H), 2.12 (d, J=16.2 Hz, 1H), 2.05 (d, J=10.6 Hz, 1H), 2.05-1.99 (m, 1H), 1.92 (q, J=15.2, 12.6 Hz, 3H), 1.86-1.78 (m, 0H), 1.78 (s, 2H), 1.62 (q, J=12.3, 11.1 Hz, 2H), 1.54 (d, J=6.2 Hz, 4H), 1.46-1.39 (m, 1H), 1.26-1.13 (m, 7H), 1.14-0.98 (m, 3H), 0.99 (s, 5H), 0.92-0.84 (m, 1H), 0.86-0.77 (m, 12H), 0.80-0.75 (m, 2H), 0.77-0.71 (m, 2H), -0.63 (s, 1H).

21-deoxy CMC (Compound 27): ¹H NMR (599 MHz, CDCl₃) δ 6.52 (dd, J=15.0, 10.8 Hz, 1H), 6.38 (s, 1H), 5.80 (s, 1H), 5.68 (s, 1H), 5.61-5.53 (m, 1H), 5.34 (ddd, J=15.3, 7.6, 1.8 Hz, 1H), 5.25 (dd, J=15.0, 9.2 Hz, 1H), 4.61 (dd, J=9.6, 1.9 Hz, 1H), 3.84 (t, J=8.8 Hz, 2H), 3.65-3.60 (m, 1H), 3.59 (s, 3H), 3.62-3.55 (m, 1H), 3.49 (td, J=10.5, 4.5 Hz, 1H), 3.43 (dd, J=11.2, 7.7 Hz, 1H), 3.31 (q, J=8.9 Hz, 1H), 3.28-3.25 (m, 0H), 3.26 (s, 1H), 3.25 (s, 3H), 3.11 (t, J=8.9 Hz, 1H), 2.74 (s, 1H), 2.19-2.06 (m, 3H), 1.99 (s, 5H), 1.84 (s, 3H), 1.74-1.68 (m, 1H), 1.68-1.58 (m, 4H), 1.51 (s, 1H), 1.32 (d, J=6.1 Hz, 3H), 1.26 (s, 3H), 1.20 (q, J=11.4 Hz, 1H), 1.07 (s, 5H), 0.93-0.82 (m, 16H).

### Example 19: 3′-(3-(3-(but-3-yn-1-yl)-3H-diazirin-3-yl)propanoate) Concanamycins A-C (Compounds 28-30)

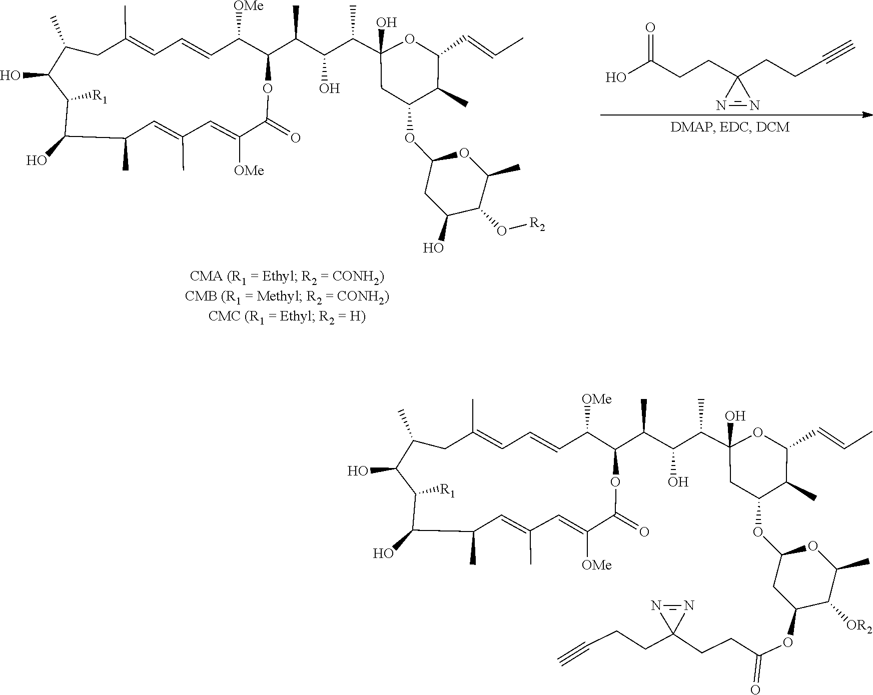

3-(3-(but-3-yn-1-yl)-3H-diazirin-3-yl)propanoic acid (32.6 mg, 196 μmol) was added to a solution of a mixture of concanamycins A-C (75.0 mg, 85.2 μmol) in DCM (5.5 mL) under anhydrous conditions. DMAP (21.9 mg, 179 μmol) and EDC (38.7 mg, 187 μmol) were added to the solution and stirred overnight. The reaction mixture was concentrated and purified by pTLC (8% isopropanol in chloroform). This was done twice to fully assure that each analog was fully separated to yield the desired products. 3′-(3-(3-(but-3-yn-1-yl)-3H-diazirin-3-yl)propanoate) Concanamycin A (Compound 28) ¹H NMR (599 MHz, CDCl₃, 278 K) δ 6.57 (dd, J=15.1, 10.6 Hz, 1H), 6.40 (s, 1H), 5.87 (s, 1H), 5.79 (t, J=10.0 Hz, 1H), 5.68 (d, J=9.5 Hz, 1H), 5.55 (dq, J=13.3, 6.6 Hz, 1H), 5.28 (dd, J=15.3, 8.0 Hz, 1H), 5.22 (dd, J=15.2, 8.9 Hz, 1H), 5.01 (d, J=9.1 Hz, 1H), 4.96 (ddd, J=11.9, 9.4, 5.3 Hz, 1H), 4.70 (d, J=4.3 Hz, 1H), 4.61 (dd, J=9.7, 1.9 Hz, 1H), 4.58 (t, J=9.5 Hz, 1H), 4.15 (d, J=8.1 Hz, 1H), 4.05-4.00 (m, 1H), 3.97 (t, J=9.2 Hz, 1H), 3.87 (d, J=9.1 Hz, 1H), 3.84-3.80 (m, 1H), 3.77 (td, J=10.7, 4.7 Hz, 1H), 3.56 (s, 3H), 3.49-3.44 (m, 1H), 3.41 (dt, J=9.6, 6.1 Hz, 1H), 3.26 (s, 3H), 3.22 (d, J=10.4 Hz, 1H), 2.75-2.69 (m, 1H), 2.32 (dt, J=11.6, 5.8 Hz, 2H), 2.22 (ddd, J=12.3, 5.3, 1.8 Hz, 1H), 2.20-2.15 (m, 1H), 2.13 (t, J=7.5 Hz, 2H), 2.02 (q, J=2.9 Hz, 3H), 2.01 (d, J=7.4 Hz, 3H), 1.97 (s, 3H), 1.94 (d, J=17.2 Hz, 2H), 1.87 (s, 3H), 1.85-1.77 (m, 2H), 1.78-1.71 (m, 2H), 1.69 (d, J=10.1 Hz, 1H), 1.65 (t, J=7.4 Hz, 2H), 1.58 (d, J=6.5 Hz, 3H), 1.53-1.48 (m, 1H), 1.40-1.26 (m, 2H), 1.26 (s, 1H), 1.24 (d, J=6.3 Hz, 5H), 1.19-1.05 (m, 8H), 1.04 (d, J=7.0 Hz, 3H), 0.87 (dd, J=14.7, 6.7 Hz, 6H), 0.82 (d, J=6.9 Hz, 3H). ¹³C NMR (151 MHz, CDCl₃, 278 K) δ 171.76, 166.63, 155.75, 142.15, 141.79, 139.67, 133.31, 132.18, 130.80, 130.69, 127.85, 127.07, 122.96, 99.54, 95.88, 82.62, 81.30, 79.65, 76.09, 75.53, 75.47, 74.91, 74.25, 71.33, 70.09, 69.81, 69.34, 59.08, 55.75, 49.11, 44.74, 43.34, 41.32, 41.19, 39.74, 36.89, 36.85, 36.31, 34.58, 33.89, 31.95, 29.70, 28.55, 27.74, 27.59, 25.52, 24.92, 22.75, 21.69, 17.80, 17.40, 16.80, 16.39, 14.17, 13.38, 13.23, 11.67, 9.32, 7.12. 3′-(3-(3-(but-3-yn-1-yl)-3H-diazirin-3-yl)propanoate) Concanamycin B (Compound 29)

¹H NMR (599 MHz, CDCl₃, 323 K) δ 6.53 (dd, J=15.1, 10.6 Hz, 1H), 6.37 (s, 1H), 5.80 (d, J=10.6 Hz, 1H), 5.65 (s, 1H), 5.55 (dq, J=13.1, 6.4 Hz, 1H), 5.34-5.27 (m, 1H), 5.23 (dd, J=15.2, 8.9 Hz, 1H), 5.03 (d, J=9.1 Hz, 1H), 4.96 (ddd, J=12.1, 9.3, 5.2 Hz, 1H), 4.63-4.53 (m, 4H), 4.07-4.01 (m, 1H), 3.97 (dd, J=10.3, 7.6 Hz, 1H), 3.85 (q, J=9.4 Hz, 2H), 3.77 (td, J=10.7, 4.6 Hz, 1H), 3.57 (s, 2H), 3.40 (dq, J=12.4, 6.3 Hz, 1H), 3.28 (s, 1H), 3.25 (s, 3H), 2.76-2.69 (m, 1H), 2.30 (dd, J=12.0, 4.8 Hz, 1H), 2.20 (dt, J=15.4, 7.3 Hz, 2H), 2.12 (t, J=7.6 Hz, 2H), 2.00 (dt, J=17.1, 4.8 Hz, 8H), 1.82 (d, J=10.8 Hz, 3H), 1.82-1.73 (m, 2H), 1.76-1.71 (m, 1H), 1.73-1.65 (m, 1H), 1.63 (t, J=7.4 Hz, 2H), 1.59 (d, J=6.5 Hz, 3H), 1.38-1.16 (m, 5H), 1.09 (s, 0H), 1.06 (t, J=7.1 Hz, 7H), 0.88 (t, J=7.9 Hz, 6H), 0.82 (d, J=6.9 Hz, 3H). 3′-(3-(3-(but-3-yn-1-yl)-3H-diazirin-3-yl)propanoate) Concanamycin C (Compound 30). ¹H NMR (599 MHz, CDCl₃) δ 6.58-6.51 (m, 1H), 6.38 (s, 1H), 5.76 (s, 1H), 5.68 (s, 1H), 5.56 (tt, J=13.0, 7.2 Hz, 1H), 5.33-5.26 (m, 1H), 5.22 (dd, J=15.4, 9.2 Hz, 1H), 5.02 (d, J=8.9 Hz, 1H), 4.81 (ddd, J=12.7, 8.1, 5.2 Hz, 1H), 4.64-4.59 (m, 2H), 4.03 (s, 2H), 4.01-3.95 (m, 1H), 3.91-3.83 (m, 1H), 3.82 (s, 2H), 3.78 (td, J=10.7, 4.8 Hz, 1H), 3.56 (d, J=1.1 Hz, 3H), 3.50-3.45 (m, 1H), 3.31 (ddt, J=11.0, 8.2, 3.9 Hz, 2H), 3.29-3.24 (m, 3H), 2.73 (s, 1H), 2.63-2.46 (m, 4H), 2.35-2.29 (m, 1H), 2.31 (s, 2H), 2.24-2.15 (m, 2H), 2.00 (td, J=2.5, 1.0 Hz, 1H), 1.98 (s, 5H), 1.96-1.91 (m, 3H), 1.86 (s, 2H), 1.78-1.59 (m, 5H), 1.40-1.32 (m, 3H), 1.34-1.30 (m, 3H), 1.25 (h, J=6.5, 5.9 Hz, 3H), 1.18 (dt, J=12.1, 4.8 Hz, 1H), 1.17-1.09 (m, 2H), 1.10-1.01 (m, 7H), 0.96-0.84 (m, 3H), 0.86 (s, 5H), 0.82 (d, J=6.9 Hz, 2H).

### Example 20: 3′-pent-4-ynoate Concanamycins A-C (Compounds 31-33)

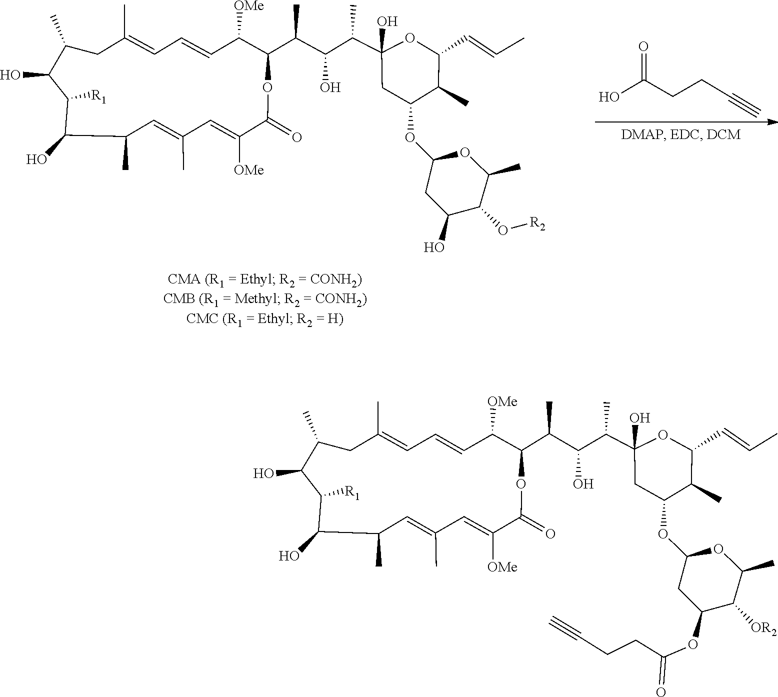

Pent-4-ynoic acid (11.3 mg, 116 μmol) was added to a solution of a mixture of concanamycins A-C (50.0 mg) in DCM (6.7 mL) under anhydrous conditions. DMAP (14.8 mg, 121 μmol) and EDC (26.2 mg, 127 μmol) were added to the solution and stirred overnight. The reaction mixture was concentrated and purified by pTLC (8% isopropanol in chloroform). This was done twice to fully assure that each analog was fully separated to yield the desired products. 3′-pent-4-ynoate Concanamycins A-C (Compounds 31-33) ¹H NMR (599 MHz, CDCl₃) δ 6.55 (dd, J=15.1, 10.7 Hz, 1H), 6.39 (s, 1H), 5.78 (s, 2H), 5.68 (s, 1H), 5.59-5.50 (m, 1H), 5.30 (s, 1H), 5.33-5.26 (m, 0H), 5.22 (dd, J=14.6, 7.5 Hz, 1H), 5.02 (s, 1H), 4.64-4.50 (m, 2H), 4.26-4.17 (m, 1H), 4.03 (d, J=12.7 Hz, 2H), 3.97 (dd, J=10.2, 7.8 Hz, 1H), 3.86 (t, J=9.1 Hz, 1H), 3.77 (td, J=10.8, 4.7 Hz, 1H), 3.56 (s, 3H), 3.48 (dtd, J=10.7, 7.3, 4.1 Hz, 1H), 3.44-3.34 (m, 1H), 3.26 (s, 3H), 3.12 (s, 1H), 2.73 (s, 1H), 2.67-2.57 (m, 1H), 2.59-2.50 (m, 2H), 2.48 (dt, J=9.0, 4.5 Hz, 1H), 2.36 (s, 0H), 2.31 (dd, J=12.0, 4.8 Hz, 1H), 2.21 (tdd, J=13.5, 6.0, 3.0 Hz, 2H), 2.01 (q, J=2.0, 1.5 Hz, 0H), 1.98 (s, 6H), 1.97-1.91 (m, 2H), 1.79-1.63 (m, 5H), 1.61 (s, 8H), 1.60-1.56 (m, 3H), 1.51 (dd, J=14.6, 2.0 Hz, 1H), 1.51 (s, 1H), 1.40-1.32 (m, 2H), 1.32 (s, 4H), 1.30-1.22 (m, 7H), 1.20 (d, J=6.2 Hz, 4H), 1.20-1.13 (m, 1H), 1.14-1.09 (m, 1H), 1.10-1.03 (m, 7H), 0.97-0.87 (m, 3H), 0.86 (s, 5H), 0.82 (d, J=6.9 Hz, 3H), -0.60 (s, 2H).

### Example 21: 3′-pentanoate Concanamycin A (Compound 34)

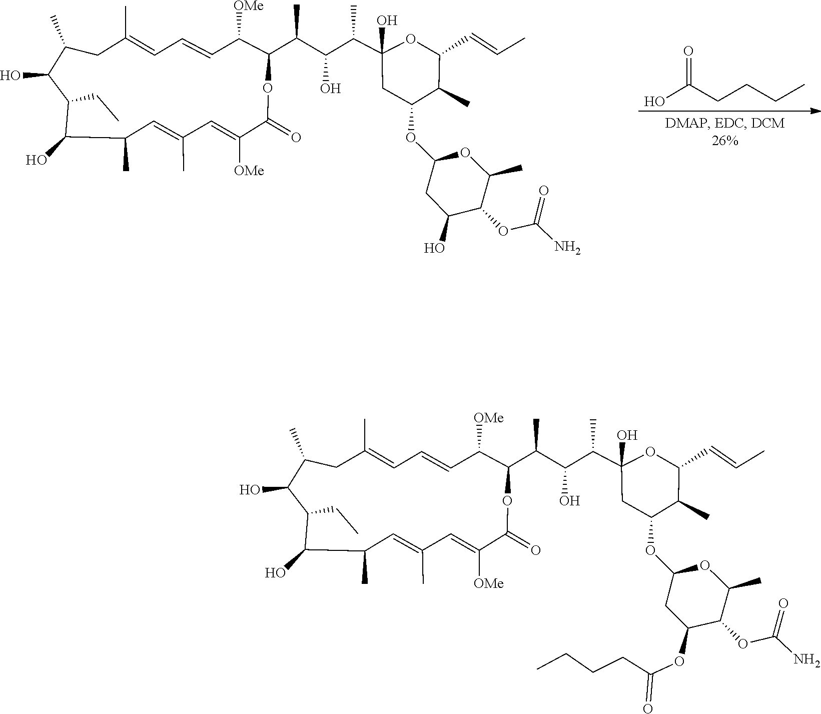

DMAP (7.4 mg, 61 μmol, 2.1 eq) and EDC (12.2 mg, 63.5 μmol, 2.2 eq) were added to a solution of CMA (25.0 mg, 28.9 μmol) in DCM (4.0 mL) under anhydrous conditions and stirred. Pentanoic acid (6.3 μL, 57.7 μmol, 2 eq) was then added in aliquots (1/2, followed by 2nd half 15 minutes later) to help discourage double addition products. The reaction was closely monitored by TLC (8% isopropanol/CHCl₃). After 2.5 hours the reaction mixture was concentrated and immediately purified by pTLC (4% isopropanol/CHCl₃) to yield 7 mg (26%) of white product that was highly soluble in CHCl₃. 3′-pentanoate Concanamycin A (Compound 34) ¹H NMR (599 MHz, CDCl₃) δ 6.56 (dd, J=15.1, 10.7 Hz, 1H), 6.40 (s, 1H), 5.87 (s, 1H), 5.81-5.75 (m, 1H), 5.68 (d, J=9.8 Hz, 1H), 5.55 (dq, J=13.1, 6.6 Hz, 1H), 5.28 (dd, J=14.9, 7.9 Hz, 1H), 5.22 (dd, J=15.0, 9.1 Hz, 1H), 5.01 (dd, J=9.0, 6.2 Hz, 1H), 5.00 - 4.91 (m, 1H), 4.73-4.66 (m, 1H), 4.64-4.57 (m, 2H), 4.02 (d, J=10.3 Hz, 1H), 3.97 (t, J=9.2 Hz, 1H), 3.90-3.81 (m, 2H), 3.77 (td, J=10.8, 4.7 Hz, 1H), 3.58 (d, J=10.9 Hz, 1H), 3.56 (s, 3H), 3.44-3.36 (m, 1H), 3.26 (s, 3H), 3.22 (d, J=10.3 Hz, 1H), 2.73 (s, 1H), 2.31 (dt, J=10.7, 7.5 Hz, 4H), 2.20 (tdd, J=14.8, 7.4, 4.1 Hz, 2H), 2.03 (s, 1H), 1.97 (s, 3H), 1.96-1.94 (m, 0H), 1.87 (s, 2H), 1.76 (d, J=7.4 Hz, 2H), 1.75-1.63 (m, 2H), 1.63-1.53 (m, 5H), 1.32 (p, J=7.5 Hz, 2H), 1.25 (dd, J=11.1, 6.2 Hz, 5H), 1.15 (dd, J=15.7, 8.5 Hz, 1H), 1.07 (td, J=14.0, 12.9, 7.2 Hz, 7H), 0.89 (td, J=7.5, 2.8 Hz, 9H), 0.84 (dd, J=21.0, 7.1 Hz, 4H).

### Example 22: 19-deoxy-21-pent-4-ynoate Bafilomycin (Compound 34)

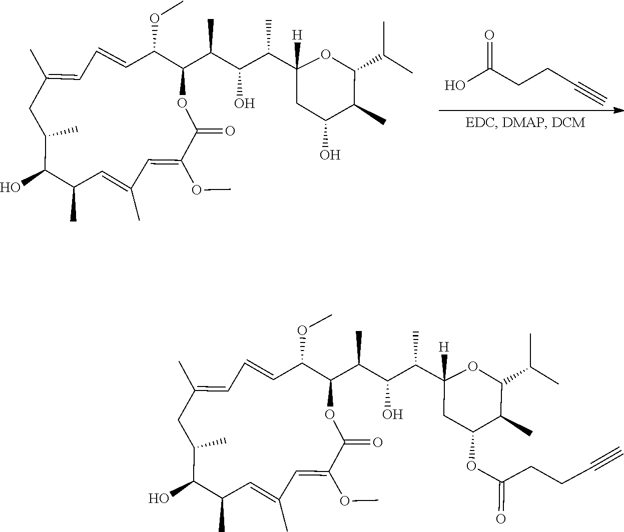

Pent-4-ynoic acid(4.85 mg, 49.4 μmol, 3 eq) was added to a solution of bafilomycin A₁ (10.0 mg, 16.5 μmol) in DCM (2.0 mL) at RT under anhyrdrous conditions. DMAP(6.24 mg, 51.1 μmol, 3.1 eq) and EDC (10.4 mg, 54.4 μmol, 3.3 eq) were added and the reaction was left to stir overnight. The reaction mixture was then concentrated and immediately purified by pTLC (15% acetone/hexanes) to yield 19-deoxy-21-pent-4-ynoate bafilomycin. 19-deoxy-21-pent-4-ynoate Bafilomycin (Compound 34) ¹H NMR (599 MHz, Acetone) δ 6.71-6.60 (m, 2H), 5.93 (d, J=10.8 Hz, 1H), 5.82 (dt, J=9.2, 1.2 Hz, 1H), 5.29 (dd, J=15.1, 8.4 Hz, 1H), 5.24-5.13 (m, 1H), 4.81 (dd, J=5.8, 2.4 Hz, 1H), 4.66 (td, J=10.7, 4.8 Hz, 1H), 4.11-4.00 (m, 1H), 3.80 (dtd, J=10.5, 4.4, 2.3 Hz, 1H), 3.69 (dd, J=21.1, 4.6 Hz, 1H), 3.63 (d, J=9.3 Hz, 3H), 3.46 (ddd, J=11.6, 8.2, 1.9 Hz, 1H), 3.23 (d, J=8.5 Hz, 3H), 2.95 (dd, J=10.0, 2.2 Hz, 1H), 2.78 (ddd, J=9.3, 7.0, 2.4 Hz, 1H), 2.71 (t, J=7.1 Hz, 2H), 2.61-2.51 (m, 4H), 2.48 (tdd, J=7.2, 2.7, 1.2 Hz, 2H), 2.39 (dt, J=10.7, 2.6 Hz, 2H), 2.16-2.06 (m, 2H), 2.03 (s, 0H), 1.98 (dd, J=9.0, 1.2 Hz, 3H), 1.90 (s, 1H), 1.91-1.85 (m, 3H), 1.83 (dd, J=14.7, 11.0 Hz, 1H), 1.69 (ddd, J=12.3, 7.0, 3.6 Hz, 1H), 1.57 (tq, J=10.1, 6.5 Hz, 1H), 1.26-1.15 (m, 1H), 1.05 (t, J=6.4 Hz, 3H), 0.98 (dd, J=6.9, 3.5 Hz, 3H), 0.94 (d, J=7.1 Hz, 3H), 0.90-0.79 (m, 12H). ¹³C NMR (151 MHz, Acetone) δ 172.72, 171.84, 166.38, 143.10, 142.46, 141.32, 133.73, 133.17, 132.35, 128.37, 126.08, 84.85, 84.04, 83.65, 83.53, 82.43, 77.87, 77.34, 77.32, 70.66, 70.50, 70.47, 60.04, 55.90, 42.25, 40.85, 39.01, 38.90, 38.68, 37.30, 36.78, 34.33, 34.28, 29.22, 22.79, 21.41, 20.23, 17.59, 14.97, 14.91, 14.72, 14.13, 12.62, 10.81, 8.79.

### Example 23: Cell Based Assay

A high throughput cell-based assay has been developed to screen mixtures of compounds produced by microbial cultures [natural product extracts (NPEs)]. To screen small molecules and NPE libraries for Nef inhibitors, a “mix and measure” high throughput flow cytometric assay was used to assess Nef-dependent MHC-I downmodulation. A CEM T cell line was used that expresses MHC-I HLA-A2 (CEM-A2) and behaves like primary T cells with respect to Nef-dependent MHC-I downmodulation (Roeth et al., 2004; Wonderlich et al., 2011). The T cells were transiently transduced with an adenoviral vector that expresses Nef under the control of an elongation factor 1 alpha promoter (Kasper et al., 2005). Two days after transduction, the Nef-expressing T cells were added to compounds in 384 well plates and harvested the next day. Over 26,000 NPEs were screened using this strategy and employed a number of counter screens that allowed compounds causing non-specific increases in cellular fluorescence to be eliminated.

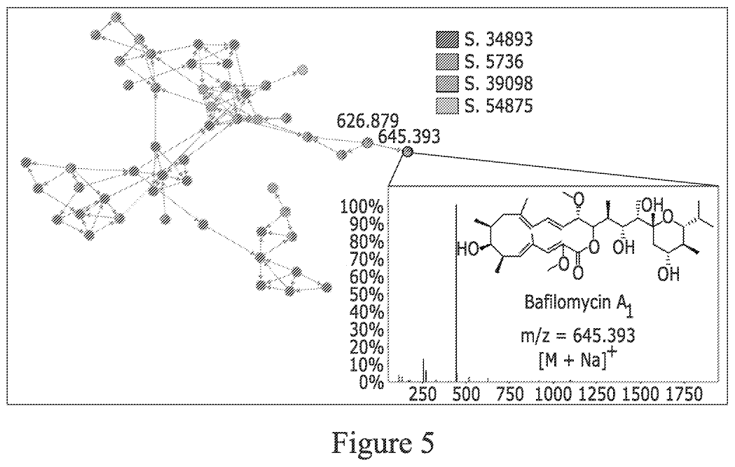

Testing then focused on a subset of ten compounds that reversed MHC-I expression in HIV-1 infected T cells. Positive hits were confirmed by re-preparing the NPE from 100 ml cultures. After 14-18 days of growth, the cultures were harvested by centrifugation. The resulting cell free broth was subjected to solid phase extraction using 15 g/L of Amberlite XAD-16. An LC-MS/MS generated molecular network (Wang et al., 2016) analysis of NPEs from several high-priority Nef inhibitory strains identified a common m/z of 645.393 corresponding to the presence of a plecomacrolide family member [bafilomycin (Baf) A1]. Extracts from Streptomyces sp. 54875 lacked this characteristic ion suggesting activity associated with this strain is unique (FIG. 5), however, work on this strain is outside the scope of this proposal.

The previously identified molecular target of plecomacrolide natural products is vacuolar ATPase (V-ATPase) and these compounds are well known inhibitors of V-ATPase-dependent lysosomal acidification (Drose and Altendorf, 1997). However, lysosomal inhibition by these compounds is dispensable for Nef inhibition (FIG. 3).

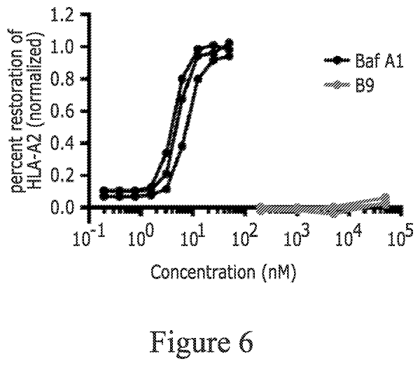

Based on the identification of a subset of Nef inhibitors as Baf family members, several commercially available plecomacrolide compounds were obtained, including Baf A1, Baf B1, Baf C1, Baf D and CMA. Remarkably, a six-log range of potencies was observed from the five molecules (FIG. 2). These natural products were far more potent than a structurally unrelated commercially available synthetic Nef inhibitor (B9) reported to enhance CD8 T cell-mediated cytokine secretion and elimination of latently HIV-1 infected cells (FIG. 6) (Mujib et al., 2017)

### Example 24

Determination of anti-Nef activity: To identify molecules with enhanced Nef inhibition, decreased toxicity and decreased inhibition of lysosomes, the analogs of the disclosure are screened initially for Nef inhibitory activity using a high throughput assay in HIV-1 infected CEM-A2 cells. These cells are infected with an HIV-1 reporter construct for 48 hours and then exposed to potential inhibitors for 24 hours. Cells are harvested and stained for flow cytometric analysis of HLA-A2 surface expression, and the fold downmodulation of HLA-A2 are calculated by comparing expression on uninfected cells to that on infected cells. Analogs that inhibit Nef with comparable potency relative to the unmodified compound have their inhibitory activity validated in human PBMCs.

The EC50 for Nef inhibition in primary cells is determined for each analog that was active in CEM-A2 cells. PBMCs is isolated from leukopaks, CD8-depleted, and stimulated with PHA. Stimulated PBMCs cultured in the presence of IL-2 is infected with HIV-1 and exposed to compounds as for the CEM-A2 cells, and the level of Nef inhibition is calculated for each analog in the same way. A titration of each analog is performed to accurately determine the EC50 and identify modifications that enhance the Nef inhibitory activity relative to the best earlier generation compounds, along with Baf A1 and CMA.

Determination of toxicity: For analogs found to have comparable or enhanced Nef inhibitory activity relative to their respective previous generation compounds, their toxicity is further characterized in PHA-activated primary human PBMCs. Cells are exposed to a titration of each analog for 72 hours in culture, at which point viability is assessed relative to a solvent control by MTT assay. The MTT assay accounts for defects in proliferation, survival, and metabolic capacity, providing a broad assessment of cytotoxicity. Changes in toxicity inform further compound optimization, and analogs with increased separation between toxic concentrations and Nef inhibitory concentrations is studied further.

Determine effect on V-ATPase activity: Plecomacrolides are known inhibitors of V-ATPase that neutralize normally acidified lysosomes. Preliminary data demonstrate that plecomacrolides have varying potencies for lysosome neutralization, and that this activity is separable from Nef inhibition (FIG. 3). Thus, analogs of the disclosure are further characterized by measuring their effects on lysosome acidification. PBMCs are treated with titrations of each analog at a density of 1 million cells/mL for 24 hours. Cells are then incubated in PBS with 100 nM LysoTracker® Red DND-99 (Fisher L7528) at a density of 1 million cells/mL for 1 hour at 37° C., at which point they are washed twice and fixed in 2% PFA for 20 minutes at room temperature and analyzed by flow cytometry as shown in (FIG. 3).

The results of the anti-Nef activity, toxicity, and lysosomal acidification assays are summarized in Table B, below.

TABLE B

| Compound | Nef IC50 | Lyso IC50 | Viability |
|---|---|---|---|
| CMA | 0.09849 | 0.9774 | 0.455 |
| CMC | 0.7799 | 2.716 | 1.292 |
| Virustomycin | 0.6878 | 3.677 | 3.849 |
| BafA1 | 11.51 | 45.18 | 14.78 |
| BafA2 | 11.57 | 25.34 | 13.24 |
| 19-Deoxy Baf A1 | 33.94 | 111.3 | 48.5 |
| 19-Deoxy-21-Methoxy Baf A1 | 20.1 | 49.56 | 29.21 |
| 19-Deoxy-20-ene Baf A1 | 146.8 | 340.8 | 234 |
| 19-Deoxy-21-allylester Baf A1 | 56 | 152.4 | 79.95 |
| Baf A1 Ring Open | 308.6 | 1000 | 1000 |
| PC-766B | 1.133 | 2.724 | 1.315 |
| Leucanicidin | 1.159 | 2.043 | 1.393 |
| CMB | 0.9755 | 29.64 | 7.091 |
| 21 Dual Linear Bafilomycin Probe | 12.01 | 28.28 | 15.53 |
| 21 Alkyne Bafilomycin Probe | 8.692 | 29.1 | 13.76 |
| 21-X Bafilomycin Probe | 1000 | 1000 | 1000 |
| 21-Y Bafilomycin Probe | 1000 | 1000 | 1000 |
| 21-Y Bafilomycin Probe | 1000 | 1000 | 1000 |
| 7 Alkyne 21-Y Bafilomycin Probe | 1000 | 1000 | 1000 |
| 19-Deoxy-21(4-Pentynoate) Baf A1 | 21.27 | 76.4 | 36.01 |
| CMF | 1.522 | 6.268 | 2.824 |
| 21-Deoxy CMF | 4.286 | 23.31 | 8.98 |
| 2-hydroxy-21-deoxy CMF | 358.4 | 1000 | 510.4 |
| 21-Deoxy CMC | 1.22 | 7.036 | 1.665 |
| 21-Acetyl Baf | 4.625 | 22.68 | 8.891 |
| Dual Linear Probe CMA | 0.693 | 4.869 | 2.427 |
| 23-Dual Linear Probe CMB | 1.725 | 23.2 | 8.848 |
| 23-Dual Linear Probe CMC | 1.481 | 4.698 | 2.109 |
| 23-Alkyne CMA | 0.179 | 1.096 | 1.23 |
| 23-Alkyne CMB | 0.8334 | 5.754 | 6.324 |
| 23-Alkyne CMC | 1.498 | 5.725 | 1.753 |
| CMF | 1.522 | 6.268 | 2.824 |
| 21-Deoxy CMF | 4.286 | 23.31 | 8.98 |
| 2-Hydroxy-21-Deoxy CMF | 358.4 | 1000 | 510.4 |
| 21-Deoxy CMC | 1.22 | 7.036 | 1.665 |
| 21-Pentanoate Baf A1 | 37.1 | 62.3 | 60.97 |
| 21-Nonanoate Baf A1 | 128.2 | 145.6 | 138.5 |
| 21-Penta-3-enoate Baf A1 | 24.11 | 60.25 | 46.76 |
| 21-penta-2,4-dienoate Baf A1 | 16.16 | 25.12 | 24.12 |
| 21-benzoate Baf A1 | 91.59 | 139.2 | 129.7 |
| 7-One Baf A1 | 1000 | 1000 | 1000 |
| 7,21-Dione Baf A1 | 1000 | 1000 | 1000 |

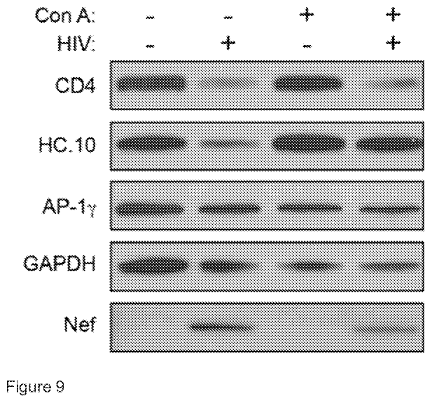

Assess capacity to enhance CTL recognition: Because Nef-dependent MHC-I downmodulation limits anti-HIV-1 CTL recognition, effective Nef inhibitors such as CMA enhance anti-HIV-1 CTL killing (FIGS. 1 and 8). Therefore, the effect of promising Nef inhibitor analogs is generated as disclosed herein on anti-HIV-1 CTL killing using the in vitro flow cytometric CTL killing assay developed previously (FIGS. 1 and 8 [symbol] (Collins et al., 1998). These studies utilize CTL clones reactive against various HIV-1 epitopes known to be affected by Nef (Collins et al., 1998).

Determine effect of inhibitors on other Nef allotypes: Because HIV sequences vary, the extent to which each analog is active against Nef proteins from different HIV isolates is assessed. In previous studies, a panel of eleven Nef proteins from different clades has been described. The capacity to potently downmodulate MHC-I, CD4 and CD8β were conserved activities of Nef across HIV-1 clades (Leonard et al., 2011). Moreover, similar results were obtained in a collaborative study examining a total of 42 nef alleles from 16 different primate lentiviruses. These alleles represent most major lineages of primate lentiviruses, as well as nonpandemic HIV-1 strains and the direct precursors of HIV-1 (SIVcpz and SIVgor) (Heigele et al., 2012). Thus, analogs disclosed herein are analyzed to determine if they are active broadly against a variety of HIV-1 clades and SIV. By testing efficacy against SIV Nef, it can be determined the likelihood that the SIV macaque model would be a viable way to test inhibitor efficacy.

In vitro pharmaceutical profiling. The PK Core at UM conducts stability testing in vitro using liver microsomes from mouse and human liver cells (Tong et al., 2006). For these experiments, a select set of 5 to 10 top priority compounds are incubated with liver microsomes and the amount of the compound is measured over time using LC-MS/MS spectra to determine the half-life. In addition, the PK core identifies the metabolites generated during the liver microsome incubation using LC-MS/MS with various scan modes and by using the fragmentation pathway of the compound in MS/MS spectra. This allows the specific identification of unstable moieties that are targeted iteratively for synthetic optimization (Trunzer et al., 2009).

In vivo PK studies: In vivo PK studies are performed in mice with both oral and IV administration routes (Zhang et al., 2013a). The concentration of a select set of 5 to 10 priority compounds in plasma isquantified using LC-MS/MS as well as by direct bioassay using reversal of MHC-I downmodulation in HIV-1 infected cells as a readout. PK parameters are calculated with non-compartmental analysis using the PK software package, WinNonlin, to obtain absorption and elimination rate constants, half-life, volume of distribution, clearance, maximal drug concentration, oral bioavailability and area under the concentration versus time curve. The lead compounds are selected based on favorable PK profiles. The dose regimen for efficacy studies is optimized based on PK parameters and minimal effective concentration. Finally, interspecies scaling of PK and dose regimen is performed (Zou et al., 2012).

### Example 24

CMA is the least toxic and most efficacious plecomacrolide with a relatively wide (>five-fold) therapeutic window in HIV-1 infected primary T cells: To determine the EC50 for Nef inhibition for a number of Baf analogs and CMA, peripheral blood mononuclear cells (PBMCs) were from leukopaks, CD8-depleted, and stimulated with PHA. Stimulated PBMCs cultured in the presence of IL-2 were infected with HIV-1. After 48 hours, they were exposed to compounds for 24 hours and MHC-I HLA-A2 surface levels were measured using the HLA-A2 selective monoclonal antibody BB7.2 (Parham and Brodsky, 1981). A range of potencies over a six-log range of plecomacrolide concentrations with CMA was observed being the most potent and Baf D being the least potent (FIG. 2). To assess their toxicity, an MTT assay was used, which measures 4-succinate dehydrogenase activity. Metabolically active cells reduce MTT to MTT-formazan leading to a color change. For this analysis, PHA-activated primary human PBMCs were exposed to a range of concentrations of each drug continuously for 72 hours of culture. Results from this analysis superimposed onto the EC50 results show a narrow window between effective and toxic doses for Baf A1 and Baf C1. However, this window expands to a five-fold difference between effective and toxic doses for CMA (FIG. 4).

### Example 25

Inhibitory effects of CMA on lysosome acidification are separable from Nef inhibition in HIV-1 infected primary T cells: Plecomacrolides are known inhibitors of V-ATPase (Drose and Altendorf, 1997) and this activity results in neutralization of normally acidified lysosomes. To determine whether the low dose of CMA needed for effective inhibition of Nef was sufficient for V-ATPase activity, the lysosomal function was assesed using LysoTracker® staining of acidified lysosomes. LysoTracker® is a fluorophore linked to a weak base that is partially protonated at neutral pH. This form of the probe is capable of freely permeating cell membranes of live cells. In acidic compartments, protonation creates a form that was impermeable causing the probe to accumulate inside the lysosome. To assess lysosome activity in the presence of concentrations of CMA that inhibit Nef, PHA-stimulated PBMCs were treated with varying concentrations of drug for 24 hours exactly as described for assessment of Nef inhibition. They were then incubated in PBS with 100 nM LysoTracker® Red DND-99 (Fisher L7528) at a density of 1 million cells/mL for 1 hour at 37° C., at which point they were washed twice and fixed in 2% PFA for 20 minutes at room temperature before flow cytometric analysis. Results from this analysis overlaid onto a curve of Nef inhibitory activity revealed that substantially more CMA was needed to inhibit lysosome acidification than was needed to inhibit Nef (>ten-fold, FIG. 3). These results were confirmed by demonstrating that doses of CMA sufficient to reverse Nef-dependent degradation on MHC-I proteins did not alter Nef-dependent lysosomal degradation of CD4 in HIV-1 infected primary T cells sorted for expression of a marker protein (PLAP). Based on these compelling results, it was concluded that the mechanism through which low dose CMA reverses MHC-I downmodulation was distinct from lysosomal inhibition. Thus, the next step was to generate plecomacrolide analogs with significantly reduced capacity for lysosomal degradation and reduced toxicity, while retaining and increasing their ability to inhibit Nef.

As a first step to understand how CMA and Bafs function to inhibit Nef activity, it was considered that they might simply act by reducing Nef transcription. While this mechanism of action would be efficacious at inhibiting Nef, HIV LTR suppression would counteract current strategies for reservoir eradication, which rely upon reversal of latency and activation of HIV gene transcription. To avoid this problem, the compounds that were selected for further study inhibited Nef regardless of the promoter used to drive Nef expression—each inhibitor disrupted Nef-mediated MHC-I downmodulation regardless of whether Nef was expressed from an adenoviral-vector system under the control of the elongation factor 1 alpha promoter or within its normal HIV-1 context under the control of the HIV-1 5′LTR. The use of a reporter that enabled transcriptional monitoring via expression of PLAP cloned into the env open reading frame (Chen et al., 1996) confirmed the conclusion. The inhibitors did not reduce HIV-1 LTR activity based on PLAP expression (FIG. 1). Moreover, these compounds did not significantly alter the amount of Nef protein in HIV-1 infected primary T cells based on western blot analysis (e.g. FIG. 9).

Nef uses functionally distinct and separable domains to downmodulate MHC-I and CD4. To better understand the selectivity of the inhibitors, it was asked whether each one disrupts the effect of Nef on both molecules or just MHC-I. Because Vpu and Nef downmodulate CD4, this question was answered using T cells transduced with an adenoviral vector expressing Nef alone or with HIV-1 constructs lacking Vpu. The effects of these inhibitors were found to be selective when used at concentrations that do not disrupt lysosomal inhibition. For example, as shown in FIG. 9, MHC-I, as detected by monoclonal antibody HC10, and CD4 are degraded in Nef-expressing primary T cells. Addition of CMA at concentrations sufficient to reverse Nef-mediated degradation of MHC-I did not significantly alter Nef-mediated CD4 degradation (FIG. 9). Because Nef affected MHC-I downmodulation and CD4 downmodulation using different pathways this information was useful in narrowing down potential drug targets. Additionally, these results were consistent with data that at concentrations used in assays, CMA was not acting by lysosomal inhibition. Inhibition of lysosomes would rescue both MHC-I and CD4 protein levels in Nef expressing cells. In contrast, efficient lysosomal degradation of CD4 was observed but not MHC-I (FIG. 9).

### Example 26

Determine the effect of CMA on the intracellular localization of Nef, MHC-I, AP-1, beta-COP and V-ATPase subunits in Nef expressing or control T cells: The HIV-1 Nef protein is ˜25 kDa cytoplasmic protein containing a myristoyl group at its N-terminus that allows it to associate with the inner leaflet of the plasma membrane. Confocal fluorescent microscopy studies indicated that Nef accumulates at the juxtanuclear region of the cell co-localizing with AP-1 (Janvier et al., 2001) and MHC-I (Williams et al., 2002). Normally, MHC-I is observed as a ring around the outer membrane of cells but in Nef expressing T cells, MHC-I staining was diminished and co-localized with AP-1 in the region of the trans-Golgi network (TGN) (Roeth et al., 2004; Schaefer et al., 2008). Additionally, MHC-I was in late endosomes and multi-vesicular bodies co-localizing with CD4 in Nef expressing T cells (Roeth et al., 2004; Schaefer et al., 2008). Moreover, inhibition of lysosomal degradation revealed co-localization of MHC-I with markers of lysosomes (Lamp1) in Nef expressing T cells (Roeth et al., 2004; Schaefer et al., 2008).

To elucidate the mechanism through which pM concentrations of CMA inhibit Nef without disrupting lysosomal degradation, HIV-1 infected primary T cells plus or minus Nef expression is treated with CMA overnight. The cells are stained with antibodies directed at MHC-I, AP-1, Nef, beta-COP and subunits of V-ATPase to determine whether co-localization of these proteins with one another or with organelle markers is altered in the presence of low dose CMA (0.5 nM). It is expected that low dose CMA treatment results in normalization of MHC-1 localization to the plasma membrane. If CMA disrupts formation of Nef-dependent complexes, it is expected that reduced co-localization of MHC-I with AP-1, beta-COP and Nef. In addition, it is determined whether Nef, AP-1 and/or MHC-1 co-localize with subunits of the V-ATPase and if so whether low dose CMA disrupts this co-localization. Quantification of colocalization is performed using ImageJ software. Each experiment is repeated a minimum of three times and statistically significant inhibitor-dependent changes is interpreted as an indication that the inhibitor affects that step.

### Example 27

Measure the effect of inhibitors on the rate of MHC-I transport to the cell surface plus or minus Nef: Nef reduces MHC-I expression by re-routing newly synthesized MHC-I from the trans-Golgi (TGN) and preventing its transport to the cell surface. CMA treatment restores MHC-I to the cell surface based on flow cytometric assays that assess steady state surface MHC-I expression. It is unclear however whether CMA-treatment completely normalizes MHC-I transport by disrupting AP-1-dependent sorting to endosomes at the TGN or whether it re-routes MHC-I from post-TGN vesicles to the plasma membrane at a later step. It is expected that the latter possibility would result in slowing of MHC-I transport relative to untreated control cells. To assess this possibility, a surface transport assay is used to measure Nef-dependent MHC-I transport to the cell surface. Briefly, newly synthesized proteins are radiolabeled with ³⁵S amino acids followed by a chase period over six hours in the presence of a cell-impermeable biotinylation reagent (sulfo-NHS-biotin) as previously described (Kasper and Collins, 2003). MHC-I HLA-A2 is immunoprecipitated with monoclonal antibody BB7.2. One-third of the immunoprecipitate is analyzed by SDS-PAGE to assess total HLA-A2 expression. Two-thirds is reprecipitated with avidin beads to isolate cell surface HLA-A2, which is quantified following separation on SDS-PAGE using a phosphorimager. The percentage of recovered surface HLA-A2 relative to total HLA-A2 synthesized by control and Nef-expressing plus or minus CMA is measured over a six-hour time course. Results are measured from three independent experiments and statistically significant increases in transport in CMA-treated samples relative to control untreated cells is an indication that CMA does not fully normalize MHC-I traffic and probably acts at a post-TGN step of Nef-dependent MHC-I trafficking.

### Example 28

Determine whether the inhibitor(s) affect the ability of Nef to stabilize interactions between MHC-I, AP-1 and/or [symbol] COP and ARF-1: It was previously demonstrated that in HIV-1 infected primary T cells, Nef promoted a physical interaction between endogenous AP-1 and MHC-I that can be detected by immunoprecipitating MHC-I complexes from digitonin lysates of HIV-1 infected primary T cells (Roeth et al., 2004). This interaction uses a novel AP-1 binding site that requires amino acids in the MHC-I cytoplasmic tail as well as Nef (Roeth et al., 2004). Based on these studies and work from other groups (Jia et al., 2012; Shen et al., 2015), binding of AP-1 to the Nef— MHC-1 complex is generally accepted as being a crucial step necessary for inhibition of antigen presentation by HIV-1. To determine whether low dose CMA treatment disrupts or prevents the formation of this complex, control or CMA treated HLA-A2+ primary T cells infected with wild type or [symbol] nef HIV-1 is utilized. The anti-HLA-A2 monoclonal antibody BB7.2 crosslinked to protein NG beads is used to immunoprecipitate HLA-A2 from digitonin lysates of control or CMA treated samples. The immunoprecipitates is analyzed by western blot analysis to assess coprecipitation of AP-1 with the complexes. Input controls are included to assess whether CMA treatment affected expression MHC-I, Nef and/or AP-1. Each experiment is repeated a minimum of three times and statistically significant differences are interpreted as an indication that the inhibitor affects that step. Preliminary studies using small mixtures of natural products that include Baf molecules indicate they disrupt or prevent the formation of the Nef-MHC-I-AP-1 complex relative to the amount of co-precipitating MHC-I HLA-A2. Studies with pure Baf and CMA are needed to confirm these results.

ARF-1 is a clathrin regulatory protein that, upon binding GTP, undergoes a conformational change exposing a myristoyl group that inserts into membranes and subsequently stabilizes AP-1 or COP-I coatomer. ARF-1 activity is required for Nef-dependent MHC-I trafficking via AP-1 and ARF-1 can be coprecipitated with the AP-1-MHC-I-Nef complex (Wonderlich et al., 2011). It can also be identified in complexes visualized by via cryo-electron microscopy of Nef-MHC-I-AP-1 complexes (Shen et al., 2015). To determine whether CMA disrupts the formation of this complex, MHC-I HLA-A2 is immunoprecipitated from Nef-expressing T cells transduced with a retroviral construct expressing Myc-tagged ARF-1 as described previously (Wonderlich et al., 2011). A monoclonal antibody directed against Myc is used to detect immunoprecipitation of ARF-1. These studies are performed a minimum of three times and statistically significant differences are interpreted to indicate that CMA directly or indirectly disrupts this interaction.

MHC-I and CD4 are ultimately found in the same Rab7+ vesicles and are both targeted for degradation via the activity of the Nef-interacting protein, beta-COP. Nef contains two separable beta-COP binding sites. One site, an arginine (RXR) motif in the N-terminal [symbol] helical domain of Nef, is necessary for maximal MHC-I degradation (Schaefer et al., 2008). The second site, a di-acidic motif in the C-terminal loop domain of Nef, is needed for efficient CD4 degradation (Piguet et al., 1999). To assess whether CMA affects beta-COP binding, s Nef-expressing or control T cells treated with solvent control or low dose CMA is utilized. Beta-COP-Nef complexes are immunoprecipitated from lysates using a control antibody or an antibody directed against beta-COP (M3A5) as previously described (Schaefer et al., 2008). The presence of co-precipitating Nef protein is detected by western blot analysis. In addition to wild type Nef, the Nef proteins previously generated (Schaefer et al., 2008) that are mutated at each of the two separate beta-COP binding site are utilized.

### Example 29

Determine whether an interaction between Nef and the V1H subunit of V-ATPase is needed for Nef-dependent MHC-1 downmodulation: Interestingly, Nef has been reported to interact with a component of the V1 complex of V-ATPase (V1H) (Geyer et al., 2002; Lu et al., 1998) and this interaction may facilitate the interaction between Nef and AP-2 to induce CD4 endocytosis (Geyer et al., 2002; Lu et al., 1998). The interaction between Nef and V1H has not yet been linked to Nef-dependent MHC-I downmodulation, where the interaction between Nef, MHC-I and AP-1 has been shown to be direct based on X-ray crystal structure (Jia et al., 2012) and cryo-electron microscopy (Shen et al., 2015). Nevertheless, it is possible that the reported interaction between V-ATPase and Nef is important for a subsequent step in the pathway needed for Nef-mediated disruption of MHC-I trafficking to the lysosome. To assess this possibility, the western blots generated in Example 7 are utilized with antibodies directed against the V1H subunit to determine whether it is a component of these complexes and if so, to determine whether CMA affects its ability to interact with Nef. In addition, shRNA directed against the V1H subunit is generated and determined whether silencing this subunit of V-ATPase alters Nef-dependent MHC-I trafficking. For the silencing studies, the same lentiviral vector-based shRNA is employed a silencing system previously used to silence AP-1 [symbol] subunit expression in T cells (Roeth et al., 2004) and then the amount of surface MHC-I in control or Nef-expressing T cells plus or minus V-ATPase subunit H silencing is measured. Silencing-dependent reversal of MHC-I downmodulation in Nef-expressing T cells would provide confirmatory evidence that V1H plays a role in Nef-dependent MHC-I downmodulation.

### Example 30

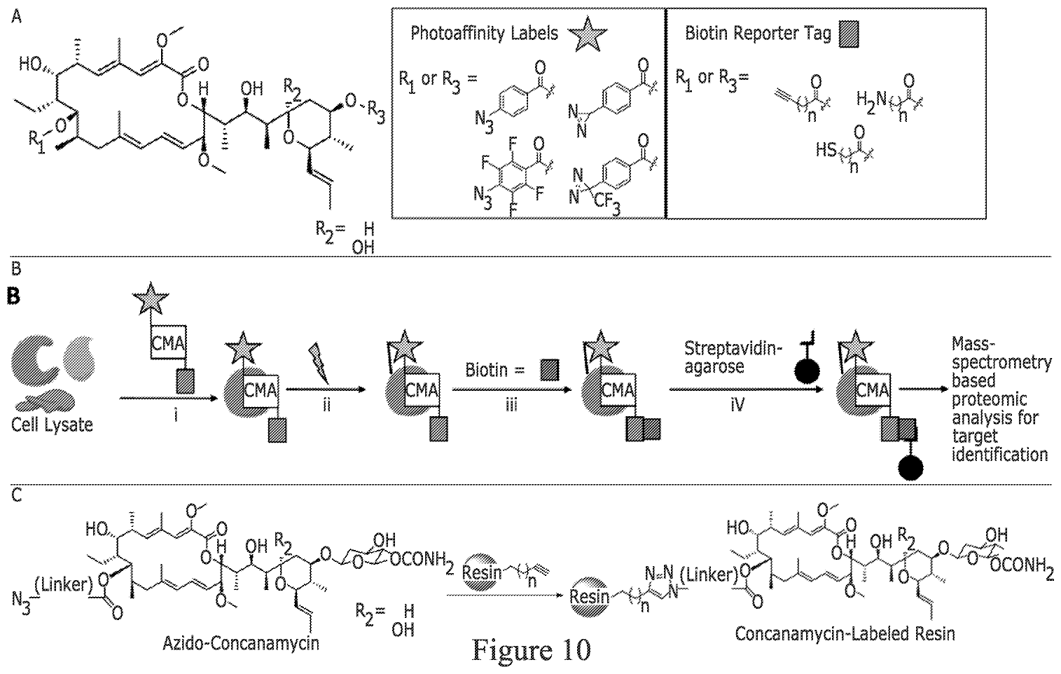

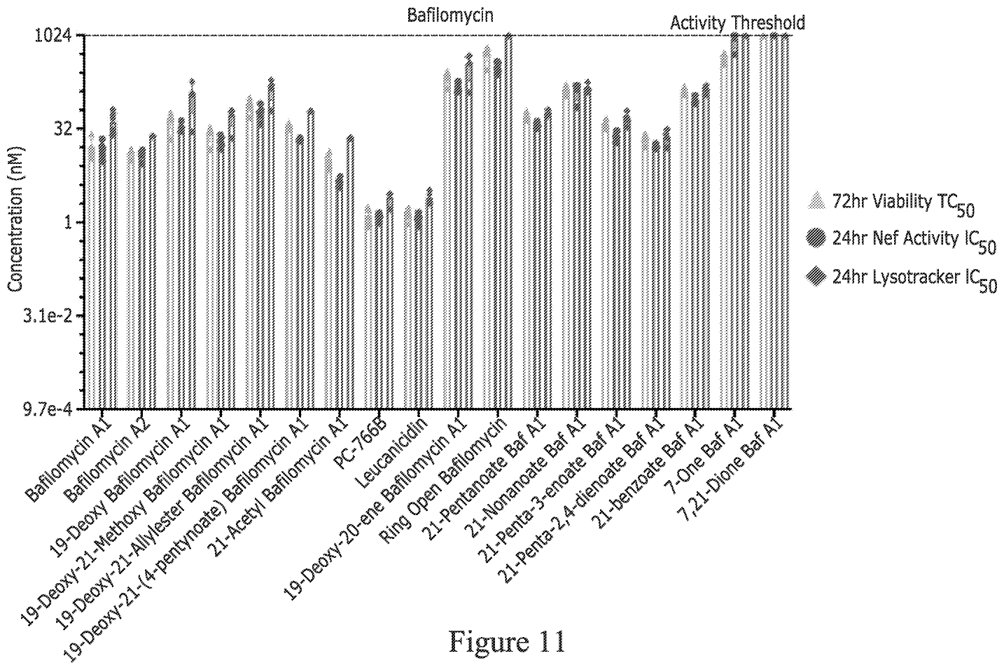

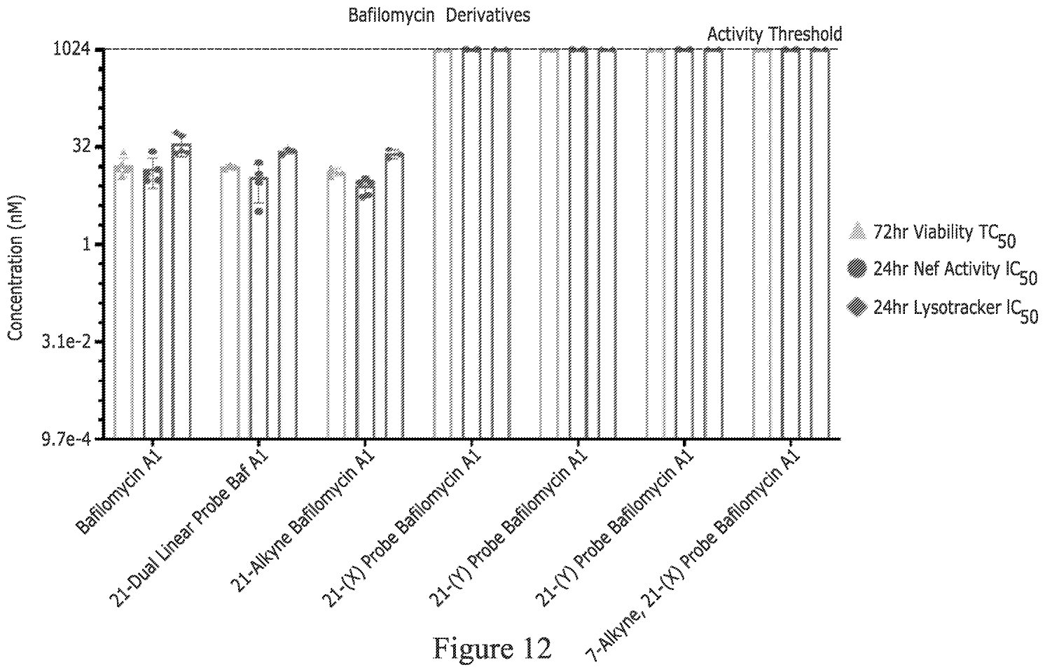

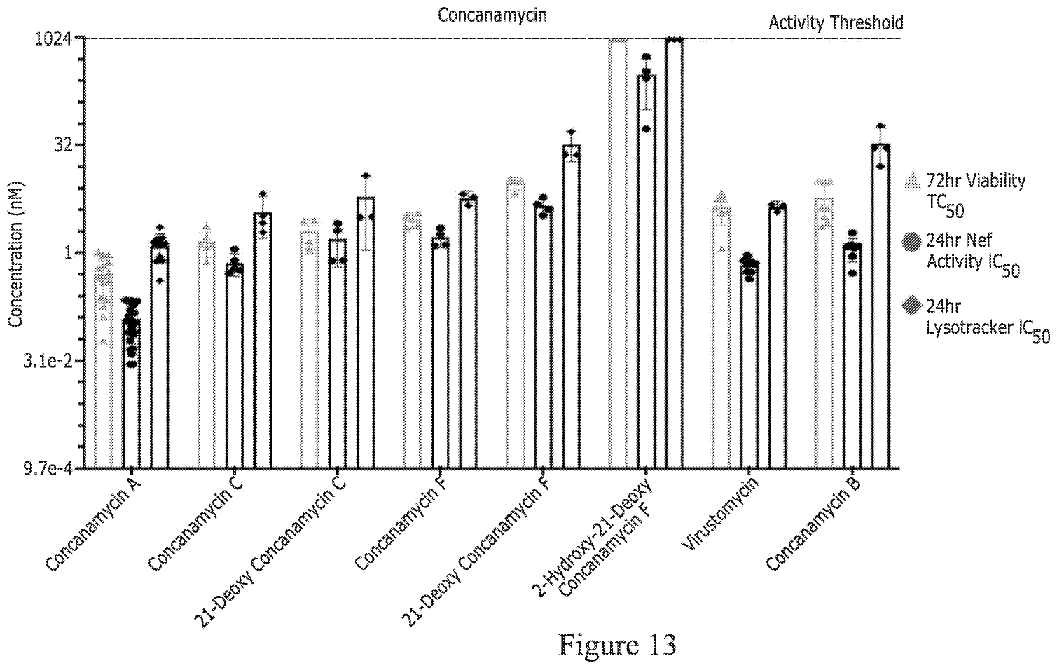

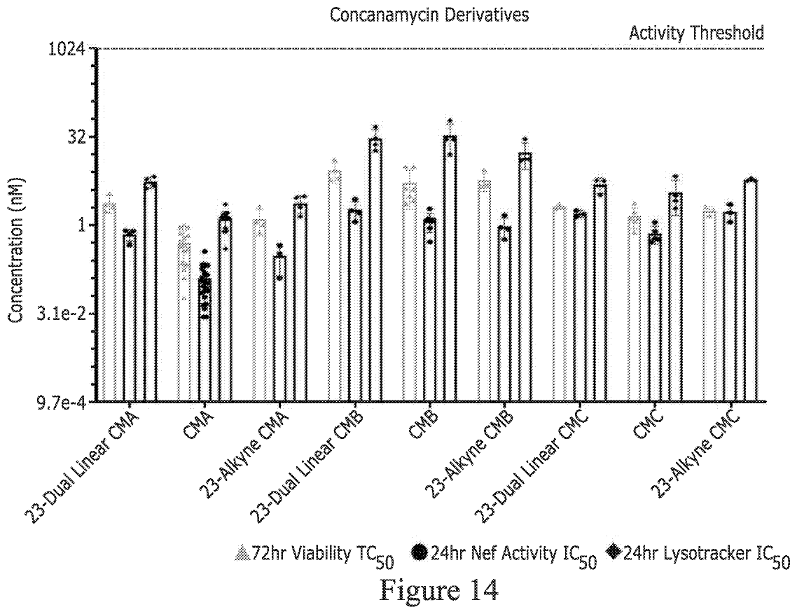

Identification of CMA target proteins in human PBMCs using affinity chromatography, affinity labeling, and proteomics approaches: In parallel with the biochemical studies described above, complementary unbiased approaches are used to identify all proteins that CMA can bind to in PBMCs. To accomplish this, lysates are treated from infected and uninfected PBMCs with doubly labeled CMA anologs, possessing both a photoaffinity tag and a secondary selective chemical handle (FIG. 10A). Treated cell lysates are exposed to UV-irradiation to covalently link CMA to target proteins. This is selectively coupled with activated biotin using the secondary chemical handle, yielding a protein-CMA-biotin conjugate (Smith and Collins, 2015). This conjugate is subjected to biotin-affinity chromatography using streptavidin-agarose to concentrate target proteins and reduce sample complexity (FIG. 10B). Eluted target proteins are identified by mass spectrometry based proteomics approaches (Rath et al., 2011). Analysis is performed using an Orbitrap instrument. The synthesis and use of photoaffinity labeled-CMAs has been reported (Dröse et al., 2001; Ingenhorst et al., 2001) for covalently linking CMA to V-ATPases, illustrating the utility of this approach. The advantages of this methodology lie in the tactical introduction of pliant functional groups that can be subsequently manipulated. Positions of labels on the core CMA scaffold can be altered, linker lengths modified, photoaffinity groups exchanged (phenyl azides, diazirines, etc.), and biotin linker arms substituted, all of which can ultimatetly be used to improve target affinity and probe reactivity. This methodology is used to identify novel CMA-protein interactions involved in disruption of Nef-mediated MHC-I downmodulation and further understanding of Nef-dependent MHC-I trafficking and guide further development of selective Nef-inhibitors.

These studies determine how Baf/CMA selectively block MHC-I downmodulation by targeting a particular step Nef utilizes to downmodulate MHC-I. The inhibitors may affect Nef protein localization or alter the ability of Nef to interact with MHC-I, AP-1, ARF-1 and/or [symbol] COP. Because V-ATPase subunits have been reported to interact with Nef, it is possible that these studies demonstrate a previously unknown role for this subunit in Nef-dependent MHC-I downmodulation as well as CMA-dependent disruption of this functional interaction. In parallel unbiased approaches identify all protein targets of CMA. The results of this analysis confirm that CMA binds to the Vₒ c subunit. In addition, new protein(s) expressed in primary T cells that binds to CMA at lower concentrations (higher binding affinity) than the Vₒ c subunit can be identified. These targets could potentially include components of the Nef-MHC-I-AP-1 complex. By determining the mechanism by which Baf/CMA affect Nef and by determining the protein CMA selectively targets at high affinity, rational strategies to modify Baf/CMA analogs can be identified to enhance efficacy and limit toxicity. Moreover, this work improves understanding of how Nef operates in HIV infected T cells.

To ensure recovery of all target molecules, CMA-coupled-resins are utilized to isolate targets via affinity chromatography. This approach has been shown to be an effective method for target identification of other small molecules (Azarkan et al., 2007). Baf C1-labeled cellulose has been previously prepared and used to identify V-ATPases as the main targets for the plecomacrolides (Rautiala et al., 1993). A CMA-coupled resin is used for affinity chromatography studies in a similar fashion. Commercially available coupling-resins specifically designed for small-molecule linked resin protocols are either directly linked with CMA, or to a semisynthetic derivative of CMA containing an installed chemical handle (alkyne, amine, carboxyl, sulfhydryl etc.). This is performed in parallel with the production of the doubly labeled-CMA derivatives, taking advantage of chemical handles installed for biotin linking (FIG. 10C). CMA affinity-chromatography is conducted on cell lysates from both infected and uninfected human PBMCs. Eluted target proteins are subsequently identified utilizing mass spectrometry-based proteomics approaches previously described.

### REFERENCES

Alberti, F. et al 2019. Nat Prod Rep.
Anzai, Y. et al. 2008. Chem Biol. 15:950-959.
Archin, N. M. et al. 2012. Nature. 487:482-485.
Azarkan, M. et al. 2007. Journal of chromatography. B, Analytical technologies in the biomedical and life sciences. 849:81-90.
Bindseil, K. U. et al. 1993. The Journal of Organic Chemistry. 58:5487-5492.
Carlson, J. C. et al. 2009. Journal of natural products. 72:2076-2079.
Carlson, J. C. et al. 2011. Nature chemistry. 3:628-633.
Chen, B. et al. 1996. J. Virol. 70:6044-6053.
Coleman, S. H. et al. 2005. Journal of virology. 79:2066-2078.
Collins, K. L. et al. 1998. Nature. 391:397-401.
Contreras, X. et al. 2009. The Journal of biological chemistry. 284:6782-6789.
Cruz, P. G. et al. 2011. Chemistry & biology. 18:1442-1452.
Deeg, M. et al. 1987. The Journal of antibiotics. 40:320-328.
Deeks, S. G. 2012. Nature. 487:439-440.
Drose, S. et al. 1997. J Exp Biol. 200:1-8.
Dröse, S. et al. 2001. Biochemistry. 40:2816-2825.
Effenberger, K. A. et al. 2017. Wiley Interdiscip Rev RNA. 8.
Elshahawi, S. I. et al. 2015. Chemical Society Reviews. 44:7591-7697.
Forgac, M. 2007. Nat Rev Mol Cell Biol. 8:917-929.
Fribley, A. M. et al. 2011. Journal of biomolecular screening. 16:825-835.
Fu, X. et al. 2003. Nature Biotechnology. 21:1467.
Geyer, M. et al. 2002. The Journal of biological chemistry. 277:28521-28529.
Gilbert, M. M. et al. 2017. Acs Central Sci. 3:1304-1310.
Greenberg, M. E. et al. 1997. Embo J. 16:6964-6976.
Hansen, D. A. et al. 2013. J Am Chem Soc. 135:11232-11238.
Haydock, S. F. et al. 2005. ATCC 27449. 151:3161-3169.
Heigele, A. et al. 2012. Journal of virology. 86:36-48.
Hettiarachchi, K. D. et al. 2006. Food Chem Toxicol. 44:1966-1977.
Huss, M. et al. 2002. The Journal of biological chemistry. 277:40544-40548.
Ingenhorst, G. et al. 2001. European Journal of Organic Chemistry. 2001:4525-4532.
Ishii, T. et al. 1995. The Journal of antibiotics. 48:12-20.
Janvier, K. et al. 2001. Journal of virology. 75:3971-3976.
Jia, X. et al. 2012. Nature structural & molecular biology. 19:701-706.
Jin, Y. J. et al. 2005. Journal of immunology. 175:3157-3164.
Jung, W. S. et al. 2008. Appl Environ Microb. 74:1972-1979.
Jung, W. S. et al. 2006. Appl Microbiol Biotechnol. 72:763-769.
Kasper, M. R. et al. 2003. Journal of virology. 77:3041-3049.
Kasper, M. R. et al. 2005. The Journal of biological chemistry. 280:12840-12848.
Lehrman, G. et al. 2005. Lancet. 366:549-555.
Leonard, J. A. et al. 2011. Journal of virology. 85:6867-6881.
Li, J. W. et al. 2009. Science. 325:161-165.
Li, S. et al. 2009. Proceedings of the National Academy of Sciences of the United States of America. 106:18463-18468.
Li, Z. et al. 2017. The Journal of biological chemistry. 292:7095-7104.
Lowell, A. N. et al. 2017. Journal of the American Chemical Society. 139:7913-7920.
Lu, X. et al. 1998. Immunity. 8:647-656.
Mike, L. A. et al. 2017. Chem Commun. 53:12778-12781.
Morpurgo, M. et al. 1999. Journal of Biochemical and Biophysical Methods. 38:17-28.
Mujib, S. et al. 2017. JCI Insight. 2.
Narayan, A. R. et al. 2015. Nat Chem. 7:653-660.
Negretti, S. et al. 2014. Journal of the American Chemical Society. 136:4901-4904.
Newman, D. J., et al. 2016. J Nat Prod. 79:629-661.
Omondi, F. H. et al. 2019. Journal of virology.
Omura, S. 2016. Angew Chem Int Ed Engl. 55:10190-10209.
Parham, P. et al. 1981. Human immunology. 3:277-299.
Park, S. R. et al. 2016. Nature communications. 7:10710.
Piguet, V. et al. 1999. Cell. 97:63-73.
Rath, C. M. et al. 2011. ACS Chem Biol. 6:1244-1256.
Rautiala, T. J. et al. 1993. Biochemical and biophysical research communications. 194:50-56.
Raveh, A., et al. 2013. PloS one. 8:e82318.
Richman, D. D. et al. 2009. Science. 323:1304-1307.
Robertson, A. W. et al. 2015. Journal of the American Chemical Society. 137:3271-3275.
Roeth, J. F. et al. 2004. J Cell Biol. 167:903-913.
Schaefer, M. R. et al. 2008. PLoS pathogens. 4:e1000131.
Shan, L. et al. 2012. Immunity. 36:491-501.
Shareef, A. R. et al. 2012. Chem Sci. 3:892-895.
Shen, Q. T. et al. 2015. Science. 350:aac5137.
Smith, E. etal. 2015. Future Med Chem. 7:159-183.
Stam, N. J. et al. 1986. Journal of immunology. 137:2299-2306.
Tay, J.-H., et al. 2017. R Journal of the American Chemical Society. 139:8570-8578.
Tong, X. S. et al. 2006. Journal of chromatography. B, Analytical technologies in the biomedical and life sciences. 833:165-173.
Tripathi, A. et al. 2014. Journal of the American Chemical Society. 136:1579-1586.
Trunzer, M. et al. 2009. Journal of medicinal chemistry. 52:329-335.
Williams, M. et al. 2002. Journal of virology. 76:12173-12184.
Wonderlich, E. R. et al. 2011. Journal of virology. 85:12216-12226.
Wonderlich, E. R. et al. 2008. The Journal of biological chemistry. 283:3011-3022.
Yuan, N. et al. 2015. Haematologica. 100:345-356.
Zhang, T. et al. 2013a. Biopharmaceutics & drug disposition. 34:348-359.
Zhang, W. et al. 2013b. Chembiochem. 14:301-306.
Zou, P. et al. 2012. Journal of pharmacy & pharmaceutical sciences: a publication of the Canadian Society for Pharmaceutical Sciences, Societe canadienne des sciences pharmaceutiques. 15:265-280.

What is claimed is:

1. A compound having a structure of Formula (I), or a pharmaceutically acceptable salt thereof: 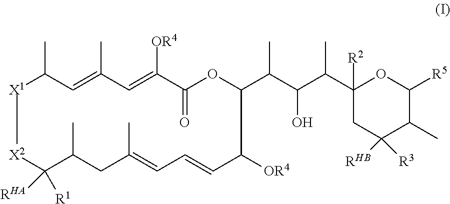
wherein 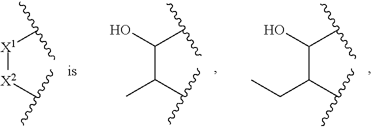
or represents a direct bond;
R^{HA} and R^{HB} are H;
R¹ is OH or OC(O)R⁶,
or R^{HA} and R¹ together with the carbon to which they are attached form an oxo (═O) group;
R² is H, OH, or OCH₃;
R³ is O—C₁₋₆alkyl, O—C₁₋₆alkenyl, O—C(O)R⁷, or 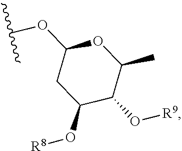
or R^{HB} and R³ together with the carbon to which they are attached form an oxo (═O) group;
each R⁴ is independently H or C₁₋₆alkyl;
R⁵ is C₁₋₆alkyl or C₂₋₆alkenyl;
R⁶ is C₂₋₆ alkynyl or C₆₋₁₀ aryl, each optionally substituted with 1 to 3 R¹⁰;
R⁷ is C₁₋₈ alkyl, C₂₋₆ alkenyl, C₂₋₆ alkynyl, or C₆₋₁₀ aryl, each optionally substituted with 1 to 3 R¹⁰;
R⁸ is H or C(O)R⁶;
R⁹ is H or C(O)NH₂, and
R¹⁰ is C₁₋₆haloalkyl or (C₂alkylene-O)₂—C₂₋₄alkynyl, wherein the haloalkyl and alkylene are optionally substituted with a fused 3-membered heterocyloakyl ring comprising two nitrogen atoms;
with the provisos that (i) when R² is OH or OCH₃ then R⁸ is not H, and R^{HB} and R³ together with the carbon to which they are attached do not form an oxo (═O) group, and (ii) when R¹ is OH, then R⁶ is not C₆₋₁₀ aryl.

2. The compound or salt of claim 1, having a structure of Formula (Ia), (Ib), or (Ic): 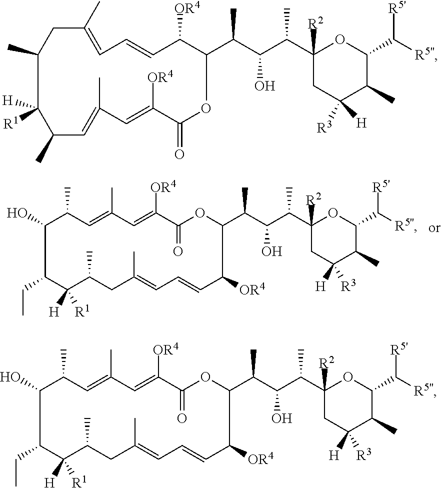
wherein (i) R^{5′} is C₂₋₅alkenyl and R^{5″} is H, or (ii) both R^{5′} and R^{5″} are C₁₋₂alkyl.

3. The compound or salt claim 1 or 2, wherein R¹ is OH.

4. The compound or salt claim 1 or 2, wherein R¹ is OC(O)R⁶.

5. The compound or salt of claim 4, wherein R¹ is

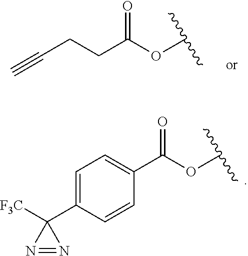

6. The compound or salt of claim 1, wherein R^{HA} and R¹ together with the carbon to which they are attached form an oxo (═O) group.

7. The compound or salt of any one of claims 1 to 6, wherein R² is H.

8. The compound or salt of any one of claims 1 to 6, wherein R² is OH.

9. The compound or salt of any one of claims 1 to 8, wherein R³ is O—C₁₋₆alkyl, or O—C₁₋₆alkenyl.

10. The compound or salt of claim 9, wherein R³ is OCH₃, or

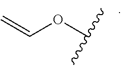 [SMILES: *OC=C]

11. The compound or salt of any one of claims 1 to 8, wherein R³ is O—C(O)R⁷.

12. The compound or salt of claim 11, wherein R⁷ is C₁₋₈ alkyl, C₂₋₆ alkenyl, or C₂₋₆ alkynyl, each optionally substituted with 1 to 3 R¹⁰.

13. The compound or salt of claim 12, wherein R³ is

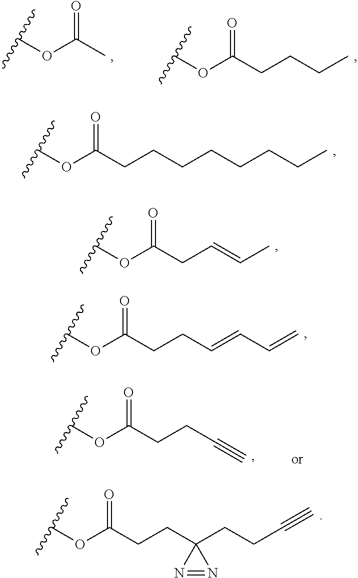

14. The compound or salt of claim 11, wherein R⁷ is C₆₋₁₀ aryl optionally substituted with 1 to 3 R¹⁰.

15. The compound or salt of claim 14, wherein R³ is

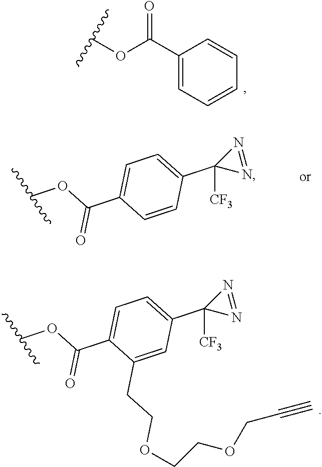 [SMILES: *OC(=O)c1ccccc1 ; *OC(=O)c1ccc(C2(C(F)(F)F)N=N2)cc1 ; *OC(=O)c1ccc(C2(C(F)(F)F)N=N2)cc1CCOCCOCC#C]

16. The compound or salt of any one of claims 1 to 8, wherein R³ is

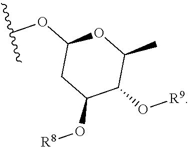

17. The compound or salt of claim 16, wherein R⁸ is H.

18. The compound or salt of claim 16, wherein R⁸ is C(O)R⁶.

19. The compound or salt of claim 18, wherein R⁸ is

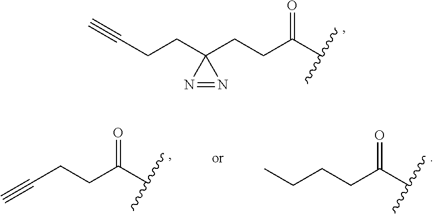

20. The compound or salt of any one of claims 16 to 19, wherein R⁹ is H.

21. The compound or salt of any one of claims 16 to 19, wherein R⁹ is C(O)NH₂.

22. The compound or salt of claim 1, wherein R^{HB} and R³ together with the carbon to which they are attached form an oxo (═O) group.

23. The compound or salt of any one of claims 1 to 22, wherein at least one R⁴ is C₁₋₆alkyl.

24. The compound or salt of any one of claims 1 to 22, wherein each R⁴ is C₁₋₆alkyl.

25. The compound or salt for use of claim 23 or 24, wherein at least one R⁴ is methyl.

26. The compound or salt of claim 25, wherein each R⁴ is methyl.

27. The compound or salt of any one of claims 1 to 26, wherein R⁵ is C₁₋₆alkyl.

28. The compound or salt of any one of claims 1 to 26, wherein R⁵ is C₂₋₆alkenyl.

29. The compound or salt of any one of claims 2 to 28, wherein R^{5′} is C₂₋₅alkenyl and R^{5″} is H.

30. The compound or salt of claim 29, wherein R⁵ is

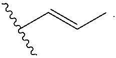 [SMILES: */C=C/C]

31. The compound or salt of any one of claims 2 to 28, wherein both R^{5′} and R^{5″} are C₁₋₂alkyl.

32. The compound or salt of claim 31, wherein R⁵ is

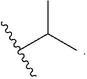 [SMILES: *C(C)C]

33. A compound, or pharmaceutically acceptable salt thereof, having a structure as shown in Table A.

34. The compound or salt any one of claims 1 to 33 in the form of a salt.

35. A pharmaceutical composition comprising the compound of salt of any one of claims 1 to 33 and a pharmaceutically acceptable excipient.

36. A method of modulating human immunodeficiency virus (HIV) Nef and its allotypes comprising administering to a patient in need thereof a pharmaceutically-effective amount of the compound or salt of any one of claims 1 to 34 or the pharmaceutical composition of claim 35.

37. The method of claim 36, wherein modulating HIV Nef and its allotypes comprises inhibiting HIV Nef and its allotypes.

38. A method of treating human immunodeficiency virus (HIV) infection comprising administering to a patient in need thereof a pharmaceutically-effective amount of the compound or salt of any one of claims 1 to 34 or the pharmaceutical composition of claim 35.

39. The method of claim 38, wherein the HIV infection is HIV-1 infection.

40. The method of claim 39, wherein the HIV-1 infection is infection with HIV subtype A, B, C, D, E, F, G, H, I, J, K, L, or a recombination thereof.

41. The method of any one of claims 38 to 40, wherein treating HIV infection comprises reducing an HIV reservoir in a host.

42. The method of any one of claims 38 to 41, wherein treating HIV infection comprises eliminating an HIV reservoir in a host.

43. The compound or salt of any one of claims 1 to 34 or the composition of claim 35 for use as a medicament for modulating human immunodeficiency virus (HIV) Nef and its allotypes in a patient.

44. The compound or composition of claim 43, wherein modulating HIV Nef and its allotypes comprises inhibiting HIV Nef and its allotypes.

45. The compound or salt of any one of claims 1 to 34 or the composition of claim 35 for use as a medicament for treating human immunodeficiency virus (HIV) infection in a patient.

46. The compound or composition of claim 45, wherein the HIV infection is HIV-1 infection.

47. The compound or composition of claim 46, wherein the HIV-1 infection is infection with HIV subtype A, B, C, D, E, F, G, H, I, J, K, L, or a recombination thereof.

48. The compound or composition of any one of claims 45 to 47, wherein treating HIV infection comprises reducing an HIV reservoir in a host.

49. The compound or composition of any one of claims 45 to 48, wherein treating HIV infection comprises eliminating an HIV reservoir in a host.
